SEQUENCE LISTING (35 sequences)

SEQ ID NO: 1 - protein, 299 aa, unidentified
MGLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVETVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKADQESSEPHAAIGAAANKSGGSLIS

SEQ ID NO: 2 - protein, 492 aa, unidentified
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFAYLEQHYMPGLRSQLVTHRMFTPFDFRDQLNAYHGSAFSVEPVLTQSAWFRPHNRDKTITNLYLVGAGTHPGAGIPGVIGSAKATAGLMLEDLI

SEQ ID NO: 3 - protein, 382 aa, unidentified
MQPHYDLILVGAGLANGLIALRLQQQQPDMRILLIDAAPQAGGNHTWSFHHDDLTESQHRWIAPLVVHHWPDYQVRFPTRRRKLNSGYFCITSQRFAEVLQRQFGPHLWMDTAVAEVNAESVRLKKGQVIGARAVIDGRGYAANSALSVGFQAFIGQEWRLSHPHGLSSPIIMDATVDQQNGYRFVYSLPLSPTRLLIEDTHYIDNATLDPECARQNICDYAAQQGWQLQTLLREEQGALPITLSGNADAFWQQRPLACSGLRAGLFHPTTGYSLPLAVAVADRLSALDVFTSASIHHAITHFARERWQQQGFFRMLNRMLFLAGPADSRWRVMQRFYGLPEDLIARFYAGKLTLTDRLRILSGKPPVPVLAALQAIMTTHR

SEQ ID NO: 4 - protein, 302 aa, unidentified
MTVCAKKHVHLTRDAAEQLLADIDRRLDQLLPVEGERDVVGAAMREGALAPGKRIRPMLLLLTARDLGCAVSHDGLLDLACAVEMVHAASLILDDMPCMDDAKLRRGRPTIHSHYGEHVAILAAVALLSKAFGVIADADGLTPLAKNRAVSELSNAIGMQGLVQGQFKDLSEGDKPRSAEAILMTNHFKTSTLFCASMQMASIVANASSEARDCLHRFSLDLGQAFQLLDDLTDGMTDTGKDSNQDAGKSTLVNLLGPRAVEERLRQHLQLASEHLSAACQHGHATQHFIQAWFDKKLAAVS

SEQ ID NO: 5 - protein, 309 aa, unidentified
MNNPSLLNHAVETMAVGSKSFATASKLFDAKTRRSVLMLYAWCRHCDDVIDDQTLGFQARQPALQTPEQRLMQLEMKTRQAYAGSQMHEPAFAAFQEVAMAHDIAPAYAFDHLEGFAMDVREAQYSQLDDTLRYCYHVAGVVGLMMAQIMGVRDNATLDRACDLGLAFQLTNIARDIVDDAHAGRCYLPASWLEHEGLNKENYAAPENRQALSRIARRLVQEAEPYYLSATAGLAGLPLRSAWAIATAKQVYRKIGVKVEQAGQQAWDQRQSTTTPEKLTLLLAASGQALTSRMRAHPPRPAHLWQRPL

SEQ ID NO: 6 - protein, 393 aa, unidentified
MHRITILGATGSIGESTLDVVRRHADRYVVHALTAHRQVRKLADQCVEFRPARAVVGTAEAALELETLLRDAGVKTEVSHGEAALESVAADAQTDSVMAAIVGAAGLRPTLAAARAGKRVLLANKEALVMSGRIFMDAVREHGATLLPIDSEHNAIFQCLPADDPRYGRGVARVLLTASGGPFRTRDPATLHDISPDQACAHPNWVMGRKISVDSATMMNKGLEVIEAHWLFGAPAERIEVLIHPQSIVHSMVAYTDGSVLAQLGNPDMRTPIAYGLAYPERIDAGVTPLDLTVAGGLHFEKPDLVRFPCLGLAFDALRAGGVAPAALNAANEVAVEAFLGGTVRFTDIAGIVRQVLEATPQGPADTLEAVLSADALAREAAREGVAALAAKR

SEQ ID NO: 7 - protein, 308 aa, unidentified
MAQPRGFCAGVDRAIEIVERALERFGAPIYVRHEIVHNAYVVAGLRRKGAVFVRELDEVPAGATVIFSAHGVSREVRADAAARGLHVFDATCPLVTKVHVEVSKMRAEGCEIVMIGHRGHPEVEGTMGQASSGMLLVESVADVATLQVTDPSRLAYVTQTTLSVDETREIVAALKARFPQIREPKKQDICYATQNRQDAVKFMAPQVEVVIVVGSPNSSNSNRLRELAERLGVPAYMVDAPEQVRPEWIAGKRRIGLTAGASAPEALAQSIVERLRELGASQVRPLDGIEENMAFPLPRGLLPASAAA

SEQ ID NO: 8 - protein, 294 aa, unidentified
MSDFAQWMQAQGARTEAALQAALPAAETVPHTLHEAMRYAALSGGKRVRPLLVHAAGEVSGAAPAACDAAACAVEMIHAYSLVHDDMPCMDDDDLRRGRPTVHKAYDEATALLVGDALQTQAFIVLAGAGAIAPAARLQLVAELALASGSTGMAGGQAIDLQNVGRAMTREALEAMHRMKTGALLRASVRMGALCGEIDAEGLAALDRYAAAVGLAFQVVDDILDVTADTATLGKTAGKDAAHDKPTYVSLMGLDPARALAGTLRADAHEALAGFGERADRLRDLADLIVLRTH

SEQ ID NO: 9 - DNA, 6577 bp, Pantoea ananatis
  source 1..6577: mol_type = unassigned DNA
gtgcaacgttatggattgatggcgcttttgctcgtttcctgctgggccagcgcgcataacatcgtcatcgggcagccccttccgtcggtttttattgcggataaaggtgaaatgcggctggatggcggcaaggttaactatcaaaaatggaacagcctgtctcttccgggtcggacacgtttagttattcatgttgcaggacgattgtcggccaaagagcagtccgccccgcttattgcggccctgcagcgcgccaacctgccacaagaccggttccagaccacaaccatcgtgaatacagatgatgctttgcctggcagcagtctgtttgtgattaacagtatccgctccagtaaaaaagcctcaccatggcaacaatttattatcgacagtagcggcgtggcacaacatcgctggcagcttaagccagaaggtgccgctgtcatcgtgctggaccctgatggtcaggtaaagtttgcgaaagacacggcgctcagtgcggatgatgtttctcaggtcattgcaacattgcgtgcgctggcaggctgatcctggcaacccggtaaaggtaccgcacggtctgccaatccgacggaggtttatgaattttccaccttttccacaagctcaactagtattaacgatgtggatttagcaaaaaaaacctgtaaccctaaatgtaaaataacgggtaagcctgccaaccatgttatggcagattaagcgtctttttgaagggcaccgcatctttcgcgttgccgtaaatgtatccgtttataaggacagcccgaatgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaaggatgctgcatgagccatttcgcggcgatcgcaccgcctttttacagccatgttcgcgcattacagaatctcgctcaggaactggtcgcgcgcggtcatcgggtgacctttattcagcaatacgatattaaacacttgatcgatagcgaaaccattggatttcattccgtcgggacagacagccatccccccggcgcgttaacgcgcgtgctacacctggcggctcatcctctggggccgtcaatgctgaagctcatcaatgaaatggcgcgcaccaccgatatgctgtgccgcgaactcccccaggcatttaacgatctggccgtcgatggcgtcattgttgatcaaatggaaccggcaggcgcgctcgttgctgaagcactgggactgccgtttatctctgtcgcctgcgcgctgcctctcaatcgtgaaccggatatgcccctggcggttatgcctttcgaatacgggaccagcgacgcggctcgcgaacgttatgccgccagtgaaaaaatttatgactggctaatgcgtcgtcatgaccgtgtcattgccgaacacagccacagaatgggcttagcccccccggcaaaagcttcaccagtgtttttcgccactggcgcaaatcagccagcttgttcctgaactggattttccccgcaaagcgttaccggcttgttttcatgccgtcgggcctctgcgcgaaacgcacgcaccgtcaacgtcttcatcccgttattttacatcctcagaaaaaccccggattttcgcctcgctgggcacgcttcagggacaccgttatgggctgtttaaaacgatagtgaaagcctgtgaagaaattgacggtcagctcctgttagcccactgtggtcgtcttacggactctcagtgtgaagagctggcgcgaagccgtcatacacaggtggtggattttgccgatcagtcagccgcgctgtctcaggcgcagctggcgatcacccacggcggcatgaatacggtactggacgcgattaattaccggacgccccttttagcgcttccgctggcctttgatcagcccggcgtcgcgtcacgcatcgtttatcacggcatcggcaagcgtgcttcccgctttaccaccagccatgctttggctcgtcagatgcgttcattgctgaccaacgtcgactttcagcagcgcatggcgaaaatccagacagcccttcgtttggcagggggcaccatggccgctgccgatatcattgagcaggttatgtgcaccggtcagcctgtcttaagtgggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaaagagcgactacatgaaaccaactacggtaattggtgcaggcttcgntggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctaggatcagcggcagtcaacgaccacgcccgaaaaattaacgctgctgctggccgcctctggtcaggcccttactcccggatgcgggctcatcctccccgccctgcgcatctctggcagcgcccgctctagcgccatgtctttcccggagcgtc

SEQ ID NO: 10 - protein, 304 aa, unidentified
MGGLMLIDWCALALVVFIGLPHGALDAAISFSMISSAKRIARLAGILLIYLLLATAFFLIWYQLPAFSLLIFLLISIIHFGMADFNASPSKLKWPHIIAHGGVVTVWLPLIQKNEVTKLFSILTNGPTPILWDILLIFFLCWSIGVCLHTYETLRSKHYNIAFELIGLIFLAWYAPPLVTFATYFCFIHSRRHFSFVWKQLQHMSSKKMMIGSAIILSCTSWLIGGGIYFFLNSKMIASEAALQTVFIGLAALTVPHMILIDFIFRPHSSRIKIKNKGELEGKPIPNPLLGLDSTRTGHHHHHH

SEQ ID NO: 11 - protein, 393 aa, unidentified; identical to SEQ ID NO: 6

SEQ ID NO: 12 - DNA, 285 bp, unidentified
  source 1..285: mol_type = unassigned DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccat

SEQ ID NO: 13 - protein, 299 aa, unidentified; identical to SEQ ID NO: 1

SEQ ID NO: 14 - DNA, 18455 bp, synthetic construct
  source 1..18455: mol_type = other DNA
acatggtactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagctaaggaggagaccccatgggagagctcatgcatcgcattaccatcctgggcgccaccggctccattggcgaaagcacgctcgacgtcgtcaggcgccatgccgaccgctatgtggtgcatgcgctgaccgcccaccggcaggtgcgcaagctggctgaccagtgcgttgagttccgcccggcccgcgccgtggtggggaccgccgaggcagcgctcgaactggaaaccctgctgcgcgatgccggcgtgaagaccgaggtcagccacggcgaagccgcgctggaatccgtggccgccgatgcgcagaccgattcggtgatggccgccatcgtcggcgccgccggcctgcgcccgacgctggcggcagcacgtgccggcaagcgcgtgctgctggccaacaaggaagcgctggtgatgtccgggcgcatcttcatggatgcggtgcgcgagcacggcgccaccttgctgccgatcgacagcgagcacaacgccatcttccagtgccttccggcagatgatccgcgttatggtcgtggtgttgcaagggttctgttgaccgcatcaggaggtccgttccgtacgcgtgacccggcaacgctgcacgatatctcccccgaccaggcatgcgcacatcccaactgggttatgggtcgcaagatctcggtcgattccgccaccatgatgaacaagggcctcgaagtcatcgaggcgcactggctgttcggcgcccccgccgagcgcatcgaggtactgatccatccgcagagcatcgtgcattcgatggtggcctacaccgacggctcggtgctggcgcaactgggcaacccggacatgcgcacgccgatcgcctatggcctggcgtatccggagcggatcgatgcgggggtcacgccgctggacctgaccgtggctgggggcttgcatttcgagaagccggacctggtgcgcttcccgtgcctgggcctggccttcgatgcactgcgcgccggcggtgtggcgccggcggccctcaatgcagccaacgaggtggcggtcgaggcattcctgggcggtacagtgcgcttcacggacattgccggtatcgtccggcaggtgcttgaggcgacaccccaggggcctgccgatacgctcgaagcggtgctgtcagccgacgcgctggcgcgcgaggcggcacgcgagggcgtggccgccctggctgcaaagcgctgaagcttaaggaggagaccccatgggcggcctgatgctgatcgactggtgcgccctggccctggtggtctttattggtctcccgcacggggcactggacgcggccatctcgttctcgatgatctcgtcggccaagcgcatcgcccgcctggccggcatcctgctgatctacctgctgctggccaccgccttcttcctgatctggtaccagctgccggccttctcgctgctgatcttcctgctgatctcgatcatccacttcggcatggccgacttcaacgcctcgccgtcgaagctgaagtggccgcacatcatcgcccacggcggcgtggtgaccgtgtggctgccgctgatccagaagaacgaagtgaccaagctgttctcgatcctgaccaacggcccgaccccgatcctgtgggacatcctgctgatcttcttcctgtgctggtcgatcggcgtgtgcctgcacacctacgaaaccctgcgctcgaagcactacaacatcgccttcgaactgatcggcctgatcttcctggcctggtacgccccgccgctggtgaccttcgccacctacttctgcttcatccactcgcgccgccacttctcgttcgtgtggaagcagctgcagcacatgtcgtcgaagaagatgatgatcggctcggccatcatcctgtcgtgcacctcgtggctgatcggcggcggcatctacttcttcctgaactcgaagatgatcgcctcggaagccgccctgcagaccgtgttcatcggcctggccgccctgaccgtgccgcacatgatcctgatcgacttcatcttccgcccgcactcgtcgcgcatcaagatcaagaacaagggcgaactggaaggcaagccgatcccgaacccgctgctgggcctggactcgacccgcaccggccatcatcaccatcaccattaagagctcgatctcaaacaggattgggcataaaacagttgaaagtgaataaagttgctaagctggtaactcagatcctaaaaacagggataagactgtgcaacgttatggattgatggcgcttttgctcgtttcctgctgggccagcgcgcataacatcgtcatcgggcagccccttccgtcggtttttattgcggataaaggtgaaatgcggctggatggcggcaaggttaactatcaaaaatggaacagcctgtctcttccgggtcggacacgtttagttattcatgttgcaggacgattgtcggccaaagagcagtccgccccgcttattgcggccctgcagcgcgccaacctgccacaagaccggttccagaccacaaccatcgtgaatacagatgatgctttgcctggcagcagtctgtttgtgattaacagtatccgctccagtaaaaaagcctcaccatggcaacaatttattatcgacagtagcggcgtggcacaacatcgctggcagcttaagccagaaggtgccgctgtcatcgtgctggaccctgatggtcaggtaaagtttgcgaaagacacggcgctcagtgcggatgatgtttctcaggtcattgcaacattgcgtgcgctggcaggctgatcctggcaacccggtaaaggtaccgcacggtctgccaatccgacggaggtttatgaattttccaccttttccacaagctcaactagtattaacgatgtggatttagcaaaaaaaacctgtaaccctaaatgtaaaataacgggtaagcctgccaaccatgttatggcagattaagcgtctttttgaagggcaccgcatctttcgcgttgccgtaaatgtatccgtttataaggacagcccgaatgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaaggatgctgcatgagccatttcgcggcgatcgcaccgcctttttacagccatgttcgcgcattacagaatctcgctcaggaactggtcgcgcgcggtcatcgggtgacctttattcagcaatacgatattaaacacttgatcgatagcgaaaccattggatttcattccgtcgggacagacagccatccccccggcgcgttaacgcgcgtgctacacctggcggctcatcctctggggccgtcaatgctgaagctcatcaatgaaatggcgcgcaccaccgatatgctgtgccgcgaactcccccaggcatttaacgatctggccgtcgatggcgtcattgttgatcaaatggaaccggcaggcgcgctcgttgctgaagcactgggactgccgtttatctctgtcgcctgcgcgctgcctctcaatcgtgaaccggatatgcccctggcggttatgcctttcgaatacgggaccagcgacgcggctcgcgaacgttatgccgccagtgaaaaaatttatgactggctaatgcgtcgtcatgaccgtgtcattgccgaacacagccacagaatgggcttagcccccccggcaaaagcttcaccagtgtttttcgccactggcgcaaatcagccagcttgttcctgaactggattttccccgcaaagcgttaccggcttgttttcatgccgtcgggcctctgcgcgaaacgcacgcaccgtcaacgtcttcatcccgttattttacatcctcagaaaaaccccggattttcgcctcgctgggcacgcttcagggacaccgttatgggctgtttaaaacgatagtgaaagcctgtgaagaaattgacggtcagctcctgttagcccactgtggtcgtcttacggactctcagtgtgaagagctggcgcgaagccgtcatacacaggtggtggattttgccgatcagtcagccgcgctgtctcaggcgcagctggcgatcacccacggcggcatgaatacggtactggacgcgattaattaccggacgccccttttagcgcttccgctggcctttgatcagcccggcgtcgcgtcacgcatcgtttatcacggcatcggcaagcgtgcttcccgctttaccaccagccatgctttggctcgtcagatgcgttcattgctgaccaacgtcgactttcagcagcgcatggcgaaaatccagacagcccttcgtttggcagggggcaccatggccgctgccgatatcattgagcaggttatgtgcaccggtcagcctgtcttaagtgggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaaagagcgactacatgaaaccaactacggtaattggtgcaggcttcgntggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctaggatcagcggcagtcaacgaccacgcccgaaaaattaacgctgctgctggccgcctctggtcaggcccttactcccggatgcgggctcatcctccccgccctgcgcatctctggcagcgcccgctctagcgccatgtctttcccggagcgtcagatctgcttggagccacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatctcgaattcaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagctaaggaggagaccccatgggtttgatgaccgtattttcttctgcacctgaacttgcccttctcggatcaacctttgcccaggtcgatccttcaaacttatcggtctcagattcgctgacctatggtcagttcaatctggtttacaacgctttctcgtttgccatcgcggcaatgttcgcatctgccctcttcttcttcagcgctcaggcactcgtcggtcaacgataccggttggccttgcttgtttcagcaattgttgtgagtatcgctgggtaccactactttcggatcttcaatagttgggatgctgcctacgttctggagaatggcgtgtattccctgactagcgaaaaattcaacgacgcctaccgctatgtggattggctgttgaccgtgcctctgttgctggtggagacagtggcagtgctgacgttgcctgcaaaggaggcaagacccttgctgatcaaactgacggtggcttcagttctgatgattgccacgggctaccccggcgagatttctgacgacattacgactcgcatcatctggggtacggtcagcacgattcccttcgcctacatcctctatgtgttgtgggtcgaactgtccaggtcccttgtccgccagcccgctgctgtacaaaccctggtccgcaacatgcggtggctgctgttgctctcctggggtgtttacccgatcgcataccttctacccatgcttggagtatccggtacgtccgcggctgtcggcgttcaggttggctatacgatcgcagacgtgctggcgaagcctgtatttggtcttctagtcttcgcgattgcactcgtgaaaacaaaagcagatcaagaaagcagtgaaccacatgccgcaataggtgctgctgcaaataaatcgggaggcagtcttatctcctagcggccgcagatctgcttggagccacccgcagttcgaaaaataataagcttgacctgtgaagtgaaaaatggcgcacattgtgcgacattttttttgtctgccgtttaccgctactgcgtcacagatctgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 15 - protein, 333 aa, synthetic construct [sequence omitted]

SEQ ID NO: 16 - protein, 697 aa, unidentified
MKFKLNLITLALLANTGLAVAADGYGLANANTEKVKLSAWSCKGCVVETGTSGTVGVGVGYNSEEDIRSANAFGTSNEVAGKFDADLNFKGEKGYRASVDAYQLGMDGGRLDVNAGKQGQYNVNVNYRQIATYDSNSALSPYAGIGGNNLTLPDNWITAGSSNQMPLLMDSLNALELSLKRERTGLGFEYQGESLWSTYVNYMREEKTGLKQASGSFFNQSMMLAEPVDYTTDTIEAGVKLKGDRWFTALSYNGSIFKNEYNQLDFENAFNPTFGAQTQGTMALDPDNQSHTVSLMGQYNDGSNALSGRILTGQMSQDQALVTDNYRYANQLNTDAVDAKVDLLGMNLKVVSKVSNDLRLTGSYDYYDRDNNTQVEEWTQISINNVNGKVAYNTPYDNRTQRFKVAADYRITRDIKLDGGYDFKRDQRDYQDRETTDENTVWARLRVNSFDTWDMWVKGSYGNRDGSQYQASEWTSSETNSLLRKYNLADRDRTQVEARITHSPLESLTIDVGARYALDDYTDTVIGLTESKDTSYDANISYMITADLLATAFYNYQTIESEQAGSSNYSTPTWTGFIEDQVDVVGAGISYNNLLENKLRLGLDYTYSNSDSNTQVRQGITGDYGDYFAKVHNINLYAQYQATEKLALRFDYKIENYKDNDAANDIAVDGIWNVVGFGSNSHDYTAQMLMLSMSYKL

SEQ ID NO: 17 - protein, 671 aa, unidentified
MMNAQKSKIALLLAASAVTMALTGCGGSDGNNGNDGSDGGEPAGSIQTLNLDITKVSYENGAPMVTVFATNEADMPVIGLANLEIKKALQLIPEGATGPGNSANWQGLGSSKSYVDNKNGSYTFKFDAFDSNKVFNAQLTQRFNVVSAAGKLADGTTVPVAEMVEDFDGQGNAPQYTKNIVSHEVCASCHVEGEKIYHQATEVETCISCHTQEFADGRGKPHVAFSHLIHNVHNANKAWGKDNKIPTVAQNIVQDNCQVCHVESDMLTEAKNWSRIPTMEVCSSCHVDIDFAAGKGHSQQLDNSNCIACHNSDWTAELHTAKTTATKNLINQYGIETTSTINTETKAATISVQVVDANGTAVDLKTILPKVQRLEIITNVGPNNATLGYSGKDSIFAIKNGALDPKATINDAGKLVYTTTKDLKLGQNGADSDTAFSFVGWSMCSSEGKFVDCADPAFDGVDVTKYTGMKADLAFATLSGKAPSTRHVDSVNMTACANCHTAEFEIHKGKQHAGFVMTEQLSHTQDANGKAIVGLDACVTCHTPDGTYSFANRGALELKLHKKHVEDAYGLIGGNCASCHSDFNLESFKKKGALNTAAAADKTGLYSTPITATCTTCHTVGSQYMVHTKETLESFGAVVDGTKDDATSAAQSETCFYCHTPTVADHTKVKM

SEQ ID NO: 18 - protein, 223 aa, unidentified
MTDAIAQLFRNNREWVDRVNAEDPTFFMRLANQQAPEYLWIGCSDSRVPANQILGLAPGEVFVHRNIANVIAHSDLNALAVIQFAVEVLKVRHITVVGHYGCGGVKVALKRERIGLADNWLRHVRDVADKHEAYLGTLLREDDAHTRLCELNVIEQVNNVCQTTVLQDAWSRGQAVTVHGWVYGVSDGLLRDLGMAASSNDELREQLAAAYRQYGDPPQASIR

SEQ ID NO: 19 - DNA, 32 bp, synthetic construct
  source 1..32: mol_type = other DNA
gatccatgggaagggatctgattttagtcggc

SEQ ID NO: 20 - DNA, 31 bp, synthetic construct
  source 1..31: mol_type = other DNA
gataagcttgctcattgcagatcctcaatca

SEQ ID NO: 21 - DNA, 32 bp, synthetic construct
  source 1..32: mol_type = other DNA
gctctagagctcgatctcaaacaggattgggc

SEQ ID NO: 22 - DNA, 28 bp, synthetic construct
  source 1..28: mol_type = other DNA
gctctagatctgacgctccgggaaagac

SEQ ID NO: 23 - DNA, 30 bp, synthetic construct
  source 1..30: mol_type = other DNA
gcggagctcatgcatcgcattaccatcctg

SEQ ID NO: 24 - DNA, 25 bp, synthetic construct
  source 1..25: mol_type = other DNA
gcggagctctcagcgctttgcagcc

SEQ ID NO: 25 - DNA, 31 bp, synthetic construct
  source 1..31: mol_type = other DNA
gcaccatgggccatcgcattaccatcctggg

SEQ ID NO: 26 - DNA, 24 bp, synthetic construct
  source 1..24: mol_type = other DNA
gcaaagcttcagcgctttgcagcc

SEQ ID NO: 27 - DNA, 40 bp, synthetic construct
  source 1..40: mol_type = other DNA
gcagatctgaattcaagaaaccaattgtccatattgcatc

SEQ ID NO: 28 - DNA, 36 bp, synthetic construct
  source 1..36: mol_type = other DNA
gctgaattcagatctgtgacgcagtagcggtaaacg

SEQ ID NO: 29 - DNA, 32 bp, synthetic construct
  source 1..32: mol_type = other DNA
catgccatgggtttgatgaccgtattttcttc

SEQ ID NO: 30 - DNA, 38 bp, synthetic construct
  source 1..38: mol_type = other DNA
gcagatctgcggccgctaggagataagactgcctcccg

SEQ ID NO: 31 - DNA, 27 bp, synthetic construct
  source 1..27: mol_type = other DNA
gcgaagcttaaggaggagaccccatgg

SEQ ID NO: 32 - DNA, 30 bp, synthetic construct
  source 1..30: mol_type = other DNA
gcggagctcatgggccatcgcattaccatc

SEQ ID NO: 33 - DNA, 35 bp, synthetic construct
  source 1..35: mol_type = other DNA
gcggagctcttattaatggtgatggtgatgatggc

SEQ ID NO: 34 - DNA, 5192 bp, unidentified
  source 1..5192: mol_type = unassigned DNA
atgatgaacgcacaaaaatcaaaaatcgcactgctgctcgcagcaagtgccgtcacaatggccttaaccggctgtggtggaagcgatggtaataacggcaatgatggtagtgatggtggtgagccagcaggtagcatccagacgttaaacctagatatcactaaagtaagctatgaaaatggtgcacctatggtcactgttttcgccactaacgaagccgacatgccagtgattggtctcgcaaatttagaaatcaaaaaagcactgcaattaataccggaaggggcgacaggcccaggtaatagcgctaactggcaaggcttaggctcatcaaagagctatgtcgataataaaaacggtagctatacctttaaattcgacgccttcgatagtaataaggtctttaatgctcaattaacgcaacgctttaacgttgtttctgctgcgggtaaattagcagacggaacgaccgttcccgttgccgaaatggttgaagatttcgacggccaaggtaatgcgccgcaatatacaaaaaatatcgttagccacgaagtatgtgcttcttgccacgtagaaggtgaaaagatttatcaccaagctactgaagtcgaaacttgtatttcttgccacactcaagagtttgcggatggtcgcggcaaaccccatgtcgcctttagtcacttaattcacaatgtgcataatgccaacaaagcttggggcaaagacaataaaatccctacagttgcacaaaatattgtccaagataattgccaagtttgtcacgttgaatccgacatgctcaccgaggcaaaaaactggtcacgtattccaacaatggaagtctgttctagctgtcacgtagacatcgattttgctgcgggtaaaggccactctcaacaactcgataactccaactgtatcgcctgccataacagcgactggactgctgagttacacacagccaaaaccaccgcaactaagaacttgattaatcaatacggtatcgagactacctcgacaattaataccgaaactaaagcagccacaattagtgttcaagttgtagatgcgaacggtactgctgttgatctcaagaccatcctgcctaaagtgcaacgcttagagatcatcaccaacgttggtcctaataatgcaaccttaggttatagtggcaaagattcaatatttgcaatcaaaaatggagctcttgatccaaaagctactatcaatgatgctggcaaactggtttataccactactaaagacctcaaacttggccaaaacggcgcagacagcgacacagcatttagctttgtaggttggtcaatgtgttctagcgaaggtaagtttgtagactgtgcagaccctgcatttgatggtgttgatgtaactaagtataccggcatgaaagcggatttagcctttgctactttgtcaggtaaagcaccaagtactcgccacgttgattctgttaacatgacagcctgtgccaattgccacactgctgagttcgaaattcacaaaggcaaacaacatgcaggctttgtgatgacagagcaactatcacacacccaagatgctaacggtaaagcgattgtaggccttgacgcatgtgtgacttgtcatactcctgatggcacctatagctttgccaaccgtggtgcgctagagctaaaactacacaaaaaacacgttgaagatgcctacggcctcattggtggcaattgtgcctcttgtcactcagacttcaaccttgagtctttcaagaagaaaggcgcattgaatactgccgctgcagcagataaaacaggtctatattctacgccgatcactgcaacttgtactacctgtcacacagttggcagccagtacatggtccatacgaaagaaaccctggagtctttcggtgcagttgttgatggcacaaaagatgatgctaccagtgcggcacagtcagaaacctgtttctactgccataccccaacagttgcagatcacactaaagtgaaaatgtaatttgcccaagcagggggagctcgctccccctttcttgaattttgttgggacaaattgggaagcctattatgaagaactgcctaaaaatgaaaaacctactgccggcacttaccatcacaatggcaatgtctgcagttatggcattagtcgtcacaccaaacgcttatgcgtcgaagtgggatgagaaaatgacgccagagcaagtcgaagccaccttagataagaagtttgccgaaggcaactactcccctaaaggcgccgattcttgcttgatgtgccataagaaatccgaaaaagtcatggaccttttcaaaggtgtccacggtgcgattgactcctctaagagtccaatggctggcctgcaatgtgaggcatgccacggcccactgggtcagcacaacaaaggcggcaacgagccgatgatcacttttggtaagcaatcaaccttaagtgccgacaagcaaaacagcgtatgtatgagctgtcaccaagacgataagcgtatgtcttggaatggcggtcaccatgacaatgccgatgttgcttgtgcttcttgtcaccaagtacacgtcgcaaaagatcctgtgttatctaaaaacacggaaatggaagtctgtactagctgccatacaaagcaaaaagcggatatgaataaacgctcaagtcacccactcaaatgggcacaaatgacctgtagcgactgtcacaatccccatgggagcatgacagattccgatcttaacaagcctagcgtgaatgatacctgttattcctgtcacgccgaaaaacgcggcccaaaactttgggagcatgcacccgtcactgagaattgtgtcacttgccacaatcctcacggtagtgtgaatgacggtatgctgaaaacccgtgcgccacagctatgtcagcaatgtcacgccagcgatggccacgccagcaacgcctacttaggtaacactggattaggttcaaatgtcggtgacaatgcctttactggtggaagaagctgcttaaattgccatagtcaggttcatggttctaaccatccatctggcaagctattacagcgctaaggagacgagaaaatgaaatttaaactcaatttgatcactctagcgttattagccaacacaggcttggccgtcgctgctgatggttatggtctagcgaatgccaatactgaaaaagtgaaattatccgcatggagctgtaaaggctgcgtcgttgaaacgggcacatcaggcactgtgggtgtcggtgtcggttataacagcgaagaggatattcgctctgccaatgcctttggtacatccaatgaagtggcgggtaaatttgatgccgatttaaactttaaaggtgaaaagggttatcgtgccagtgttgatgcttatcaactcggtatggatggcggtcgcttagatgtcaatgcgggcaaacaaggccagtacaacgtcaatgtgaactatcgccaaattgctacctacgacagcaatagcgccctatcgccctacgcgggtattggtggcaataacctcacgttaccggataactggataacagcaggttcaagcaaccaaatgccactcttgatggacagcctcaatgccctcgaactctcacttaaacgtgagcgcacggggttgggatttgaatatcaaggtgaatccctgtggagcacctatgttaactacatgcgtgaagagaaaaccggcttaaaacaagcctctggtagcttcttcaaccaatcgatgatgttagcagagccggtggattacaccactgacaccattgaagcgggtgtcaaactcaagggtgatcgttggtttaccgcactcagttacaatgggtcaatattcaaaaacgaatacaaccaattggactttgaaaatgcttttaaccccacctttggtgctcaaacccaaggtacgatggcactcgatccggataaccagtcacacaccgtgtcgctgatgggacagtacaacgatggcagcaacgcactgtcgggtcgtattctgaccggacaaatgagccaagatcaggcgttagtgacggataactaccgttatgctaatcagctcaataccgatgccgtcgatgccaaagtcgatctactgggtatgaacctgaaagtcgttagcaaagtgagcaatgatcttcgcttaacaggtagttacgattattacgaccgtgacaataatacccaagtagaagaatggactcagatcagcatcaacaatgtcaacggtaaggtggcttataacaccccttacgataatcgtacgcaacgctttaaagttgccgcagattatcgcattacccgcgatatcaaactcgatggtggttatgacttcaaacgtgaccaacgtgattatcaagaccgtgaaaccacggatgaaaataccgtttgggcccgtttacgtgtaaacagcttcgatacttgggacatgtgggtaaaaggcagttacggtaaccgtgacggctcacaataccaagcgtctgaatggacctcttctgaaaccaacagcctgttacgtaagtacaatctggctgaccgtgacagaactcaagtcgaagcacggatcacccattcgccattagaaagcctgactatcgatgttggtgcccgttacgcgttagatgattataccgatactgtgattggattaactgagtcaaaagacaccagttatgatgccaacatcagttatatgatcaccgctgacttactggcaaccgccttctacaattaccaaaccattgagtctgaacaggcgggtagcagcaattacagcaccccaacgtggacaggctttatagaagatcaggtagatgtggtcggtgcaggtatcagctacaacaatctgctggagaacaagttacgcctaggactggactacacctattccaactccgacagtaacactcaagtcagacaaggtatcactggcgactatggtgattattttgccaaagtgcataacattaacttatacgctcaatatcaagccaccgagaaactcgcgctgcgcttcgattacaaaattgagaactataaggacaatgacgccgcaaatgatatcgccgttgatggcatttggaacgtcgtaggttttggtagtaacagccatgactacaccgcacaaatgctgatgctgagcatgagttacaaactctaa

SEQ ID NO: 35 - DNA, 791 bp, unidentified
  source 1..791: mol_type = unassigned DNA
gtgcaacgttatggattgatggcgcttttgctcgtttcctgctgggccagcgcgcataacatcgtcatcgggcagccccttccgtcggtttttattgcggataaaggtgaaatgcggctggatggcggcaaggttaactatcaaaaatggaacagcctgtctcttccgggtcggacacgtttagttattcatgttgcaggacgattgtcggccaaagagcagtccgccccgcttattgcggccctgcagcgcgccaacctgccacaagaccggttccagaccacaaccatcgtgaatacagatgatgctttgcctggcagcagtctgtttgtgattaacagtatccgctccagtaaaaaagcctcaccatggcaacaatttattatcgacagtagcggcgtggcacaacatcgctggcagcttaagccagaaggtgccgctgtcatcgtgctggaccctgatggtcaggtaaagtttgcgaaagacacggcgctcagtgcggatgatgtttctcaggtcattgcaacattgcgtgcgctggcaggctgatcctggcaacccggtaaaggtaccgcacggtctgccaatccgacggaggtttatgaattttccaccttttccacaagctcaactagtattaacgatgtggatttagcaaaaaaaacctgtaaccctaaatgtaaaataacgggtaagcctgccaaccatgttatggcagattaagcgtctttttgaagggcaccgcatctttcgcgttgccgtaaatgtatccgtttataaggacagcccga

(19) United States
(12) Patent Application Publication
(10) Pub. No.: US 2026/0168116 A1
(43) Pub. Date: Jun. 18, 2026

(54) Engineering of a Photoautotrophic Cell for CO2 Fixation

(71) Applicant: Oxford University Innovation Limited, Oxford (GB)

(72) Inventors: Wei HUANG, Oxford (GB); Weiming TU, Oxford (GB)

(21) Appl. No.: 19/112,935

(22) PCT Filed: Sep. 21, 2023
(86) PCT No.: PCT/GB2023/052452

§ 371 Date: Mar. 18, 2025

(30) Foreign Application Priority Data
Sep. 21, 2022 (GB) 2213802.8
May 26, 2023 (GB) 2307916.3

(51) Int. Cl. C25B 9/50; C12N 9/02; C12N 9/88; C12N 15/52; C12N 15/74; C25B 3/21; C25B 3/26; C25B 9/19

(52) U.S. Cl. CPC C25B 9/50; C12N 9/0069; C12N 9/88; C12N 15/52; C12N 15/74; C12Y 113/11065; C12Y 402/01001; C25B 3/21; C25B 3/26; C25B 9/19; C12N 2800/101

(57) ABSTRACT

The invention relates to an electromicrobial system for photoautotrophic CO₂ fixation, comprising: A) a recombinantly engineered bacterial cell modified for photoautotrophic CO₂ fixation, wherein the bacterial cell comprises membrane-bound rhodopsin molecules and components for a CO₂ fixation pathway; and B) an electron source for donation of electrons into the electron transport chain; and related methods, uses and products.

The present invention relates to an electromicrobial system for photoautotrophic CO₂ fixation.

One vital challenge in the 21^{st} century is the sustainable production of chemicals and fuels from CO₂, using a biocatalyst and driven by a renewable energy source, for example from sunlight (1, 2). Harnessing the biological fixation of CO₂ for sequestering biomass is an ideal outcome in terms of mitigating rising levels of atmospheric CO₂, even more so if useful products such as chemicals and fuels could be generated. A recent important advance in synthetic biology has demonstrated that Escherichia coli can be genetically engineered to be converted from heterotrophy to autotrophy (3). Other approaches harnessed the native CO₂ fixation pathway of the chemolithotroph Ralstonia eutropha H16, coupled to an electrochemical supply of the required reducing power (4, 5). ‘Artificial leaf’ technology in a bioreactor containing R. eutropha, inspired by the Photosystem II complex in plants and cyanobacteria, can split water into H₂ and O₂, then use them to produce biomass and alcohols (6).

The engineering of photoautotrophic growth into a synthetic biology chassis would represent the next level of metabolic engineering, in terms of using synthetic biology for sustainable production of biomass, chemicals and fuels. Engineering photoautotropy would require exploitation of one of the light-driven systems that have naturally evolved, which are based on either chlorophyll or rhodopsins (7-9). Chlorophyll-based photosynthesis requires a large and relatively complex network of components for light-harvesting, charge-separation, then electron and proton transfers, for driving CO₂ fixation in cells. In contrast, rhodopsin-based utilisation of light is simpler, involving only one membrane protein (10) that usually comprises seven transmembrane a-helices. Microbial rhodopsins are widespread amongst the microbial inhabitants of sunny environments such as the upper ocean (11), and their functions have been dissected by heterologous production in E. coli (8, 12, 13). These studies demonstrated that retinal-binding PR generates a proton gradient that can be used for the production of biologically available energy in the form of ATP (12, 14). Although microbial rhodopsins have been reported as major contributors to the solar energy capture in the sea (9), there is no report of rhodopsin-driven autotrophic growth in microbes, likely due to the lack of electron donors required for reductive assimilation of CO_{2.}

There is thus a need to develop an alternative or improved methods and materials for microbial fixation of CO₂ and organic product production.

According to a first aspect of the invention there is provided an electromicrobial system for photoautotrophic CO₂ fixation, comprising:
A) a recombinantly engineered bacterial cell modified for photoautotrophic CO₂ fixation, wherein the bacterial cell comprises membrane-bound rhodopsin molecules and components for a CO₂ fixation pathway that together enable the biosynthesis of organic molecules from CO₂; and
B) an electron source for donation of electrons into the electron transport chain.

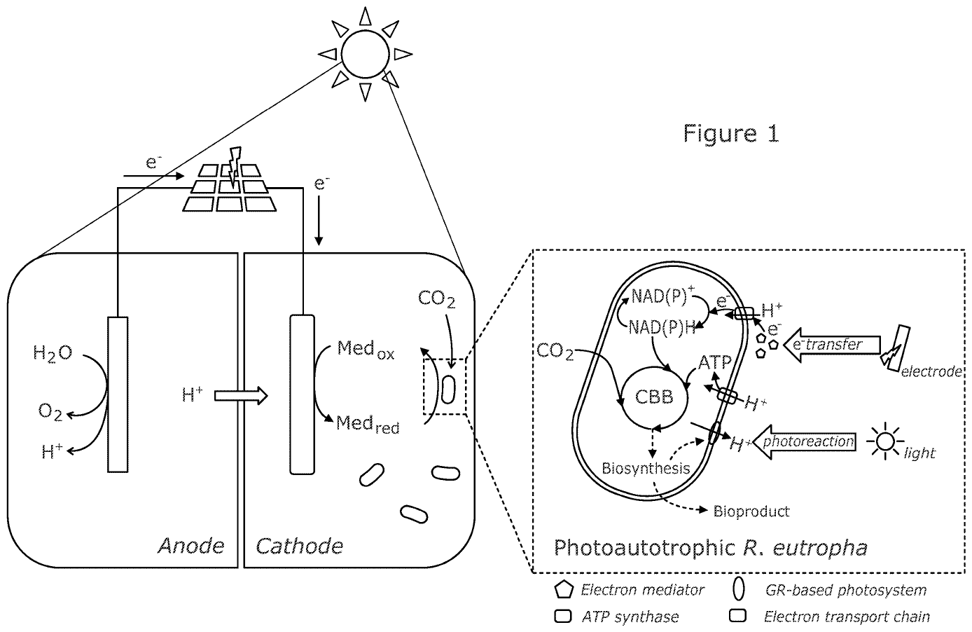

The present invention advantageously provides a light-powered electromicrobial system for CO₂ fixation. The invention shows that a closed redox loop can be constructed by integrating rhodopsin with an electron donor. If the electron donor can be supplied by an electrode powered by solar panel, a rhodopsin-based photo-electrosynthetic system could drive autotrophic growth of bacteria using CO₂ as the sole carbon source, and with light as the only energy input. This bioenergetic system has only two inputs, light and CO₂. The bioenergy for growth is supplied by installing Gloeobacter violaceus rhodopsin (GR), augmented by an external photocell that serves as the electron donor (FIG. 1). This engineered bacterium performs a hybrid form of photoautotrophy, which uses light to convert CO₂ into biomass.

### The Rhodopsin

The rhodopsin functions as a light-driven H⁺ ion transporter, which pumps protons from intracellular side of the membrane to the extracellular side. The resulting proton-motive force (PMF) is used by the ATP synthase to generate adenosine triphosphate (ATP) to power cell metabolism. The PMF is also used to drive the formation of NADPH, which is essential for CO₂ fixation pathways such as the Calvin cycle.

Rhodopsin (which may also be known as microbial rhodopsin) is a simple light-driven proton pump found broadly distributed in nature and it can also be easily engineered into different bacterial hosts.

The rhodopsin may comprise a recombinant rhodopsin. In one embodiment, the rhodopsin is a bacteriorhodopsin. The rhodopsin may comprise a proteorhodopsin (PR) or Gloeobacter rhodopsin (GR). In a preferred embodiment, the recombinant proton pump is the Gloeobacter violaceus rhodopsin.

Gloeobacter violaeus rhodopsin is originally from thylakoid-less cyanobacterium Gloeobacter violaceus PCC7421 (38) and advantageously has a high efficiency of proton pumping and a rapid photocycle. Furthermore, Gloeobacter violaceus rhodopsin has been shown to combine with other retinal analogues to absorb near-infrared light (850-950 nm) (42), which can significantly extend the light-harvesting spectrum and maximise energy harvesting per surface area (43). Gloeobacter violaceus rhodopsin has a pKa=˜4.8 (34), compared to proteorhodopsin which has a pKa=˜7.5 (35), Gloeobacter violaceus rhodopsin is functional at a lower pH, a situation often found in R. eutropha H16 growing using formate or CO₂ as the sole carbon source.

The rhodopsin may comprise the amino acid sequence of

(SEQ ID NO: 1)
MGLMTVFSSAPELALLGSTFAQVDPSNLSVSDSLTYGQFNLVYNA
FSFAIAAMFASALFFFSAQALVGQRYRLALLVSAIVVSIAGYHYF
RIFNSWDAAYVLENGVYSLTSEKFNDAYRYVDWLLTVPLLLVETV
AVLTLPAKEARPLLIKLTVASVLMIATGYPGEISDDITTRIIWGT
VSTIPFAYILYVLWVELSRSLVRQPAAVQTLVRNMRWLLLLSWGV
YPIAYLLPMLGVSGTSAAVGVQVGYTIADVLAKPVFGLLVFAIAL
VKTKADQESSEPHAAIGAAANKSGGSLIS.
(Gloeobacter violaceus rhodopsin)

For function, rhodopsin requires post-translational modification with a covalently conjugated retinal molecule. The bacterial cell may be engineered such that it is capable of retinal biosynthesis for functional rhodopsin. Additionally or alternatively, the bacterial cell may be provided with retinal, for example by supplementation and/or co-culture with a retinal producing organism.

The bacterial cell may be engineered such that it is capable of B-carotene biosynthesis. The engineered bacterial cell may be further provided with the expression of β-carotene 15, 15′-dioxygenase for conversion of β-carotene into retinal. In an embodiment wherein the bacterial cell comprises R. eutropha, the R. eutropha may be engineered such that it is capable of β-carotene biosynthesis. The engineered R. eutropha may be further provided with the expression of β-carotene 15, 15′-dioxygenase for conversion of β-carotene into retinal.

The bacterial cell, such as R. eutropha, may be engineered by the transformation with one or more, or all, of the genes selected from dxs, dxr, ispH, ispA, crtE, crtB, crtI, crtY, blh; or alternative genes encoding equivalent functioning enzymes. The genes may be provided with a promoter, such as their native promoter.

The bacterial cell may be engineered to express one or more, or all, of the enzymes selected from 1-deoxy-D-xylulose-5-phostaphate synthase (e.g. encoded by dxs), 1-deoxy-D-xylulose 5-phosphate reductoisomerase (e.g. encoded by dxr), 4-hydroxy-3-methylbut-2-enyl diphosphate reductase (e.g. encoded by ispH), farnesyl diphosphate synthase (e.g. encoded by ispA), geranylgeranyl diphosphate synthase (e.g. encoded by crtE), phytoene synthase (e.g. encoded by crtB), phytoene desaturase (e.g. encoded by crtI), lycopene cyclase (e.g. encoded by crtY), and β-carotene 15, 15′-dioxygenase (e.g. encoded by blh).

The bacterial cell may be engineered to express one or more, or all, of the enzymes selected from 1-deoxy-D-xylulose-5-phosphate synthase (e.g. encoded by dxs), 1-deoxy-D-xylulose 5-phosphate reductoisomerase (e.g. encoded by dxr), 4-hydroxy-3-methylbut-2-enyl diphosphate reductase (e.g. encoded by ispH), farnesyl diphosphate synthase (e.g. encoded by ispA), geranylgeranyl diphosphate synthase (e.g. encoded by crtE), phytoene synthase (e.g. encoded by crtB), phytoene desaturase (e.g. encoded by crtI), lycopene cyclase (e.g. encoded by crtY), and further engineered to express β-carotene 15, 15′-dioxygenase (e.g. encoded by blh) and a rhodopsin, such as Gleobacter violaeus rhodopsin.

The bacterial cell may be engineered to express one or more, or all, of the enzymes selected from 1-deoxy-D-xylulose-5-phostaphate synthase (e.g. encoded by dxs), 1-deoxy-D-xylulose 5-phosphate reductoisomerase (e.g. encoded by dxr), 4-hydroxy-3-methylbut-2-enyl diphosphate reductase (e.g. encoded by ispH), farnesyl diphosphate synthase (e.g. encoded by ispA), geranylgeranyl diphosphate synthase (e.g. encoded by crtE), phytoene synthase (e.g. encoded by crtB), phytoene desaturase (e.g. encoded by crtI), lycopene cyclase (e.g. encoded by crtY), and β-carotene 15, 15′-dioxygenase (e.g. encoded by blh); and further engineered to express a rhodopsin, such as Gloeobacter violaceus rhodopsin. The expression could be a combination of native expression and recombinant expression, or it may be entirely recombinant.

The bacterial cell, such as R. eutropha, may be engineered by the transformation with a four gene carotenoid biosynthetic pathway comprising crtI, crtY, crtE and crtB and a gene encoding β-carotene 15, 15′-dioxygenase (such as blh). The bacterial cell, such as R. eutropha, may be engineered by the transformation with crtI and crtY, and the function of endogenous crtE and crtB genes may be restored, for example by insertion of a promoter and/or recombinant crtE and crtB genes. One or more of the crtI, crtY, crtE and crtB genes may be from Erwinia herbicola (Pantoea agglomerans) or E. coli.

The skilled person will recognise that expression of one or more pathway components may be a combination of native expression and recombinant expression, or it may be entirely recombinant.

Equivalent genes may be provided from other strains or species. In particular, the bacterial cell may be engineered to provide the carotenoid biosynthetic pathway using any suitable genes for the expression of one or more of 1-deoxy-D-xylulose 5-phosphate reductoisomerase (e.g. encoded by dxr); geranylgeranyl diphosphate synthase (e.g. encoded by crtE); zeaxanthin glucosyltransferase (e.g. encoded by crtX); phytoene synthase (e.g. encoded by crtB); phytoene desaturase (e.g. encoded by crtI); lycopene cyclase (e.g. encoded by crtY).

The phytoene desaturase may comprise the amino acid sequence of

(SEQ ID NO: 2)
MKPTTVIGAGFGGLALAIRLQAAGIPVLLLEQRDKPGGRAYVYED
QGFTFDAGPTVITDPSAIEELFALAGKQLKEYVELLPVTPFYRLC
WESGKVFNYDNDQTRLEAQIQQFNPRDVEGYRQFLDYSRAVFKEG
YLKLGTVPFLSFRDMLRAAPQLAKLQAWRSVYSKVASYIEDEHLR
QAFSFHSLLVGGNPFATSSIYTLIHALEREWGVWFPRGGTGALVQ
GMIKLFQDLGGEVVLNARVSHMETTGNKIEAVHLEDGRRFLTQAV
ASNADVVHTYRDLLSQHPAAVKQSNKLQTKRMSNSLFVLYFGLNH
HHDQLAHHTVCFGPRYRELIDEIFNHDGLAEDFSLYLHAPCVTDS
SLAPEGCGSYYVLAPVPHLGTANLDWTVEGPKLRDRIFAYLEQHY
MPGLRSQLVTHRMFTPFDFRDQLNAYHGSAFSVEPVLTQSAWFRP
HNRDKTITNLYLVGAGTHPGAGIPGVIGSAKATAGLMLEDLI.
(CrtI)

The lycopene cyclase may comprise the amino acid sequence of

(SEQ ID NO: 3)
MQPHYDLILVGAGLANGLIALRLQQQQPDMRILLIDAAPQAGGNHTWSFH
HDDLTESQHRWIAPLVVHHWPDYQVRFPTRRRKLNSGYFCITSQRFAEVL
QRQFGPHLWMDTAVAEVNAESVRLKKGQVIGARAVIDGRGYAANSALSVG
FQAFIGQEWRLSHPHGLSSPIIMDATVDQQNGYRFVYSLPLSPTRLLIED
THYIDNATLDPECARQNICDYAAQQGWQLQTLLREEQGALPITLSGNADA
FWQQRPLACSGLRAGLFHPTTGYSLPLAVAVADRLSALDVFTSASIHHAI
THFARERWQQQGFFRMLNRMLFLAGPADSRWRVMQRFYGLPEDLIARFYA
GKLTLTDRLRILSGKPPVPVLAALQAIMTTHR.
(CrtY)

The geranylgeranyl diphosphate synthase may comprise the amino acid sequence of

(SEQ ID NO: 4)
MTVCAKKHVHLTRDAAEQLLADIDRRLDQLLPVEGERDVVGAAMREGALA
PGKRIRPMLLLLTARDLGCAVSHDGLLDLACAVEMVHAASLILDDMPCMD
DAKLRRGRPTIHSHYGEHVAILAAVALLSKAFGVIADADGLTPLAKNRAV
SELSNAIGMQGLVQGQFKDLSEGDKPRSAEAILMTNHFKTSTLFCASMQM
ASIVANASSEARDCLHRFSLDLGQAFQLLDDLTDGMTDTGKDSNQDAGKS
TLVNLLGPRAVEERLRQHLQLASEHLSAACQHGHATQHFIQAWFDKKLAA
VS.
(CrtE)

The phytoene synthase may comprise the amino acid sequence of

(SEQ ID NO: 5)
MNNPSLLNHAVETMAVGSKSFATASKLFDAKTRRSVLMLYAWCRHCDDVI
DDQTLGFQARQPALQTPEQRLMQLEMKTRQAYAGSQMHEPAFAAFQEVAM
AHDIAPAYAFDHLEGFAMDVREAQYSQLDDTLRYCYHVAGVVGLMMAQIM
GVRDNATLDRACDLGLAFQLTNIARDIVDDAHAGRCYLPASWLEHEGLNK
ENYAAPENRQALSRIARRLVQEAEPYYLSATAGLAGLPLRSAWAIATAKQ
VYRKIGVKVEQAGQQAWDQRQSTTTPEKLTLLLAASGQALTSRMRAHPPR
PAHLWQRPL.
(CrtB)

The 1-deoxy-D-xylulose-5-phostaphate synthase (e.g. encoded by dxs) may comprise the amino acid sequence of

(SEQ ID NO: 6)
MHRITILGATGSIGESTLDVVRRHADRYVVHALTAHRQVRKLADQCVEFR
PARAVVGTAEAALELETLLRDAGVKTEVSHGEAALESVAADAQTDSVMAA
IVGAAGLRPTLAAARAGKRVLLANKEALVMSGRIFMDAVREHGATLLPID
SEHNAIFQCLPADDPRYGRGVARVLLTASGGPFRTRDPATLHDISPDQAC
AHPNWVMGRKISVDSATMMNKGLEVIEAHWLFGAPAERIEVLIHPQSIVH
SMVAYTDGSVLAQLGNPDMRTPIAYGLAYPERIDAGVTPLDLTVAGGLHF
EKPDLVRFPCLGLAFDALRAGGVAPAALNAANEVAVEAFLGGTVRFTDIA
GIVRQVLEATPQGPADTLEAVLSADALAREAAREGVAALAAKR.
(DXS)

The 4-hydroxy-3-methylbut-2-enyl diphosphate reductase (e.g. encoded by ispH) may comprise the amino acid sequence of

(SEQ ID NO: 7)
MAQPRGFCAGVDRAIEIVERALERFGAPIYVRHEIVHNAYVVAGLRRKGA
VFVRELDEVPAGATVIFSAHGVSREVRADAAARGLHVFDATCPLVTKVHV
EVSKMRAEGCEIVMIGHRGHPEVEGTMGQASSGMLLVESVADVATLQVTD
PSRLAYVTQTTLSVDETREIVAALKARFPQIREPKKQDICYATQNRQDAV
KFMAPQVEVVIVVGSPNSSNSNRLRELAERLGVPAYMVDAPEQVRPEWIA
GKRRIGLTAGASAPEALAQSIVERLRELGASQVRPLDGIEENMAFPLPRG
LLPASAAA.
(IspH)

The farnesyl diphosphate synthase (e.g. encoded by ispA) may comprise the amino acid sequence of

(SEQ ID NO: 8)
MSDFAQWMQAQGARTEAALQAALPAAETVPHTLHEAMRYAALSGGKRVRP
LLVHAAGEVSGAAPAACDAAACAVEMIHAYSLVHDDMPCMDDDDLRRGRP
TVHKAYDEATALLVGDALQTQAFIVLAGAGAIAPAARLQLVAELALASGS
TGMAGGQAIDLQNVGRAMTREALEAMHRMKTGALLRASVRMGALCGEIDA
EGLAALDRYAAAVGLAFQVVDDILDVTADTATLGKTAGKDAAHDKPTYVS
LMGLDPARALAGTLRADAHEALAGFGERADRLRDLADLIVLRTH.
(IspA)

The bacterial cell, such as R. eutropha, may be transformed with the Erwinia uredovora (Pantoea ananatis) crtEXYIB operon, preferably with its promoter.

The crtEXYIB operon and promoter may comprise the nucleotide sequence of

(SEQ ID NO: 9)
GTGCAACGTTATGGATTGATGGCGCTTTTGcTCGTTTCCTGCTGGGCCAG
CGCGCATAACATCGTCATCGGGCAGCCCcTTCCGTCGGTTTTTATTGCGG
ATAAAGGTGAAATGCGGCTGGATGGCGGCAAGGTTAACTATCAAAAAtgG
AACAGCCTGTCTCTTCCGGgTCGGACACGTTTAGTTATTCATGTTGCAGG
ACGATTGTCGGCCAAAGAGCAGTCCGCCCCGCTTATTGCGGCCCTGCAGC
GCGCCAACCTGCCACAAGACCGGTTCCAGACCACAACCATCGTGAATACA
GATGATGCTTTGCCTGGCAGCAGTCTGTTTGTGATTAACAGTATCCGCTC
CAGTAAAAAAGCCTCACCATGGCAACAATTTATTATCGACAGTAGCGGCG
TGGCACAACATCGCTGGCAGCTTAAGCCAGAAGGTGCCGCTGTCATCGTG
CTGGACCCTGATGGTCAGGTAAAGTTTGCGAAAGACACGGCGCTCAGTGC
GGATGATGTTTCTCAGGTCATTGCAACATTGCGTGCGCTGGCAGGCTGAT
CCTGGCAACCCGGTAAAGGTACCGCACGGTCTGCCAATCCGACGGAGGTT
TATGAATTTTCCACCTTTTCCACAAGCTCAACTAGTATTAACGATGTGGA
TTTAGCAAAAAAAACCTGTAACCCTAAATGTAAAATAACGGGTAAGCCTG
CCAACCATGTTATGGCAGATTAAGCGTCTTTTTGAAGGGCACCGCATCTT
TCGCGTTGCCGTAAATGTATCCGTTTATAAGGACAGCCCGAATGACGGTC
TGCGCAAAAAAACACGTTCATCTCACTCGCGATGCTGCGGAGCAGTTACT
GGCTGATATTGATCGACGCCTTGATCAGTTATTGCCCGTGGAGGGAGAAC
GGGATGTTGTGGGTGCCGCGATGCGTGAAGGTGCGCTGGCACCGGGAAAA
CGTATTCGCCCCATGTTGCTGTTGCTGACCGCCCGCGATCTGGGTTGCGC
TGTCAGCCATGACGGATTACTGGATTTGGCCTGTGCGGTGGAAATGGTCC
ACGCGGCTTCGCTGATCCTTGACGATATGCCCTGCATGGACGATGCGAAG
CTGCGGCGCGGACGCCCTACCATTCATTCTCATTACGGAGAGCATGTGGC
AATACTGGCGGCGGTTGCCTTGCTGAGTAAAGCCTTTGGCGTAATTGCCG
ATGCAGATGGCCTCACGCCGCTGGCAAAAAATCGGGCGGTTTCTGAACTG
TCAAACGCCATCGGCATGCAAGGATTGGTTCAGGGTCAGTTCAAGGATCT
GTCTGAAGGGGATAAGCCGCGCAGCGCTGAAGCTATTTTGATGACGAATC
ACTTTAAAACCAGCACGCTGTTTTGTGCCTCCATGCAGATGGCCTCGATT
GTTGCGAATGCCTCCAGCGAAGCGCGTGATTGCCTGCATCGTTTTTCACT
TGATCTTGGTCAGGCATTTCAACTGCTGGACGATTTGACCGATGGCATGA
CCGACACCGGTAAGGATAGCAATCAGGACGCCGGTAAAATCGACGCTGGT
CAATCTGTTAGGCCCGAGGGCGGTTGAAGAACGTCTGAGACAACATCTTC
AGCTTGCCAGTGAGCATCTCTCTGCGGCCTGCCAACACGGGCACGCCACT
CAACATTTTATTCAGGCCTGGTTTGACAAAAAACTCGCTGCCGTCAGTTA
AGGATGCTGCATGAGCCATTTCGCGGCGATCGCACCGCCTTTTTACAGCC
ATGTTCGCGCATTACAGAATCTCGCTCAGGAACTGGTCGCGCGCGGTCAT
CGGGTGACCTTTATTCAGCAATACGATATTAAACACTTGATCGATAGCGA
AACCATTGGATTTCATTCCGTCGGGACAGACAGCCATCCCCCCGGCGCGT
TAACGCGCGTGCTACACCTGGCGGCTCATCCTCTGGGGCCGTCAATGCTG
AAGCTCATCAATGAAATGGCGCGCACCACCGATATGCTGTGCCGCGAACT
CCCCCAGGCATTTAACGATCTGGCCGTCGATGGCGTCATTGTTGATCAAA
TGGAACCGGCAGGCGCGCTCGTTGCTGAAGCACTGGGACTGCCGTTTATC
TCTGTCGCCTGCGCGCTGCCTCTCAATCGTGAACCGGATATGCCCCTGGC
GGTTATGCCTTTCGAATACGGGACCAGCGACGCGGCTCGCGAACGTTATG
CCGCCAGTGAAAAAATTTATGACTGGCTAATGCGTCGTCATGACCGTGTC
ATTGCCGAACACAGCCACAGAATGGGCTTAGCCCCCCCGGCAAAAGCTTC
ACCAGTGTTTTTCGCCACTGGCGCAAATCAGCCAGCTTGTTCCTGAACTG
GATTTTCCCCGCAAAGCGTTACCGGCTTGTTTTCATGCCGTCGGGCCTCT
GCGCGAAACGCACGCACCGTCAACGTCTTCATCCCGTTATTTTACATCCT
CAGAAAAACCCCGGATTTTCGCCTCGCTGGGCACGCTTCAGGGACACCGT
TATGGGCTGTTTAAAACGATAGTGAAAGCCTGTGAAGAAATTGACGGTCA
GCTCCTGTTAGCCCACTGTGGTCGTCTTACGGACTCTCAGTGTGAAGAGC
TGGCGCGAAGCCGTCATACACAGGTGGTGGATTTTGCCGATCAGTCAGCC
GCGCTGTCTCAGGCGCAGCTGGCGATCACCCACGGCGGCATGAATACGGT
ACTGGACGCGATTAATTACCGGACGCCCCTTTTAGCGCTTCCGCTGGCCT
TTGATCAGCCCGGCGTCGCGTCACGCATCGTTTATCACGGCATCGGCAAG
CGTGCTTCCCGCTTTACCACCAGCCATGCTTTGGCTCGTCAGATGCGTTC
ATTGCTGACCAACGTCGACTTTCAGCAGCGCATGGCGAAAATCCAGACAG
CCCTTCGTTTGGCAGGGGGCACCATGGCCGCTGCCGATATCATTGAGCAG
GTTATGTGCACCGGTCAGCCTGTCTTAAGTGGGAGCGGCTATGCAACCGC
ATTATGATCTGATTCTCGTGGGGGCTGGACTCGCGAATGGCCTTATCGCC
CTGCGTCTTCAGCAGCAGCAACCTGATATGCGTATTTTGCTTATCGACGC
CGCACCCCAGGCGGGCGGGAATCATACGTGGTCATTTCACCACGATGATT
TGACTGAGAGCCAACATCGTTGGATAGCTCCGCTGGTGGTTCATCACTGG
CCCGACTATCAGGTACGCTTTCCCACACGCCGTCGTAAGCTGAACAGCGG
CTACTTTTGTATTACTTCTCAGCGTTTCGCTGAGGTTTTACAGCGACAGT
TTGGCCCGCACTTGTGGATGGATACCGCGGTCGCAGAGGTTAATGCGGAA
TCTGTTCGGTTGAAAAAGGGTCAGGTTATCGGTGCCCGCGCGGTGATTGA
CGGGCGGGGTTATGCGGCAAATTCAGCACTGAGCGTGGGCTTCCAGGCGT
TTATTGGCCAGGAATGGCGATTGAGCCACCCGCATGGTTTATCGTCTCCC
ATTATCATGGATGCCACGGTCGATCAGCAAAATGGTTATCGCTTCGTGTA
CAGCCTGCCGCTCTCGCCGACCAGATTGTTAATTGAAGACACGCACTATA
TTGATAATGCGACATTAGATCCTGAATGCGCGCGGCAAAATATTTGCGAC
TATGCCGCGCAACAGGGTTGGCAGCTTCAGACACTGCTGCGAGAAGAACA
GGGCGCCTTACCCATTACTCTGTCGGGCAATGCCGACGCATTCTGGCAGC
AGCGCCCCCTGGCCTGTAGTGGATTACGTGCCGGTCTGTTCCATCCTACC
ACCGGCTATTCACTGCCGCTGGCGGTTGCCGTGGCCGACCGCCTGAGTGC
ACTTGATGTCTTTACGTCGGCCTCAATTCACCATGCCATTACGCATTTTG
CCCGCGAGCGCTGGCAGCAGCAGGGCTTTTTCCGCATGCTGAATCGCATG
CTGTTTTTAGCCGGACCCGCCGATTCACGCTGGCGGGTTATGCAGCGTTT
TTATGGTTTACCTGAAGATTTAATTGCCCGTTTTTATGCGGGAAAACTCA
CGCTGACCGATCGGCTACGTATTCTGAGCGGCAAGCCGCCTGTTCCGGTA
TTAGCAGCATTGCAAGCCATTATGACGACTCATCGTTAAAGAGCGACTAC
ATGAAACCAACTACGGTAATTGGTGCAGGCTTCGNTGGCCTGGCACTGGC
AATTCGTCTACAAGCTGCGGGGATCCCCGTCTTACTGCTTGAACAACGTG
ATAAACCCGGCGGTCGGGCTTATGTCTACGAGGATCAGGGGTTTACCTTT
GATGCAGGCCCGACGGTTATCACCGATCCCAGTGCCATTGAAGAACTGTT
TGCACTGGCAGGAAAACAGTTAAAAGAGTATGTCGAACTGCTGCCGGTTA
CGCCGTTTTACCGCCTGTGTTGGGAGTCAGGGAAGGTCTTTAATTACGAT
AACGATCAAACCCGGCTCGAAGCGCAGATTCAGCAGTTTAATCCCCGCGA
TGTCGAAGGTTATCGTCAGTTTCTGGACTATTCACGCGCGGTGTTTAAAG
AAGGCTATCTAAAGCTCGGTACTGTCCCTTTTTTATCGTTCAGAGACATG
CTTCGCGCCGCACCTCAACTGGCGAAACTGCAGGCATGGAGAAGCGTTTA
CAGTAAGGTTGCCAGTTACATCGAAGATGAACATCTGCGCCAGGCGTTTT
CTTTCCACTCGCTGTTGGTGGGCGGCAATCCCTTCGCCACCTCATCCATT
TATACGTTGATACACGCGCTGGAGCGTGAGTGGGGCGTCTGGTTTCCGCG
TGGCGGCACCGGCGCATTAGTTCAGGGGATGATAAAGCTGTTTCAGGATC
TGGGTGGCGAAGTCGTGTTAAACGCCAGAGTCAGCCATATGGAAACGACA
GGAAACAAGATTGAAGCCGTGCATTTAGAGGACGGTCGCAGGTTCCTGAC
GCAAGCCGTCGCGTCAAATGCAGATGTGGTTCATACCTATCGCGACCTGT
TAAGCCAGCACCCTGCCGCGGTTAAGCAGTCCAACAAACTGCAGACTAAG
CGCATGAGTAACTCTCTGTTTGTGCTCTATTTTGGTTTGAATCACCATCA
TGATCAGCTCGCGCATCACACGGTTTGTTTCGGCCCGCGTTACCGCGAGC
TGATTGACGAAATTTTTAATCATGATGGCCTCGCAGAGGACTTCTCACTT
TATCTGCACGCGCCCTGTGTCACGGATTCGTCACTGGCGCCTGAAGGTTG
CGGCAGTTACTATGTGTTGGCGCCGGTGCCGCATTTAGGCACCGCGAACC
TCGACTGGACGGTTGAGGGGCCAAAACTACGCGACCGTATTTTTGCGTAC
CTTGAGCAGCATTACATGCCTGGCTTACGGAGTCAGCTGGTCACGCACCG
GATGTTTACGCCGTTTGATTTTCGCGACCAGCTTAATGCCTATCATGGCT
CAGCCTTTTCTGTGGAGCCCGTTCTTACCCAGAGCGCCTGGTTTCGGCCG
CATAACCGCGATAAAACCATTACTAATCTCTACCTGGTCGGCGCAGGCAC
GCATCCCGGCGCAGGCATTCCTGGCGTCATCGGCTCGGCAAAAGCGACAG
CAGGTTTGATGCTGGAGGATCTGATATGAATAATCCGTCGTTACTCAATC
ATGCGGTCGAAACGATGGCAGTTGGCTCGAAAAGTTTTGCGACAGCCTCA
AAGTTATTTGATGCAAAAACCCGGCGCAGCGTACTGATGCTCTACGCCTG
GTGCCGCCATTGTGACGATGTTATTGACGATCAGACGCTGGGCTTTCAGG
CCCGGCAGCCTGCCTTACAAACGCCCGAACAACGTCTGATGCAACTTGAG
ATGAAAACGCGCCAGGCCTATGCAGGATCGCAGATGCACGAACCGGCGTT
TGCGGCTTTTCAGGAAGTGGCTATGGCTCATGATATCGCCCCGGCTTACG
CGTTTGATCATCTGGAAGGCTTCGCCATGGATGTACGCGAAGCGCAATAC
AGCCAACTGGATGATACGCTGCGCTATTGCTATCACGTTGCAGGCGTTGT
CGGCTTGATGATGGCGCAAATCATGGGCGTGCGGGATAACGCCACGCTGG
ACCGCGCCTGTGACCTTGGGCTGGCATTTCAGTTGACCAATATTGCTCGC
GATATTGTGGACGATGCGCATGCGGGCCGCTGTTATCTGCCGGCAAGCTG
GCTGGAGCATGAAGGTCTGAACAAAGAGAATTATGCGGCACCTGAAAACC
GTCAGGCGCTGAGCCGTATCGCCCGTCGTTTGGTGCAGGAAGCAGAACCT
TACTATTTGTCTGCCACAGCCGGCCTGGCAGGGTTGCCCCTGCGTTCCGC
CTGGGCAATCGCTACGGCGAAGCAGGTTTACCGGAAAATAGGTGTCAAAG
TTGAACAGGCCGGTCAGCAAGCCTAGGATCAGCGGCAGTCAACGACCACG
CCCGAAAAATTAACGCTGCTGCTGGCCGCCTCTGGTCAGGCCCTTACTCC
CGGATGCGGGCTCATCCTCCCCGCCCTGCGCATCTCTGGCAGCGCCCGCT
CTAGCGCCaTGTCTTTCCCGGAGCGTC.
(crtEXYIB operon)

In one embodiment, the crtX gene may not be provided.

Additionally, β-carotene 15, 15′-dioxygenase may be provided, which may be encoded by the blh gene. The blh gene may be from the uncultured marine bacterium 66A03.

The β-carotene 15, 15′-dioxygenase (encoded by the blh gene) may comprise the amino acid sequence of

(SEQ ID NO: 10)
MGGLMLIDWCALALVVFIGLPHGALDAAISFSMISSAKRIARLAGILLIY
LLLATAFFLIWYQLPAFSLLIFLLISIIHFGMADFNASPSKLKWPHIIAH
GGVVTVWLPLIQKNEVTKLFSILTNGPTPILWDILLIFFLCWSIGVCLHT
YETLRSKHYNIAFELIGLIFLAWYAPPLVTFATYFCFIHSRRHFSFVWKQ
LQHMSSKKMMIGSAIILSCTSWLIGGGIYFFLNSKMIASEAALQTVFIGL
AALTVPHMILIDFIFRPHSSRIKIKNKGELEGKPIPNPLLGLDSTRTGHH
HHHH.
(β-carotene 15, 15′-dioxygenase)

The bacterial cell, such as R. eutropha, may be further engineered for expression of 1-deoxy-D-xylulose 5-phosphate reductoisomerase, which may be by overexpression of the endogenous dxr gene or transformation with a recombinant dxr gene. Preferably, bacterial cell, such as R. eutropha, is transformed with a dxr gene for overexpression. Preferably, a promoter, such as P_{BAD}, is provided for overexpression.

The 1-deoxy-D-xylulose 5-phosphate reductoisomerase may comprise the amino acid sequence of

(SEQ ID NO: 11)
MHRITILGATGSIGESTLDVVRRHADRYVVHALTAHRQVRKLADQCVEFR
PARAVVGTAEAALELETLLRDAGVKTEVSHGEAALESVAADAQTDSVMAA
IVGAAGLRPTLAAARAGKRVLLANKEALVMSGRIFMDAVREHGATLLPID
SEHNAIFQCLPADDPRYGRGVARVLLTASGGPFRTRDPATLHDISPDQAC
AHPNWVMGRKISVDSATMMNKGLEVIEAHWLFGAPAERIEVLIHPQSIVH
SMVAYTDGSVLAQLGNPDMRTPIAYGLAYPERIDAGVTPLDLTVAGGLHF
EKPDLVRFPCLGLAFDALRAGGVAPAALNAANEVAVEAFLGGTVRFTDIA
GIVRQVLEATPQGPADTLEAVLSADALAREAAREGVAALAAKR.
(1-deoxy-D-xylulose 5-phosphate reductoisomerase)

The present invention has surprisingly identified that the wild-type dxr expression in R. eutropha is defective or not sufficient to convert 1-deoxy-D-xyulose-5-phosphate to 2-C-methyl-D-erythritol-4-phosphate, which is part of the retinal biosynthesis pathway. Restoring the 1-deoxy-D-xylulose 5-phosphate reductoisomerase expression by overexpression using a recombinant dxr gene and promoter provides an important component in the biosynthesis pathway of retinal.

The crt operon genes may be promoted by the crtE endogenous promoter (P_{crtE}), or an alternative promoter such as P_{BAD}. Other genes, such as one or more of the dxr, blh and rhodopsin genes (such as Gloeobacter violaceus rhodopsin (GR)) may be under the control of the arabinose inducible P_{BAD} promoter. The skilled person will recognise that any suitable promoter may be used.

The crtE endogenous promoter (P_{crtE}) may comprise the sequence of

SEQ ID NO: 11
GTGCAACGTTATGGATTGATGGCGCTTTTGcTCGTTTCCTGCTGGGCCAG
CGCGCATAACATCGTCATCGGGCAGCCCcTTCCGTCGGTTTTTATTGCGG
ATAAAGGTGAAATGCGGCTGGATGGCGGCAAGGTTAACTATCAAAAAtgG
AACAGCCTGTCTCTTCCGGgTCGGACACGTTTAGTTATTCATGTTGCAGG
ACGATTGTCGGCCAAAGAGCAGTCCGCCCCGCTTATTGCGGCCCTGCAGC
GCGCCAACCTGCCACAAGACCGGTTCCAGACCACAACCATCGTGAATACA
GATGATGCTTTGCCTGGCAGCAGTCTGTTTGTGATTAACAGTATCCGCTC
CAGTAAAAAAGCCTCACCATGGCAACAATTTATTATCGACAGTAGCGGCG
TGGCACAACATCGCTGGCAGCTTAAGCCAGAAGGTGCCGCTGTCATCGTG
CTGGACCCTGATGGTCAGGTAAAGTTTGCGAAAGACACGGCGCTCAGTGC
GGATGATGTTTCTCAGGTCATTGCAACATTGCGTGCGCTGGCAGGCTGAT
CCTGGCAACCCGGTAAAGGTACCGCACGGTCTGCCAATCCGACGGAGGTT
TATGAATTTTCCACCTTTTCCACAAGCTCAACTAGTATTAACGATGTGGA
TTTAGCAAAAAAAACCTGTAACCCTAAATGTAAAATAACGGGTAAGCCTG
CCAACCATGTTATGGCAGATTAAGCGTCTTTTTGAAGGGCACCGCATCTT
TCGCGTTGCCGTAAATGTATCCGTTTATAAGGACAGCCCGA.

The P_{BAD} promoter may comprise the sequence of

SEQ ID NO: 12
AAGAAACCAATTGTCCATATTGCATCAGACATTGCCGTCACTGCGTCTTT
TACTGGCTCTTCTCGCTAACCAAACCGGTAACCCCGCTTATTAAAAGCAT
TCTGTAACAAAGCGGGACCAAAGCCATGACAAAAACGCGTAACAAAAGTG
TCTATAATCACGGCAGAAAAGTCCACATTGATTATTTGCACGGCGTCACA
CTTTGCTATGCCATAGCATTTTTATCCATAAGATTAGCGGATCCTACCTG
ACGCTTTTTATCGCAACTCTCTACTGTTTCTCCAT.

In an embodiment wherein the bacterial cell encodes one or more of the required genes for the retinal biosynthetic pathway, the skilled person will recognise that there are several options for restoring the pathway. For example, defective or missing genes may be replaced by transformation with a recombinant gene from the same or different strain or species. The gene may be provided with or without a promoter, such as a strong promoter. Alternatively the expression of an endogenous gene may be restored or increased by the insertion of a promoter for such a gene. Furthermore, a defective gene may be restored by one or more restorative mutations, such as substitutions, deletions or additions.

If the bacterial cell already expresses one or more functional enzymes of the retinal biosynthesis pathway, the bacterial cell may only be engineered to express the enzymes that ae missing from the pathway. Additionally or alternatively, one or more intermediate molecules in the pathway may be supplied in the media by supplement or a co-cultured cell.

Preferably the transformed genes are chromosomally (i.e. stably) integrated. Preferably, one or more, or all, of the transformed genes are codon optimised for expression in the bacterial cell, such as R. eutropha.

### The Bacterial Cell

The bacterial cell may comprise any bacterial cell that has, or is engineered to provide, a rhodopsin and optionally components for a CO₂ fixation pathway.

In a preferred embodiment, the bacterial cell is Gram negative. The bacterial cell may comprise Ralstonia spp, such as Ralstonia eutropha. Alternatively, the bacterial cell may comprise E. coli, Pseudomonas spp, such as Pseudomonas putida, or Synechocystis spp., such as Synechocystis sp. PCC6803. In one embodiment, the phaCAB operon of the Ralstonia spp. may be knocked out. In one embodiment, the bacterial cell comprises R. eutropha H16 (ATCC 17699) or its derivative strain RHM5 (H16Δpha).

Advantageously, Ralstonia eutropha has a native CO₂ fixation pathway, which is encoded on two operons of the Calvin-Benson-Bassham (CBB) cycle, one on chromosome 2 and the other on its pHG1 megaplasmid. Further advantageously, the phaCAB operon of the Ralstonia spp. may be knocked out to maximise carbon flux towards biomass.

The bacterial cell may comprise Ralstonia eutropha that is engineered to provide a rhodopsin and a retinal biosynthesis pathway.

The retinal biosynthesis pathway of Ralstonia eutropha may be provided by transforming Ralstonia eutropha with the genes of dxr, crtI, crtY, crtE and crtB and a gene for expressing β-carotene 15, 15′-dioxygenase, such as blh. Preferably one or more promoters may be provided for expression of such genes.

In another embodiment, the bacterial cell may be a simple cell that has, or has been engineered to provide a rhodopsin, and optionally components for a CO₂ fixation pathway.

The simple cell (which may also be known as a “chromosome-free bacterial cell”) may be in accordance with patent application publication WO2021079145A1, which is herein incorporated by reference. The skilled person will recognise that chromosome-free bacterial cells are safe and programmable platforms for synthetic biology, for example as described by Fan et al. (Proc. Natl Acad. Sci. 117, 6752-6761. 2020), which is herein incorporated by reference.

The skilled person will recognise that a given bacterial cell or simple cell may already have some of the required components for the photoautotrophic growth and CO₂ fixation according to the invention. Therefore, the engineering of such a cell may comprise only the transformation or modification of the necessary genetic information to provide, or activate, the expression of the components that are not already encoded and/or expressed in the cell.

### The Electron Source

The electron source may be any system that is capable of donating electrons into the electron transport chain directly or through intermediate molecules such as an electron mediator.

The electron source may comprise an electrode linked to a source of electricity. The source of electricity may be a solar panel, wind turbine, hydroelectric turbine, nuclear fission or fusion. Preferably the source of electricity is from a renewable energy source (i.e. not via the burning of fossil fuels). The electron source may comprise a potentiostat to control the cathode potential and/or a voltage regulator. A voltage regulator can help stabilise the cathode potential to avoid cathode-potential fluctuations that could harm cells.

In a particularly preferred embodiment, the source of electricity is a solar panel. The solar panel may otherwise be described as an “external photocell”. The solar panel may be electrically connected to an electrode, which can donate electrons to the electron transport chain. The electrons may be mediated via an electron mediator, such as riboflavin.

Advantageously, the use of solar panels for the electron source complements the need for the photoautotrophic bacterial cell to utilise a light source for the rhodopsin (proton pump). Therefore, light energy can provide energy for both functions of driving the rhodopsin proton pump and feeding electrons into the electron transport chain. In particular, the system may operate from just light energy and CO₂ as a carbon source.

The electrode may be part of an electrolytic cell or galvanic cell. The electrode may be a cathode linked to a power source and wherein the cathode is in fluid contact with the bacterial cell. The cathode may be in the same media and/or chamber as the bacterial cell. The anode and cathode may be separated by a barrier, which may comprise a proton exchange membrane. In a preferred embodiment, a dual chamber electrode system is provided, which has a membrane only allowing proton transfer between the two chambers.

Dual chamber system can advantageously avoid the reactive oxygen species generated by the electrode, which could limit or kill the bacterial cells.

An electron mediator may be provided to supply electrons to the electron transport chain via the quinone pool. The electron mediator may be an endogenous redox mediator to enhance the extracellular electron transfer rate. The electron mediator may comprise or consist of riboflavin, methyl viologen, neutral red, Anthraquinone-2,6-disulfonate and potassium ferricyanide, or combinations thereof. In a preferred embodiment, the electron mediator comprises or consists of riboflavin. The electron mediator, such as riboflavin, may be produced by the bacterial cell, or supplied externally, for example by supplementation of the media. The bacterial cell may be engineered to produce the electron mediator, such as riboflavin, methyl viologen, neutral red, anthraquinone-2,6-disulfonate or potassium ferricyanide.

In one embodiment, the electron mediator comprises flavin mononucleotide (FMN). For example, in an embodiment wherein the bacterial cell expresses MtrCAB, FMN can be added as an electron mediator, which can react with MtrC to enhance the electron transfer rate.

In another embodiment, the electron source may be from an electron donor molecule, such as an electron donor molecule capable of oxidation and associated enzyme capable of oxidising the electron donor molecule, such as a dehydrogenase. The electron source may be an organic compound, hydrogen, ammonia or an electrode, or combinations thereof. In one embodiment, the electron donor molecule may be an organic molecule. The electron donor molecule may comprise one or more of the electron donor molecules selected from formate, hydrogen, pyruvate, 2-oxogluterate, sulfur, sulfide, sulphite, thiosulphate, lactate, ethanol, glycerol, malate, succinate, gluconate, x-amines, NADH, and humics, or combinations thereof. The bacterial cell may comprise, or may be engineered to comprise, an enzyme capable of oxidising an electron donor molecule, such as a dehydrogenase or oxidase. The bacterial cell may comprise, or may be engineered to comprise, one or more of formate dehydrogenase; hydrogenase; pyruvate dehydrogenase; 2-oxoglutarat-dehydrogenase; rhodanese; sulfide dehydrogenase; sulfite oxidase; thiosulphate dehydrogenase; lactate oxidase; ethanol dehydrogenase; glycerol dehydrogenase; malate dehydrogenase; succinate dehydrogenase; gluconate dehydrogenase; amine dehydrogenase; and NADH dehydrogenase.

In one embodiment, the electron source may be a combination of two or more electron sources. For example, the electron source may be a combination of an electrode linked to a source of electricity and an electron donor molecule. The electron source may be a combination of two or more electron donor molecules. The electron source may be one or more electrodes linked to two or more sources of electricity.

### The Medium

The bacterial cell may be provided in a medium for growth and/or maintenance (i.e. culture media). The medium may be any suitable medium that is capable of growth and/or maintenance of the bacterial cell.

The medium may comprise, or be supplemented with, essential nutrients for growth or maintenance of the bacterial cell. In one embodiment, the medium comprises, or is supplemented with, the electron mediator, such as riboflavin. Additionally or alternatively, the electron mediator, such as riboflavin, may be produced by the bacterial cell or by a co-cultured cell, such as a Shewanella spp. (e.g. Shewanella oneidensis MR-1). For example, in a co-culture of Shewanella oneidensis MR-1 and engineered Ralstonia eutropha, the Shewanella oneidensis MR-1 can synthesise and secrete the electron mediator, such as riboflavin, and the Ralstonia eutropha may use the electron mediator, such as riboflavin, for electron transfer and CO₂ fixation.

In an embodiment wherein the electrons are provided by an electrode, the electrode, such as the cathode, may be submerged in the medium with the bacterial cell. Additionally or alternatively, the media may comprise, or be supplemented with, an electron donor molecule.

Additionally or alternatively, the media may comprise, or be supplemented with one or more carotenoids such as canthaxanthin, salinixanthin and echinenone. Preferably, the media comprises, or is supplemented with, canthaxanthin.

### The Rhodopsin

The bacterial cell may be engineered to express the rhodopsin. The engineering to express the rhodopsin may comprise the transformation and expression of a sequence encoding the rhodopsin. Alternatively, the expression of an endogenous rhodopsin in the bacterial cell may be enhanced, for example by the engineering of a promotor for enhanced expression of the gene encoding an endogenous rhodopsin. Alternatively, a non-functioning endogenous rhodopsin may be restored by mutation.

The rhodopsin may comprise Gloeobacter spp. rhodopsin. In one embodiment, the rhodopsin may comprise Gloeobacter violaceus rhodopsin. In another embodiment, the rhodopsin may comprise Gloeobacter violaceus PCC7421 rhodopsin.

Advantageously, Gloeobacter violaceus rhodopsin has a two-fold faster turnover rate than Proteorhodopsin (PR), and is able to bind carotenoids with a 4-keto group, e.g., salinixanthin and echinenone, to increase the absorption cross-section of the pump. Additionally, Gloeobacter violaceus rhodopsin has a high tolerance to fluctuation in pH.

The rhodopsin may comprise the sequence of

MGLMTVFSSAPELALLGSTFAQVDPSNLSVSDSLTYGQFNLVYNAFSFAI
AAMFASALFFFSAQALVGQRYRLALLVSAIVVSIAGYHYFRIFNSWDAAY
VLENGVYSLTSEKFNDAYRYVDWLLTVPLLLVETVAVLTLPAKEARPLLI
KLTVASVLMIATGYPGEISDDITTRIIWGTVSTIPFAYILYVLWVELSRS
LVRQPAAVQTLVRNMRWLLLLSWGVYPIAYLLPMLGVSGTSAAVGVQVGY
TIADVLAKPVFGLLVFAIALVKTKADQESSEPHAAIGAAANKSGGSLIS*
(Gloeobacter violaceus rhodopsin) SEQ ID NO: 13,
or a variant thereof.

The bacterial cell may be further provided with one or more carotenoids such as canthaxanthin, salinixanthin and echinenone. Preferably, the bacterial cell is further provided with canthaxanthin. The GR and carotenoid, such as canthaxanthin, may form a complex, such as a GR-canthaxanthin complex, in the bacterial cell.

Advantageously, carotenoids, such as canthaxanthin, act as an antenna of GR to improve the capture of light energy. The GR-canthaxanthin complex provides a 5-fold more proton pumping capacity compared to sole GR.

The carotenoid, such as canthaxanthin, may be provided as a supplement, such as in the culture media, or the bacterial cell may comprise the enzyme(s) necessary for biosynthesis. The bacterial cell may express, or may be engineered to express, enzyme(s) for carotenoid biosynthesis, such as canthaxanthin biosynthesis.

Canthaxanthin biosynthesis proceeds from beta-carotene via the action of beta-carotene ketolase. In one embodiment, the bacterial cell may express, or may be engineered to express beta-carotene ketolase.

### Other Cellular Components

The skilled person will recognise that in addition to a rhodopsin, and a CO₂ fixation pathway, the photoautotrophic growth required by the bacterial cell in accordance with the present invention may additionally require:
a CO₂ transporter molecule;
an ATP synthase;
a transhydrogenase;
a NADH dehydrogenase;
a quinone pool; and
an electron transport chain.

The skilled person will recognise that such components may be naturally provided in the bacterial cell. One or more, or all of these components may be engineered in the bacterial cell if necessary.

### The CO₂ Transporter Molecule

In a preferred embodiment, the bacterial cell has a native functional CO₂ transporter. CO₂ can be transported into cells as a form of CO₂ or bicarbonate (HCO3—). At least 5 transporter systems are known to the skilled person and may be provided to the cell by genetic modification: (i) BCT1, HCO3 transporter encoded by the cmpABCD (Omata et al. 1999). (ii) SbtA, HCO3—transporter (Shibata et al. 2002). (iii) BicA, HCO3 transporter (Price et al. 2004) (iv) NDH-14, CO₂ uptake system (Maeda et al. 2002; Shibata et al. 2001). (v) NDH-13, CO₂ uptake system (Klughammer et al. 1999; Maeda et al. 2002; Shibata et al. 2001) (Prommeenate et al. 2004; Zhang et al. 2004). In another embodiment, the bacterial cell may be engineered to provide a functional CO₂ transporter, for example by the transformation with a CO₂ transporter gene and/or promoter thereof.

### The ATP Synthase

In a preferred embodiment, the bacterial cell has a native functional ATP synthase. In another embodiment, the bacterial cell may be engineered to provide a functional ATP synthase, for example by the transformation with an ATP synthase gene and/or promoter thereof.

### The Transhydrogenase

In a preferred embodiment, the bacterial cell has a native functional transhydrogenase. In another embodiment, the bacterial cell may be engineered to provide a functional transhydrogenase, for example by the transformation with a transhydrogenase gene and/or promoter thereof.

### The NADH Dehydrogenase

In a preferred embodiment, the bacterial cell has a native functional NADH dehydrogenase. In another embodiment, the bacterial cell may be engineered to provide a functional NADH dehydrogenase, for example by the transformation with a NADH dehydrogenase gene and/or promoter thereof.

### The Quinone Pool

The quinone pool may be an endogenous quinone pool in the membrane of the bacterial cell. The quinone may be ubiqunone.

### The Electron Transport Chain

In a preferred embodiment, the bacterial cell has a native functional electron transport chain. The electron transport chain may be an endogenous electron transport chain in the membrane of the bacterial cell. In another embodiment, the bacterial cell may be engineered to provide a functional electron transport chain, for example by the transformation with one or more, or all, of the genes required for a functional electron transport chain.

In one embodiment, the bacterial cell expresses, or is engineered to express MtrCAB.

MtrCAB is a multi-heme protein complex linking the intracellular electron transport chain with extracellular substrates. Advantageously, the electron transport proteins encoded in MtrCAB genes enables cells to take electrons directly from an electrode.

Advantageously, the energy transfer efficiency can be increased from 20% to 45% by expression of MtrCAB and carbonic anhydrase (can), and addition of carotenoids, such as canthaxanthin.

The MtrCAB may be recombinant and/heterogenous. The MtrCAB may be heterogeneously expressed. The MtrCAB may be derived from a Shewanella spp. such as Shewanella oneidensis MR-1.

In one embodiment, the bacterial cell is transformed with nucleic acid, such as plasmid DNA, encoding the MtrCAB gene cluster. The plasmid may comprise a selection marker. The MtrCAB may be expressed under the control of a suitable promoter, such as the P_{BAD} promoter. The promoter may be inducible or repressible. The MtrCAB gene cluster may be maintained extrachromosomally or stably integrated into the chromosomal DNA.

MtrA may comprise or consist of the sequence
of SEQ ID NO: 15
(MKNCLKMKNLLPALTITMAMSAVMALVVTPNAYASKWDEKMTPEQVEATL
DKKFAEGNYSPKGADSCLMCHKKSEKVMDLFKGVHGAIDSSKSPMAGLQC
EACHGPLGQHNKGGNEPMITFGKQSTLSADKQNSVCMSCHQDDKRMSWNG
GHHDNADVACASCHQVHVAKDPVLSKNTEMEVCTSCHTKQKADMNKRSSH
PLKWAQMTCSDCHNPHGSMTDSDLNKPSVNDTCYSCHAEKRGPKLWEHAP
VTENCVTCHNPHGSVNDGMLKTRAPQLCQQCHASDGHASNAYLGNTGLGS
NVGDNAFTGGRSCLNCHSQVHGSNHPSGKLLQR).
MtrB may comprise or consist of the sequence
of SEQ ID NO: 16
(MKFKLNLITLALLANTGLAVAADGYGLANANTEKVKLSAWSCKGCVVETG
TSGTVGVGVGYNSEEDIRSANAFGTSNEVAGKFDADLNFKGEKGYRASVD
AYQLGMDGGRLDVNAGKQGQYNVNVNYRQIATYDSNSALSPYAGIGGNNL
TLPDNWITAGSSNQMPLLMDSLNALELSLKRERTGLGFEYQGESLWSTYV
NYMREEKTGLKQASGSFFNQSMMLAEPVDYTTDTIEAGVKLKGDRWFTAL
SYNGSIFKNEYNQLDFENAFNPTFGAQTQGTMALDPDNQSHTVSLMGQYN
DGSNALSGRILTGQMSQDQALVTDNYRYANQLNTDAVDAKVDLLGMNLKV
VSKVSNDLRLTGSYDYYDRDNNTQVEEWTQISINNVNGKVAYNTPYDNRT
QRFKVAADYRITRDIKLDGGYDFKRDQRDYQDRETTDENTVWARLRVNSF
DTWDMWVKGSYGNRDGSQYQASEWTSSETNSLLRKYNLADRDRTQVEARI
THSPLESLTIDVGARYALDDYTDTVIGLTESKDTSYDANISYMITADLLA
TAFYNYQTIESEQAGSSNYSTPTWTGFIEDQVDVVGAGISYNNLLENKLR
LGLDYTYSNSDSNTQVRQGITGDYGDYFAKVHNINLYAQYQATEKLALRF
DYKIENYKDNDAANDIAVDGIWNVVGFGSNSHDYTAQMLMLSMSYKL).
MtrC may comprise or consist of the sequence
of SEQ ID NO: 17
(MMNAQKSKIALLLAASAVTMALTGCGGSDGNNGNDGSDGGEPAGSIQTLN
LDITKVSYENGAPMVTVFATNEADMPVIGLANLEIKKALQLIPEGATGPG
NSANWQGLGSSKSYVDNKNGSYTFKFDAFDSNKVFNAQLTQRFNVVSAAG
KLADGTTVPVAEMVEDFDGQGNAPQYTKNIVSHEVCASCHVEGEKIYHQA
TEVETCISCHTQEFADGRGKPHVAFSHLIHNVHNANKAWGKDNKIPTVAQ
NIVQDNCQVCHVESDMLTEAKNWSRIPTMEVCSSCHVDIDFAAGKGHSQQ
LDNSNCIACHNSDWTAELHTAKTTATKNLINQYGIETTSTINTETKAATI
SVQVVDANGTAVDLKTILPKVQRLEIITNVGPNNATLGYSGKDSIFAIKN
GALDPKATINDAGKLVYTTTKDLKLGQNGADSDTAFSFVGWSMCSSEGKF
VDCADPAFDGVDVTKYTGMKADLAFATLSGKAPSTRHVDSVNMTACANCH
TAEFEIHKGKQHAGFVMTEQLSHTQDANGKAIVGLDACVTCHTPDGTYSF
ANRGALELKLHKKHVEDAYGLIGGNCASCHSDFNLESFKKKGALNTAAAA
DKTGLYSTPITATCTTCHTVGSQYMVHTKETLESFGAVVDGTKDDATSAA
QSETCFYCHTPTVADHTKVKM).
The MtrCAB gene cluster may comprise the sequence
of SEQ ID NO: 34
(ATGATGAACGCACAAAAATCAAAAATCGCACTGCTGCTCGCAGCAAGTGC
CGTCACAATGGCCTTAACCGGCTGTGGTGGAAGCGATGGTAATAACGGCA
ATGATGGTAGTGATGGTGGTGAGCCAGCAGGTAGCATCCAGACGTTAAAC
CTAGATATCACTAAAGTAAGCTATGAAAATGGTGCACCTATGGTCACTGT
TTTCGCCACTAACGAAGCCGACATGCCAGTGATTGGTCTCGCAAATTTAG
AAATCAAAAAAGCACTGCAATTAATACCGGAAGGGGCGACAGGCCCAGGT
AATAGCGCTAACTGGCAAGGCTTAGGCTCATCAAAGAGCTATGTCGATAA
TAAAAACGGTAGCTATACCTTTAAATTCGACGCCTTCGATAGTAATAAGG
TCTTTAATGCTCAATTAACGCAACGCTTTAACGTTGTTTCTGCTGCGGGT
AAATTAGCAGACGGAACGACCGTTCCCGTTGCCGAAATGGTTGAAGATTT
CGACGGCCAAGGTAATGCGCCGCAATATACAAAAAATATCGTTAGCCACG
AAGTATGTGCTTCTTGCCACGTAGAAGGTGAAAAGATTTATCACCAAGCT
ACTGAAGTCGAAACTTGTATTTCTTGCCACACTCAAGAGTTTGCGGATGG
TCGCGGCAAACCCCATGTCGCCTTTAGTCACTTAATTCACAATGTGCATA
ATGCCAACAAAGCTTGGGGCAAAGACAATAAAATCCCTACAGTTGCACAA
AATATTGTCCAAGATAATTGCCAAGTTTGTCACGTTGAATCCGACATGCT
CACCGAGGCAAAAAACTGGTCACGTATTCCAACAATGGAAGTCTGTTCTA
GCTGTCACGTAGACATCGATTTTGCTGCGGGTAAAGGCCACTCTCAACAA
CTCGATAACTCCAACTGTATCGCCTGCCATAACAGCGACTGGACTGCTGA
GTTACACACAGCCAAAACCACCGCAACTAAGAACTTGATTAATCAATACG
GTATCGAGACTACCTCGACAATTAATACCGAAACTAAAGCAGCCACAATT
AGTGTTCAAGTTGTAGATGCGAACGGTACTGCTGTTGATCTCAAGACCAT
CCTGCCTAAAGTGCAACGCTTAGAGATCATCACCAACGTTGGTCCTAATA
ATGCAACCTTAGGTTATAGTGGCAAAGATTCAATATTTGCAATCAAAAAT
GGAGCTCTTGATCCAAAAGCTACTATCAATGATGCTGGCAAACTGGTTTA
TACCACTACTAAAGACCTCAAACTTGGCCAAAACGGCGCAGACAGCGACA
CAGCATTTAGCTTTGTAGGTTGGTCAATGTGTTCTAGCGAAGGTAAGTTT
GTAGACTGTGCAGACCCTGCATTTGATGGTGTTGATGTAACTAAGTATAC
CGGCATGAAAGCGGATTTAGCCTTTGCTACTTTGTCAGGTAAAGCACCAA
GTACTCGCCACGTTGATTCTGTTAACATGACAGCCTGTGCCAATTGCCAC
ACTGCTGAGTTCGAAATTCACAAAGGCAAACAACATGCAGGCTTTGTGAT
GACAGAGCAACTATCACACACCCAAGATGCTAACGGTAAAGCGATTGTAG
GCCTTGACGCATGTGTGACTTGTCATACTCCTGATGGCACCTATAGCTTT
GCCAACCGTGGTGCGCTAGAGCTAAAACTACACAAAAAACACGTTGAAGA
TGCCTACGGCCTCATTGGTGGCAATTGTGCCTCTTGTCACTCAGACTTCA
ACCTTGAGTCTTTCAAGAAGAAAGGCGCATTGAATACTGCCGCTGCAGCA
GATAAAACAGGTCTATATTCTACGCCGATCACTGCAACTTGTACTACCTG
TCACACAGTTGGCAGCCAGTACATGGTCCATACGAAAGAAACCCTGGAGT
CTTTCGGTGCAGTTGTTGATGGCACAAAAGATGATGCTACCAGTGCGGCA
CAGTCAGAAACCTGTTTCTACTGCCATACCCCAACAGTTGCAGATCACAC
TAAAGTGAAAATGTAATTTGCCCAAGCAGGGGGAGCTCGCTCCCCCTTTC
TTGAATTTTGTTGGGACAAATTGGGAAGCCTATTATGAAGAACTGCCTAA
AAATGAAAAACCTACTGCCGGCACTTACCATCACAATGGCAATGTCTGCA
GTTATGGCATTAGTCGTCACACCAAACGCTTATGCGTCGAAGTGGGATGA
GAAAATGACGCCAGAGCAAGTCGAAGCCACCTTAGATAAGAAGTTTGCCG
AAGGCAACTACTCCCCTAAAGGCGCCGATTCTTGCTTGATGTGCCATAAG
AAATCCGAAAAAGTCATGGACCTTTTCAAAGGTGTCCACGGTGCGATTGA
CTCCTCTAAGAGTCCAATGGCTGGCCTGCAATGTGAGGCATGCCACGGCC
CACTGGGTCAGCACAACAAAGGCGGCAACGAGCCGATGATCACTTTTGGT
AAGCAATCAACCTTAAGTGCCGACAAGCAAAACAGCGTATGTATGAGCTG
TCACCAAGACGATAAGCGTATGTCTTGGAATGGCGGTCACCATGACAATG
CCGATGTTGCTTGTGCTTCTTGTCACCAAGTACACGTCGCAAAAGATCCT
GTGTTATCTAAAAACACGGAAATGGAAGTCTGTACTAGCTGCCATACAAA
GCAAAAAGCGGATATGAATAAACGCTCAAGTCACCCACTCAAATGGGCAC
AAATGACCTGTAGCGACTGTCACAATCCCCATGGGAGCATGACAGATTCC
GATCTTAACAAGCCTAGCGTGAATGATACCTGTTATTCCTGTCACGCCGA
AAAACGCGGCCCAAAACTTTGGGAGCATGCACCCGTCACTGAGAATTGTG
TCACTTGCCACAATCCTCACGGTAGTGTGAATGACGGTATGCTGAAAACC
CGTGCGCCACAGCTATGTCAGCAATGTCACGCCAGCGATGGCCACGCCAG
CAACGCCTACTTAGGTAACACTGGATTAGGTTCAAATGTCGGTGACAATG
CCTTTACTGGTGGAAGAAGCTGCTTAAATTGCCATAGTCAGGTTCATGGT
TCTAACCATCCATCTGGCAAGCTATTACAGCGCTAAGGAGACGAGAAAAT
GAAATTTAAACTCAATTTGATCACTCTAGCGTTATTAGCCAACACAGGCT
TGGCCGTCGCTGCTGATGGTTATGGTCTAGCGAATGCCAATACTGAAAAA
GTGAAATTATCCGCATGGAGCTGTAAAGGCTGCGTCGTTGAAACGGGCAC
ATCAGGCACTGTGGGTGTCGGTGTCGGTTATAACAGCGAAGAGGATATTC
GCTCTGCCAATGCCTTTGGTACATCCAATGAAGTGGCGGGTAAATTTGAT
GCCGATTTAAACTTTAAAGGTGAAAAGGGTTATCGTGCCAGTGTTGATGC
TTATCAACTCGGTATGGATGGCGGTCGCTTAGATGTCAATGCGGGCAAAC
AAGGCCAGTACAACGTCAATGTGAACTATCGCCAAATTGCTACCTACGAC
AGCAATAGCGCCCTATCGCCCTACGCGGGTATTGGTGGCAATAACCTCAC
GTTACCGGATAACTGGATAACAGCAGGTTCAAGCAACCAAATGCCACTCT
TGATGGACAGCCTCAATGCCCTCGAACTCTCACTTAAACGTGAGCGCACG
GGGTTGGGATTTGAATATCAAGGTGAATCCCTGTGGAGCACCTATGTTAA
CTACATGCGTGAAGAGAAAACCGGCTTAAAACAAGCCTCTGGTAGCTTCT
TCAACCAATCGATGATGTTAGCAGAGCCGGTGGATTACACCACTGACACC
ATTGAAGCGGGTGTCAAACTCAAGGGTGATCGTTGGTTTACCGCACTCAG
TTACAATGGGTCAATATTCAAAAACGAATACAACCAATTGGACTTTGAAA
ATGCTTTTAACCCCACCTTTGGTGCTCAAACCCAAGGTACGATGGCACTC
GATCCGGATAACCAGTCACACACCGTGTCGCTGATGGGACAGTACAACGA
TGGCAGCAACGCACTGTCGGGTCGTATTCTGACCGGACAAATGAGCCAAG
ATCAGGCGTTAGTGACGGATAACTACCGTTATGCTAATCAGCTCAATACC
GATGCCGTCGATGCCAAAGTCGATCTACTGGGTATGAACCTGAAAGTCGT
TAGCAAAGTGAGCAATGATCTTCGCTTAACAGGTAGTTACGATTATTACG
ACCGTGACAATAATACCCAAGTAGAAGAATGGACTCAGATCAGCATCAAC
AATGTCAACGGTAAGGTGGCTTATAACACCCCTTACGATAATCGTACGCA
ACGCTTTAAAGTTGCCGCAGATTATCGCATTACCCGCGATATCAAACTCG
ATGGTGGTTATGACTTCAAACGTGACCAACGTGATTATCAAGACCGTGAA
ACCACGGATGAAAATACCGTTTGGGCCCGTTTACGTGTAAACAGCTTCGA
TACTTGGGACATGTGGGTAAAAGGCAGTTACGGTAACCGTGACGGCTCAC
AATACCAAGCGTCTGAATGGACCTCTTCTGAAACCAACAGCCTGTTACGT
AAGTACAATCTGGCTGACCGTGACAGAACTCAAGTCGAAGCACGGATCAC
CCATTCGCCATTAGAAAGCCTGACTATCGATGTTGGTGCCCGTTACGCGT
TAGATGATTATACCGATACTGTGATTGGATTAACTGAGTCAAAAGACACC
AGTTATGATGCCAACATCAGTTATATGATCACCGCTGACTTACTGGCAAC
CGCCTTCTACAATTACCAAACCATTGAGTCTGAACAGGCGGGTAGCAGCA
ATTACAGCACCCCAACGTGGACAGGCTTTATAGAAGATCAGGTAGATGTG
GTCGGTGCAGGTATCAGCTACAACAATCTGCTGGAGAACAAGTTACGCCT
AGGACTGGACTACACCTATTCCAACTCCGACAGTAACACTCAAGTCAGAC
AAGGTATCACTGGCGACTATGGTGATTATTTTGCCAAAGTGCATAACATT
AACTTATACGCTCAATATCAAGCCACCGAGAAACTCGCGCTGCGCTTCGA
TTACAAAATTGAGAACTATAAGGACAATGACGCCGCAAATGATATCGCCG
TTGATGGCATTTGGAACGTCGTAGGTTTTGGTAGTAACAGCCATGACTAC
ACCGCACAAATGCTGATGCTGAGCATGAGTTACAAACTCTAA)

The bacterial cell may be further modified to produce cytochromes aerobically, such as cytochrome c, for example for heme insertion. Aerobic cytochrome production, such as the production of cytochrome c, may be provided by replacing the cytochrome promoter with an alternative promoter, which functions under aerobic conditions, such as a constitutive promoter.

In one embodiment, the electron transport chain may comprise a nitrate reductase, e.g. as an electron acceptor molecule. In one embodiment, the bacterial cell expresses, or is engineered to express nitrate reductase. The nitrate reductase may be endogenous to the bacterial cell or heterogenous.

### The CO₂ Fixation Pathway

The skilled person will recognise that any CO₂ fixation pathway may be provided, which may be naturally occurring or engineered. In a preferred embodiment the bacterial cell possesses the enzymes of the Calvin cycle.

For example Fuch (Annu Rev Microbiol. 2011; 65:631-58. doi: 10.1146/annurev-micro-090110-102801; https://pubmed.ncbi.nlm.nih.gov/21740227/) (incorporated herein by reference) describes five examples of alternative CO₂ fixation pathways, which may be provided. Alternatively, a designed/engineered CO₂ fixation pathway may be provided, for example as described by Schwander et al. (Science. 2016 Nov. 18; 354 (6314): 900-904: https://www.ncbi.nlm.nih.gov/pmc/articles/PMC5892708/) (incorporated herein by reference).

In a preferred embodiment, the bacterial cell has a native functional Calvin cycle for CO₂ fixation. In another embodiment, the bacterial cell may be engineered to provide a functional Calvin cycle, for example by the transformation with one or more, or all of the genes required for a functional Calvin cycle.

In one embodiment, the bacterial cell expresses, or is engineered to express one or more of the Calvin cycle enzymes selected from RuBisCO, FBPase, SBPase, PRK, PGK, GADPH, TPI, Fructose-1,6-bisphosphate aldolase, Transketolase, RPE, and Ribose-5-phosphate isomerase. In one embodiment, the bacterial cell expresses, or is engineered to express all of the Calvin cycle enzymes selected from RuBisCO, FBPase, SBPase, PRK, PGK, GADPH, TPI, Fructose-1,6-bisphosphate aldolase, Transketolase, RPE, and Ribose-5-phosphate isomerase.

In one embodiment, the bacterial cell expresses, or is engineered to express a carbonic anhydrase (can). In one embodiment, the bacterial cell is engineered to overexpress a carbonic anhydrase. Preferably an endogenous carbonic anhydrase is overexpressed.

Advantageously, the expression of carbonic anhydrase can improve the system by concentrating CO₂ and it is favourable to CO₂ fixation. Abundant carbonic anhydrase can increase the CO₂ utilisation rate.

In one embodiment, the bacterial cell is transformed with nucleic acid, such as plasmid DNA, encoding the carbonic anhydrase. The plasmid may comprise a selection marker. The carbonic anhydrase may be expressed under the control of a suitable promoter, such as the P_{BAD} promoter. The promoter may be inducible or repressible. The carbonic anhydrase gene may be maintained extrachromosomally or stably integrated into the chromosomal DNA.

The carbonic anhydrase may be the carbonic anhydrase (can) of Ralstonia eutropha. The carbonic anhydrase may comprise the sequence of SEQ ID NO: 18 (MTDAIAQLFRNNREWVDRVNAEDPTFFMRLANQQAPEYLWIGCSDSRVPANQI LGLAPGEVFVHRNIANVIAHSDLNALAVIQFAVEVLKVRHITVVGHYGCGGVKV ALKRERIGLADNWLRHVRDVADKHEAYLGTLLREDDAHTRLCELNVIEQVNNV CQTTVLQDAWSRGQAVTVHGWVYGVSDGLLRDLGMAASSNDELREQLAAAYR QYGDPPQASIR).

### Organic Product Production and Other Functions

The bacterial cells may be capable of producing organic product, such as biomass through growth of the cells and/or organic molecules via the CO₂ fixation pathway, such as the Calvin cycle.

The bacterial cells may be further engineered for the production of an organic product. For example, the bacterial cells may be engineered to synthesise an enzyme for conversion of a Calvin cycle product, or downstream product thereof, to an alternative organic product.

The organic products may comprise products selected from sugars, carbohydrates, peptides, polypeptides, nucleic acids, small molecules, drugs, pro-drugs, Polyhydroxybutyrate (PHB), riboflavin (Vitamin B2), alcohols (e.g. ethanol), carotenoids and alkane; or combinations thereof.

The bacterial cells may be engineered for any suitable function requiring biosynthesis, conversion or degradation of products. The bacterial cells may be engineered for the provision of energy, which may for example drive any biosynthesis or biodegradation pathways in cells.

In one embodiment, the bacterial cell is transformed with nucleic acid encoding a product for expression. The product may comprise a polypeptide, such as a peptide or a protein. In another embodiment, the product may comprise a nucleic acid, such as RNA or DNA. The RNA may comprise mRNA, miRNA, siRNA, tRNA or rRNA. The polypeptide may be a biologically active agent (e.g. a biological therapeutic molecule). The polypeptide may be an enzyme. The polypeptide may be a drug, or pro-drug. Both polypeptides and nucleic acid products may be encoded. The product for expression may comprise an antibody, or antibody fragment, or mimetic thereof. The product for expression may comprise an immunogenic peptide or polypeptide, such as a vaccine for mammals. The product for expression may comprise a biological drug, such as a biological drug for cancer therapy or prevention. The product for expression may comprise insulin, for example for diabetes therapy.

The product for expression may comprise an enzyme catalyst that is capable of producing a biochemical compound, such as a therapeutic drug. A plurality of enzyme catalysts may be provided for expression such that a multi-step reaction can be provided to produce a biochemical compound. The therapeutic drug may comprise a cytotoxic drug, such as catechol. The product for expression may comprise SalA and/or SalR. The skilled person will recognise that when salicylic acid (Aspirin) is present it combines with SalR to yield an active form SalR*, which then initiates transcription of salA and salR (positive feedback). Then SalA or salicylate hydroxylase converts salicylic acid to catechol in the presence of NADH. Additionally or alternatively, salicylate hydroxylase (salA) may be provided to produce chlorocatechol or hydroxyanthranilate.

In one embodiment, the bacterial cell is transformed with nucleic acid encoding a product for replication, such as cloning. For example, the bacterial cells may be used for the production of plasmids, or viral nucleic acid, such as viral vectors. For example, the bacterial cells may be used for the production of DNA-vaccines or nucleic acid for gene therapy. In one embodiment, the bacterial cell is transformed with nucleic acid encoding a virus particle (or parts thereof) and accompanying viral nucleic acid (or parts thereof). The virus may be a virus that is a eukaryote-based virus, such as a mammalian virus. The virus may be an attenuated or non-replicating virus. In one embodiment the product for expression may comprise a HPV vaccine, such as HPV polypeptides and/or nucleic acid encoding HPV genes. In another embodiment, the product for expression may comprise a phage, or parts thereof.

The product for expression may comprise a membrane polypeptide (i.e. a polypeptide comprising a hydrophobic domain arranged to be anchored in a membrane bilayer).

The expression of the product for expression may be regulated. For example, the expression of the product for expression may be under the control of an inducible or repressible promoter. For example, the expression of the product for expression may under the control of the MphR regulation system that is inducible by erythromycin. In another embodiment, the expression of the product for expression may be constitutive. In particular, a constitutive promoter may be encoded for promoting the expression of the product for expression. In one embodiment, the expression of the product for expression may be controlled by a strong promoter, such as a viral promoter. The promoter may comprise CMV promoter, SV40.

### The Light Source

In a preferred embodiment, the light source is natural light, such as sunlight. Additionally or alternatively, artificial light may be used.

### Other Aspects

According to another aspect of the invention there is provided a bacterial cell that is recombinantly engineered for photoautotrophic CO₂ fixation, the bacterial cell comprising:
a rhodopsin and the components for a CO₂ fixation pathway for biosynthesis of organic molecules from CO₂.

The bacterial cell may be engineered for photoautotrophic CO₂ fixation in the presence of an electron source for donation of electrons into the electron transport chain via the quinone pool. The electron source may be as described herein.

According to another aspect of the invention there is provided the use of the recombinantly engineered bacterial cell according to the invention, or the electromicrobial system of the invention, for CO₂ fixation and/or biosynthesis of organic molecules.

According to another aspect of the invention there is provided a method of CO₂ fixation and/or biosynthesis of organic product, the method comprising:
providing the electromicrobial system of the invention, and
culturing the bacterial cells in the presence of light and CO₂.

Preferably the light is sunlight. The CO₂ may be atmospheric CO₂.

The method may further comprise sub-culturing the bacterial cells for maintenance and/or growth of the culture.

The method may further comprise the harvesting of the organic products, for example by purification or isolation of the product from the culture media and/or cells.

The organic product may be biomass. The organic product may be an organic molecule. The organic product may be any one or more of the organic molecules selected from Polyhydroxybutyrate (PHB), riboflavin (Vitamin B2), alcohols (e.g. ethanol), carotenoids and alkane.

According to another aspect of the invention there is provided a nucleic acid encoding one or more, or all, genes of the crt operon genes, and further encoding the genes of blh and/or rhodopsin genes (such as Gloeobacter violaceus rhodopsin (GR)).

The nucleic acid may further encode the dxr gene.

The nucleic acid may be DNA. The nucleic acid may be a plasmid.

The nucleic acid may comprise the sequence of

SEQ ID NO: 14
ACATGGTACTCCGTCAAGCCGTCAATTGTCTGATTCGTTACCAATTATGACAACTTGACGGCTACA
TCATTCACTTTTTCTTCACAACCGGCACGGAACTCGCTCGGGCTGGCCCCGGTGCATTTTTTAAA
TACCCGCGAGAAATAGAGTTGATCGTCAAAACCAACATTGCGACCGACGGTGGCGATAGGCATCC
GGGTGGTGCTCAAAAGCAGCTTCGCCTGGCTGATACGTTGGTCCTCGCGCCAGCTTAAGACGCTA
ATCCCTAACTGCTGGCGGAAAAGATGTGACAGACGCGACGGCGACAAGCAAACATGCTGTGCGAC
GCTGGCGATATCAAAATTGCTGTCTGCCAGGTGATCGCTGATGTACTGACAAGCCTCGCGTACCC
GATTATCCATCGGTGGATGGAGCGACTCGTTAATCGCTTCCATGCGCCGCAGTAACAATTGCTCA
AGCAGATTTATCGCCAGCAGCTCCGAATAGCGCCCTTCCCCTTGCCCGGCGTTAATGATTTGCCC
AAACAGGTCGCTGAAATGCGGCTGGTGCGCTTCATCCGGGCGAAAGAACCCCGTATTGGCAAATA
TTGACGGCCAGTTAAGCCATTCATGCCAGTAGGCGCGCGGACGAAAGTAAACCCACTGGTGATAC
CATTCGCGAGCCTCCGGATGACGACCGTAGTGATGAATCTCTCCTGGCGGGAACAGCAAAATATC
ACCCGGTCGGCAAACAAATTCTCGTCCCTGATTTTTCACCACCCCCTGACCGCGAATGGTGAGAT
TGAGAATATAACCTTTCATTCCCAGCGGTCGGTCGATAAAAAAATCGAGATAACCGTTGGCCTCA
ATCGGCGTTAAACCCGCCACCAGATGGGCATTAAACGAGTATCCCGGCAGCAGGGGATCATTTTG
CGCTTCAGCCATACTTTTCATACTCCCGCCATTCAGAGAAGAAACCAATTGTCCATATTGCATCA
GACATTGCCGTCACTGCGTCTTTTACTGGCTCTTCTCGCTAACCAAACCGGTAACCCCGCTTATT
AAAAGCATTCTGTAACAAAGCGGGACCAAAGCCATGACAAAAACGCGTAACAAAAGTGTCTATAA
TCACGGCAGAAAAGTCCACATTGATTATTTGCACGGCGTCACACTTTGCTATGCCATAGCATTTT
TATCCATAAGATTAGCGGATCCTACCTGACGCTTTTTATCGCAACTCTCTACTGTTTCTCCATAC
CCGTTTTTTTGGGCTAGCTAAGGAGGAGACCCCATGGGAGAGCTCATGCATCGCATTACCATCCT
GGGCGCCACCGGCTCCATTGGCGAAAGCACGCTCGACGTCGTCAGGCGCCATGCCGACCGCTATG
TGGTGCATGCGCTGACCGCCCACCGGCAGGTGCGCAAGCTGGCTGACCAGTGCGTTGAGTTCCGC
CCGGCCCGCGCCGTGGTGGGGACCGCCGAGGCAGCGCTCGAACTGGAAACCCTGCTGCGCGATGC
CGGCGTGAAGACCGAGGTCAGCCACGGCGAAGCCGCGCTGGAATCCGTGGCCGCCGATGCGCAGA
CCGATTCGGTGATGGCCGCCATCGTCGGCGCCGCCGGCCTGCGCCCGACGCTGGCGGCAGCACGT
GCCGGCAAGCGCGTGCTGCTGGCCAACAAGGAAGCGCTGGTGATGTCCGGgcGCATCTTCATGGA
TGCGGTGCGCGAGCACGGCGCCACCTTGCTGCCGATCGACAGCGAGCACAACGCCATCTTCCAGT
GCCTTCCGGCAGATGATCCGCGTTATGGTCGTGgtGTTGCAAGGGTTCTGTtGACCGCAtcAGGA
GGtccGTTccgTACGCGTGACCCGGCAACGCTGCACGATATCtcCCCCGACCAGGCATGCGCACA
TCCCAACTGGGTTATGGGTCGCAAGATCTCGGTCGATTCCGCCACCATGATGAACAAGGGCCTCG
AAGTCATCGAGGCGCACTGGCTGTTCGGCGCCCCCGCCGAGCGCATCGAGGTACTGATCCATCCG
CAGAGCATCGTGCATTCGATGGTGGCCTACACCGACGGCTCGGTGCTGGCGCAACTGGGCAACCC
GGACATGCGCACGCCGATCGCCTATGGCCTGGCGTATCCGGAGCGGATCGATGCGGGGGTCACGC
CGCTGGACCTGACCGTGGCTGGGGGCTTGCATTTCGAGAAGCCGGACCTGGTGCGCTTCCCGTGC
CTGGGCCTGGCCTTCGATGCACTGCGCGCCGGCGGTGTGGCGCCGGCGGCCCTCAATGCAGCCAA
CGAGGTGGCGGTCGAGGCATTCCTGGGCGGTACAGTGCGCTTCACGGACATTGCCGGTATCGTCC
GGCAGGTGCTTGAGGCGACACCCCAGGGGCCTGCCGATACGCTCGAAGCGGTGCTGTCAGCCGAC
GCGCTGGCGCGCGAGGCGGCACGCGAGGGCGTGGCCGCCCTGGCTGCAAAGCGCTGAAGCTTAAG
GAGGAGACCCCATGGGCGGCCTGATGCTGATCGACTGGTGCGCCCTGGCCCTGGTGGTCTTTATT
GGTCTCCCGCACGGGGCACTGGACGCGGCCATCTCGTTCTCGATGATCTCGTCGGCCAAGCGCAT
CGCCCGCCTGGCCGGCATCCTGCTGATCTACCTGCTGCTGGCCACCGCCTTCTTCCTGATCTGGT
ACCAGCTGCCGGCCTTCTCGCTGCTGATCTTCCTGCTGATCTCGATCATCCACTTCGGCATGGCC
GACTTCAACGCCTCGCCGTCGAAGCTGAAGTGGCCGCACATCATCGCCCACGGCGGCGTGGTGAC
CGTGTGGCTGCCGCTGATCCAGAAGAACGAAGTGACCAAGCTGTTCTCGATCCTGACCAACGGCC
CGACCCCGATCCTGTGGGACATCCTGCTGATCTTCTTCCTGTGCTGGTCGATCGGCGTGTGCCTG
CACACCTACGAAACCCTGCGCTCGAAGCACTACAACATCGCCTTCGAACTGATCGGCCTGATCTT
CCTGGCCTGGTACGCCCCGCCGCTGGTGACCTTCGCCACCTACTTCTGCTTCATCCACTCGCGCC
GCCACTTCTCGTTCGTGTGGAAGCAGCTGCAGCACATGTCGTCGAAGAAGATGATGATCGGCTCG
GCCATCATCCTGTCGTGCACCTCGTGGCTGATCGGCGGCGGCATCTACTTCTTCCTGAACTCGAA
GATGATCGCCTCGGAAGCCGCCCTGCAGACCGTGTTCATCGGCCTGGCCGCCCTGACCGTGCCGC
ACATGATCCTGATCGACTTCATCTTCCGCCCGCACTCGTCGCGCATCAAGATCAAGAACAAGGGC
GAACTGGAAGGCAAGCCGATCCCGAACCCGCTGCTGGGCCTGGACTCGACCCGCACCGGCCATCA
TCACCATCACCATTAAGAGCTCGaTcTCAAaCaGgaTtGGgCATAAAACaGTtGAAAGTGAATAA
AGTTGcTAAGCTggTAACTCAGATCcTAAAAACaGGGATAAGACTGTGCAACGTTATGGATTGAT
GGCGCTTTTGcTCGTTTCCTGCTGGGCCAGCGCGCATAACATCGTCATCGGGCAGCCCcTTCCGT
CGGTTTTTATTGCGGATAAAGGTGAAATGCGGCTGGATGGCGGCAAGGTTAACTATCAAAAAtgG
AACAGCCTGTCTCTTCCGGgTCGGACACGTTTAGTTATTCATGTTGCAGGACGATTGTCGGCCAA
AGAGCAGTCCGCCCCGCTTATTGCGGCCCTGCAGCGCGCCAACCTGCCACAAGACCGGTTCCAGA
CCACAACCATCGTGAATACAGATGATGCTTTGCCTGGCAGCAGTCTGTTTGTGATTAACAGTATC
CGCTCCAGTAAAAAAGCCTCACCATGGCAACAATTTATTATCGACAGTAGCGGCGTGGCACAACA
TCGCTGGCAGCTTAAGCCAGAAGGTGCCGCTGTCATCGTGCTGGACCCTGATGGTCAGGTAAAGT
TTGCGAAAGACACGGCGCTCAGTGCGGATGATGTTTCTCAGGTCATTGCAACATTGCGTGCGCTG
GCAGGCTGATCCTGGCAACCCGGTAAAGGTACCGCACGGTCTGCCAATCCGACGGAGGTTTATGA
ATTTTCCACCTTTTCCACAAGCTCAACTAGTATTAACGATGTGGATTTAGCAAAAAAAACCTGTA
ACCCTAAATGTAAAATAACGGGTAAGCCTGCCAACCATGTTATGGCAGATTAAGCGTCTTTTTGA
AGGGCACCGCATCTTTCGCGTTGCCGTAAATGTATCCGTTTATAAGGACAGCCCGAATGACGGTC
TGCGCAAAAAAACACGTTCATCTCACTCGCGATGCTGCGGAGCAGTTACTGGCTGATATTGATCG
ACGCCTTGATCAGTTATTGCCCGTGGAGGGAGAACGGGATGTTGTGGGTGCCGCGATGCGTGAAG
GTGCGCTGGCACCGGGAAAACGTATTCGCCCCATGTTGCTGTTGCTGACCGCCCGCGATCTGGGT
TGCGCTGTCAGCCATGACGGATTACTGGATTTGGCCTGTGCGGTGGAAATGGTCCACGCGGCTTC
GCTGATCCTTGACGATATGCCCTGCATGGACGATGCGAAGCTGCGGCGCGGACGCCCTACCATTC
ATTCTCATTACGGAGAGCATGTGGCAATACTGGCGGCGGTTGCCTTGCTGAGTAAAGCCTTTGGC
GTAATTGCCGATGCAGATGGCCTCACGCCGCTGGCAAAAAATCGGGCGGTTTCTGAACTGTCAAA
CGCCATCGGCATGCAAGGATTGGTTCAGGGTCAGTTCAAGGATCTGTCTGAAGGGGATAAGCCGC
GCAGCGCTGAAGCTATTTTGATGACGAATCACTTTAAAACCAGCACGCTGTTTTGTGCCTCCATG
CAGATGGCCTCGATTGTTGCGAATGCCTCCAGCGAAGCGCGTGATTGCCTGCATCGTTTTTCACT
TGATCTTGGTCAGGCATTTCAACTGCTGGACGATTTGACCGATGGCATGACCGACACCGGTAAGG
ATAGCAATCAGGACGCCGGTAAAATCGACGCTGGTCAATCTGTTAGGCCCGAGGGCGGTTGAAGA
ACGTCTGAGACAACATCTTCAGCTTGCCAGTGAGCATCTCTCTGCGGCCTGCCAACACGGGCACG
CCACTCAACATTTTATTCAGGCCTGGTTTGACAAAAAACTCGCTGCCGTCAGTTAAGGATGCTGC
ATGAGCCATTTCGCGGCGATCGCACCGCCTTTTTACAGCCATGTTCGCGCATTACAGAATCTCGC
TCAGGAACTGGTCGCGCGCGGTCATCGGGTGACCTTTATTCAGCAATACGATATTAAACACTTGA
TCGATAGCGAAACCATTGGATTTCATTCCGTCGGGACAGACAGCCATCCCCCCGGCGCGTTAACG
CGCGTGCTACACCTGGCGGCTCATCCTCTGGGGCCGTCAATGCTGAAGCTCATCAATGAAATGGC
GCGCACCACCGATATGCTGTGCCGCGAACTCCCCCAGGCATTTAACGATCTGGCCGTCGATGGCG
TCATTGTTGATCAAATGGAACCGGCAGGCGCGCTCGTTGCTGAAGCACTGGGACTGCCGTTTATC
TCTGTCGCCTGCGCGCTGCCTCTCAATCGTGAACCGGATATGCCCCTGGCGGTTATGCCTTTCGA
ATACGGGACCAGCGACGCGGCTCGCGAACGTTATGCCGCCAGTGAAAAAATTTATGACTGGCTAA
TGCGTCGTCATGACCGTGTCATTGCCGAACACAGCCACAGAATGGGCTTAGCCCCCCCGGCAAAA
GCTTCACCAGTGTTTTTCGCCACTGGCGCAAATCAGCCAGCTTGTTCCTGAACTGGATTTTCCCC
GCAAAGCGTTACCGGCTTGTTTTCATGCCGTCGGGCCTCTGCGCGAAACGCACGCACCGTCAACG
TCTTCATCCCGTTATTTTACATCCTCAGAAAAACCCCGGATTTTCGCCTCGCTGGGCACGCTTCA
GGGACACCGTTATGGGCTGTTTAAAACGATAGTGAAAGCCTGTGAAGAAATTGACGGTCAGCTCC
TGTTAGCCCACTGTGGTCGTCTTACGGACTCTCAGTGTGAAGAGCTGGCGCGAAGCCGTCATACA
CAGGTGGTGGATTTTGCCGATCAGTCAGCCGCGCTGTCTCAGGCGCAGCTGGCGATCACCCACGG
CGGCATGAATACGGTACTGGACGCGATTAATTACCGGACGCCCCTTTTAGCGCTTCCGCTGGCCT
TTGATCAGCCCGGCGTCGCGTCACGCATCGTTTATCACGGCATCGGCAAGCGTGCTTCCCGCTTT
ACCACCAGCCATGCTTTGGCTCGTCAGATGCGTTCATTGCTGACCAACGTCGACTTTCAGCAGCG
CATGGCGAAAATCCAGACAGCCCTTCGTTTGGCAGGGGGCACCATGGCCGCTGCCGATATCATTG
AGCAGGTTATGTGCACCGGTCAGCCTGTCTTAAGTGGGAGCGGCTATGCAACCGCATTATGATCT
GATTCTCGTGGGGGCTGGACTCGCGAATGGCCTTATCGCCCTGCGTCTTCAGCAGCAGCAACCTG
ATATGCGTATTTTGCTTATCGACGCCGCACCCCAGGCGGGCGGGAATCATACGTGGTCATTTCAC
CACGATGATTTGACTGAGAGCCAACATCGTTGGATAGCTCCGCTGGTGGTTCATCACTGGCCCGA
CTATCAGGTACGCTTTCCCACACGCCGTCGTAAGCTGAACAGCGGCTACTTTTGTATTACTTCTC
AGCGTTTCGCTGAGGTTTTACAGCGACAGTTTGGCCCGCACTTGTGGATGGATACCGCGGTCGCA
GAGGTTAATGCGGAATCTGTTCGGTTGAAAAAGGGTCAGGTTATCGGTGCCCGCGCGGTGATTGA
CGGGCGGGGTTATGCGGCAAATTCAGCACTGAGCGTGGGCTTCCAGGCGTTTATTGGCCAGGAAT
GGCGATTGAGCCACCCGCATGGTTTATCGTCTCCCATTATCATGGATGCCACGGTCGATCAGCAA
AATGGTTATCGCTTCGTGTACAGCCTGCCGCTCTCGCCGACCAGATTGTTAATTGAAGACACGCA
CTATATTGATAATGCGACATTAGATCCTGAATGCGCGCGGCAAAATATTTGCGACTATGCCGCGC
AACAGGGTTGGCAGCTTCAGACACTGCTGCGAGAAGAACAGGGCGCCTTACCCATTACTCTGTCG
GGCAATGCCGACGCATTCTGGCAGCAGCGCCCCCTGGCCTGTAGTGGATTACGTGCCGGTCTGTT
CCATCCTACCACCGGCTATTCACTGCCGCTGGCGGTTGCCGTGGCCGACCGCCTGAGTGCACTTG
ATGTCTTTACGTCGGCCTCAATTCACCATGCCATTACGCATTTTGCCCGCGAGCGCTGGCAGCAG
CAGGGCTTTTTCCGCATGCTGAATCGCATGCTGTTTTTAGCCGGACCCGCCGATTCACGCTGGCG
GGTTATGCAGCGTTTTTATGGTTTACCTGAAGATTTAATTGCCCGTTTTTATGCGGGAAAACTCA
CGCTGACCGATCGGCTACGTATTCTGAGCGGCAAGCCGCCTGTTCCGGTATTAGCAGCATTGCAA
GCCATTATGACGACTCATCGTTAAAGAGCGACTACATGAAACCAACTACGGTAATTGGTGCAGGC
TTCGNTGGCCTGGCACTGGCAATTCGTCTACAAGCTGCGGGGATCCCCGTCTTACTGCTTGAACA
ACGTGATAAACCCGGCGGTCGGGCTTATGTCTACGAGGATCAGGGGTTTACCTTTGATGCAGGCC
CGACGGTTATCACCGATCCCAGTGCCATTGAAGAACTGTTTGCACTGGCAGGAAAACAGTTAAAA
GAGTATGTCGAACTGCTGCCGGTTACGCCGTTTTACCGCCTGTGTTGGGAGTCAGGGAAGGTCTT
TAATTACGATAACGATCAAACCCGGCTCGAAGCGCAGATTCAGCAGTTTAATCCCCGCGATGTCG
AAGGTTATCGTCAGTTTCTGGACTATTCACGCGCGGTGTTTAAAGAAGGCTATCTAAAGCTCGGT
ACTGTCCCTTTTTTATCGTTCAGAGACATGCTTCGCGCCGCACCTCAACTGGCGAAACTGCAGGC
ATGGAGAAGCGTTTACAGTAAGGTTGCCAGTTACATCGAAGATGAACATCTGCGCCAGGCGTTTT
CTTTCCACTCGCTGTTGGTGGGCGGCAATCCCTTCGCCACCTCATCCATTTATACGTTGATACAC
GCGCTGGAGCGTGAGTGGGGCGTCTGGTTTCCGCGTGGCGGCACCGGCGCATTAGTTCAGGGGAT
GATAAAGCTGTTTCAGGATCTGGGTGGCGAAGTCGTGTTAAACGCCAGAGTCAGCCATATGGAAA
CGACAGGAAACAAGATTGAAGCCGTGCATTTAGAGGACGGTCGCAGGTTCCTGACGCAAGCCGTC
GCGTCAAATGCAGATGTGGTTCATACCTATCGCGACCTGTTAAGCCAGCACCCTGCCGCGGTTAA
GCAGTCCAACAAACTGCAGACTAAGCGCATGAGTAACTCTCTGTTTGTGCTCTATTTTGGTTTGA
ATCACCATCATGATCAGCTCGCGCATCACACGGTTTGTTTCGGCCCGCGTTACCGCGAGCTGATT
GACGAAATTTTTAATCATGATGGCCTCGCAGAGGACTTCTCACTTTATCTGCACGCGCCCTGTGT
CACGGATTCGTCACTGGCGCCTGAAGGTTGCGGCAGTTACTATGTGTTGGCGCCGGTGCCGCATT
TAGGCACCGCGAACCTCGACTGGACGGTTGAGGGGCCAAAACTACGCGACCGTATTTTTGCGTAC
CTTGAGCAGCATTACATGCCTGGCTTACGGAGTCAGCTGGTCACGCACCGGATGTTTACGCCGTT
TGATTTTCGCGACCAGCTTAATGCCTATCATGGCTCAGCCTTTTCTGTGGAGCCCGTTCTTACCC
AGAGCGCCTGGTTTCGGCCGCATAACCGCGATAAAACCATTACTAATCTCTACCTGGTCGGCGCA
GGCACGCATCCCGGCGCAGGCATTCCTGGCGTCATCGGCTCGGCAAAAGCGACAGCAGGTTTGAT
GCTGGAGGATCTGATATGAATAATCCGTCGTTACTCAATCATGCGGTCGAAACGATGGCAGTTGG
CTCGAAAAGTTTTGCGACAGCCTCAAAGTTATTTGATGCAAAAACCCGGCGCAGCGTACTGATGC
TCTACGCCTGGTGCCGCCATTGTGACGATGTTATTGACGATCAGACGCTGGGCTTTCAGGCCCGG
CAGCCTGCCTTACAAACGCCCGAACAACGTCTGATGCAACTTGAGATGAAAACGCGCCAGGCCTA
TGCAGGATCGCAGATGCACGAACCGGCGTTTGCGGCTTTTCAGGAAGTGGCTATGGCTCATGATA
TCGCCCCGGCTTACGCGTTTGATCATCTGGAAGGCTTCGCCATGGATGTACGCGAAGCGCAATAC
AGCCAACTGGATGATACGCTGCGCTATTGCTATCACGTTGCAGGCGTTGTCGGCTTGATGATGGC
GCAAATCATGGGCGTGCGGGATAACGCCACGCTGGACCGCGCCTGTGACCTTGGGCTGGCATTTC
AGTTGACCAATATTGCTCGCGATATTGTGGACGATGCGCATGCGGGCCGCTGTTATCTGCCGGCA
AGCTGGCTGGAGCATGAAGGTCTGAACAAAGAGAATTATGCGGCACCTGAAAACCGTCAGGCGCT
GAGCCGTATCGCCCGTCGTTTGGTGCAGGAAGCAGAACCTTACTATTTGTCTGCCACAGCCGGCC
TGGCAGGGTTGCCCCTGCGTTCCGCCTGGGCAATCGCTACGGCGAAGCAGGTTTACCGGAAAATA
GGTGTCAAAGTTGAACAGGCCGGTCAGCAAGCCTAGGATCAGCGGCAGTCAACGACCACGCCCGA
AAAATTAACGCTGCTGCTGGCCGCCTCTGGTCAGGCCCTTACTCCCGGATGCGGGCTCATCCTCC
CCGCCCTGCGCATCTCTGGCAGCGCCCGCTCTAGCGCCaTGTCTTTCCCGGAGCGTCAGATCTGC
TTGGAGCCACCCGCAGTTCGAAAAATAATAAGCTTGACCTGTGAAGTGAAAAATGGCGCACATTG
TGCGACATTTTTTTTGTCTGCCGTTTACCGCTACTGCGTCACGGATCTCCACGCGCCCTGTAGCG
GCGCATTAAGCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTCGACCCCAAAAAAC
TTGATTAGGGTGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCAACCCTATCTC
GGTCTATTCTTTTGATTTATAAGGGATTTTGCCGATTTCGGCCTATTGGTTAAAAAATGAGCTGA
TTTAACAAAAATTTAACGCGAATTTTAACAAAATCTCGAATTCAAGAAACCAATTGTCCATATTG
CATCAGACATTGCCGTCACTGCGTCTTTTACTGGCTCTTCTCGCTAACCAAACCGGTAACCCCGC
TTATTAAAAGCATTCTGTAACAAAGCGGGACCAAAGCCATGACAAAAACGCGTAACAAAAGTGTC
TATAATCACGGCAGAAAAGTCCACATTGATTATTTGCACGGCGTCACACTTTGCTATGCCATAGC
ATTTTTATCCATAAGATTAGCGGATCCTACCTGACGCTTTTTATCGCAACTCTCTACTGTTTCTC
CATACCCGTTTTTTTGGGCTAGCTAAGGAGGAGACCCCATGGGTTTGATGACCGTATTTTctTcT
GCACCTGAACTTGCCCtTcTCGGATCAACCTTTGCCCAGGTCGATCCTTCAAACTTATCGGTCTC
AGATTCGcTGACCTATGGTCAGTTCAATCTGGTTTACAACGCTTTCTCGTTTGCCATCGCGGCAA
TGTTCGCATCTGCCCTCTTCTTCTTCAGCGCTCAGGCACTCGTCGGTCAACGATACCGGTTGGCC
TTGCTTGTTTCAGCAATTGTTGTGAGTATCGCTGGGTACCACTACTTTCGGATCTTCAATAGTTG
GGATGCTGCCTACGTTCTGGAGAATGGCGTGTATTCCCTGACTAGCGAAAAATTCAACGACGCCT
ACCGCTATGTGGATTGGCTGTTGACCGTGCCTCTGTTGCTGGTGGAGACAGTGGCAGTGCTGACG
TTGCCTGCAAAGGAGGCAAGACCCTTGCTGATCAAACTGACGGTGGCTTCAGTTCTGATGATTGC
CACGGGCTACCCCGGCGAGATTTCTGACGACATTACGACTCGCATCATCTGGGGTACGGTCAGCA
CGATTCCCTTCGCCTACATCCTCTATGTGTTGTGGGTCGAACTGTCCAGGTCCCTTGTCCGCCAG
CCCGCTGCTGTACAAACCCTGGTCCGCAACATGCGGTGGCTGCTGTTGCTCTCCTGGGGTGTTTA
CCCGATCGCATACCTTCTACCCATGCTTGGAGTATCCGGTACGTCCGCGGCTGTCGGCGTTCAGG
TTGGCTATACGATCGCAGACGTGCTGGCGAAGCCTGTATTTGGTCTTCTAGTCTTCGCGATTGCA
CTCGTGAAAACAAAAGCAGATCAAGAAAGCAGTGAACCACATGCCGCAATAGGTGCTGCTGCAAA
TAAATCGGGAGGCAGTCTTATCTCCTAGCGGCCGCAGATCTGCTTGGAGCCACCCGCAGTTCGAA
AAATAATAAGCTTGACCTGTGAAGTGAAAAATGGCGCACATTGTGCGACATTTTTTTTGTCTGCC
GTTTACCGCTACTGCGTCACAGATCTGAATTCACTGGCCGTCGTTTTACAACGTCGTGACTGGGA
AAACCCTGGCGTTACCCAACTTAATCGCCTTGCAGCACATCCCCCTTTCGCCAGCTGGCGTAATA
GCGAAGAGGCCCGCACCGATCGCCCTTCCCAACAGTTGCGCAGCCTGAATGGCGAATGGCGCCTG
ATGCGGTATTTTCTCCTTACGCATCTGTGCGGTATTTCACACCGCATATGGTGCACTCTCAGTAC
AATCTGCTCTGATGCCGCATAGTTAAGCCAGCCCCGACACCCGCCAACACCCGCTGACGCGCCCT
GACGGGCTTGTCTGCTCCCGGCATCCGCTTACAGACAAGCTGTGACCGTCTCCGGGAGCTGCATG
TGTCAGAGGTTTTCACCGTCATCACCGAAACGCGCGAGACGAAAGGGCCTCGTGATACGCCTATT
TTTATAGGTTAATGTCATGATAATAATGGTTTCTTAGCACCCTTTCTCGGTCCTTCAACGTTCCT
GACAACGAGCCTCCTTTTCGCCAATCCATCGACAATCACCGCGAGTCCCTGCTCGAACGCTGCGT
CCGGACCGGCTTCGTCGAAGGCGTCTATCGCGGCCCGCAACAGCGGCGAGAGCGGAGCCTGTTCA
ACGGTGCCGCCGCGCTCGCCGGCATCGCTGTCGCCGGCCTGCTCCTCAAGCACGGCCCCAACAGT
GAAGTAGCTGATTGTCATCAGCGCATTGACGGCGTCCCCGGCCGAAAAACCCGCCTCGCAGAGGA
AGCGAAGCTGCGCGTCGGCCGTTTCCATCTGCGGTGCGCCCGGTCGCGTGCCGGCATGGATGCGC
GCGCCATCGCGGTAGGCGAGCAGCGCCTGCCTGAAGCTGCGGGCATTCCCGATCAGAAATGAGCG
CCAGTCGTCGTCGGCTCTCGGCACCGAATGCGTATGATTCTCCGCCAGCATGGCTTCGGCCAGTG
CGTCGAGCAGCGCCCGCTTGTTCCTGAAGTGCCAGTAAAGCGCCGGCTGCTGAACCCCCAACCGT
TCCGCCAGTTTGCGTGTCGTCAGACCGTCTACGCCGACCTCGTTCAACAGGTCCAGGGCGGCACG
GATCACTGTATTCGGCTGCAACTTTGTCATGATTGACACTTTATCACTGATAAACATAATATGTC
CACCAACTTATCAGTGATAAAGAATCCGCGCGTTCAATCGGACCAGCGGAGGCTGGTCCGGAGGC
CAGACGTGAAACCCAACATACCCCTGATCGTAATTCTGAGCACTGTCGCGCTCGACGCTGTCGGC
ATCGGCCTGATTATGCCGGTGCTGCCGGGCCTCCTGCGCGATCTGGTTCACTCGAACGACGTCAC
CGCCCACTATGGCATTCTGCTGGCGCTGTATGCGTTGGTGCAATTTGCCTGCGCACCTGTGCTGG
GCGCGCTGTCGGATCGTTTCGGGCGGCGGCCAATCTTGCTCGTCTCGCTGGCCGGCGCCACTGTC
GACTACGCCATCATGGCGACAGCGCCTTTCCTTTGGGTTCTCTATATCGGGCGGATCGTGGCCGG
CATCACCGGGGCGACTGGGGCGGTAGCCGGCGCTTATATTGCCGATATCACTGATGGCGATGAGC
GCGCGCGGCACTTCGGCTTCATGAGCGCCTGTTTCGGGTTCGGGATGGTCGCGGGACCTGTGCTC
GGTGGGCTGATGGGCGGTTTCTCCCCCCACGCTCCGTTCTTCGCCGCGGCAGCCTTGAACGGCCT
CAATTTCCTGACGGGCTGTTTCCTTTTGCCGGAGTCGCACAAAGGCGAACGCCGGCCGTTACGCC
GGGAGGCTCTCAACCCGCTCGCTTCGTTCCGGTGGGCCCGGGGCATGACCGTCGTCGCCGCCCTG
ATGGCGGTCTTCTTCATCATGCAACTTGTCGGACAGGTGCCGGCCGCGCTTTGGGTCATTTTCGG
CGAGGATCGCTTTCACTGGGACGCGACCACGATCGGCATTTCGCTTGCCGCATTTGGCATTCTGC
ATTCACTCGCCCAGGCAATGATCACCGGCCCTGTAGCCGCCCGGCTCGGCGAAAGGCGGGCACTC
ATGCTCGGAATGATTGCCGACGGCACAGGCTACATCCTGCTTGCCTTCGCGACACGGGGATGGAT
GGCGTTCCCGATCATGGTCCTGCTTGCTTCGGGTGGCATCGGAATGCCGGCGCTGCAAGCAATGT
TGTCCAGGCAGGTGGATGAGGAACGTCAGGGGCAGCTGCAAGGCTCACTGGCGGCGCTCACCAGC
CTGACCTCGATCGTCGGACCCCTCCTCTTCACGGCGATCTATGCGGCTTCTATAACAACGTGGAA
CGGGTGGGCATGGATTGCAGGCGCTGCCCTCTACTTGCTCTGCCTGCCGGCGCTGCGTCGCGGGC
TTTGGAGCGGCGCAGGGCAACGAGCCGATCGCTGATCGTGGAAACGATAGGCCTATGCCATGCGG
GTCAAGGCGACTTCCGGCAAGCTATACGCGCCCTAGAATTGTCAATTTTAATCCTCTGTTTATCG
GCAGTTCGTAGAGCGCGCCGTGCGTCCCGAGCGATACTGAGCGAAGCAAGTGCGTCGAGCAGTGC
CCGCTTGTTCCTGAAATGCCAGTAAAGCGCTGGCTGCTGAACCCCCAGCCGGAACTGACCCCACA
AGGCCCTAGCGTTTGCAATGCACCAGGTCATCATTGACCCAGGCGTGTTCCACCAGGCCGCTGCC
TCGCAACTCTTCGCAGGCTTCGCCGACCTGCTCGCGCCACTTCTTCACGCGGGTGGAATCCGATC
CGCACATGAGGCGGAAGGTTTCCAGCTTGAGCGGGTACGGCTCCCGGTGCGAGCTGAAATAGTCG
AACATCCGTCGGGCCGTCGGCGACAGCTTGCGGTACTTCTCCCATATGAATTTCGTGTAGTGGTC
GCCAGCAAACAGCACGACGATTTCCTCGTCGATCAGGACCTGGCAACGGGACGTTTTCTTGCCAC
GGTCCAGGACGCGGAAGCGGTGCAGCAGCGACACCGATTCCAGGTGCCCAACGCGGTCGGACGTG
AAGCCCATCGCCGTCGCCTGTAGGCGCGACAGGCATTCCTCGGCCTTCGTGTAATACCGGCCATT
GATCGACCAGCCCAGGTCCTGGCAAAGCTCGTAGAACGTGAAGGTGATCGGCTCGCCGATAGGGG
TGCGCTTCGCGTACTCCAACACCTGCTGCCACACCAGTTCGTCATCGTCGGCCCGCAGCTCGACG
CCGGTGTAGGTGATCTTCACGTCCTTGTTGACGTGGAAAATGACCTTGTTTTGCAGCGCCTCGCG
CGGGATTTTCTTGTTGCGCGTGGTGAACAGGGCAGAGCGGGCCGTGTCGTTTGGCATCGCTCGCA
TCGTGTCCGGCCACGGCGCAATATCGAACAAGGAAAGCTGCATTTCCTTGATCTGCTGCTTCGTG
TGTTTCAGCAACGCGGCCTGCTTGGCCTCGCTGACCTGTTTTGCCAGGTCCTCGCCGGCGGTTTT
TCGCTTCTTGGTCGTCATAGTTCCTCGCGTGTCGATGGTCATCGACTTCGCCAAACCTGCCGCCT
CCTGTTCGAGACGACGCGAACGCTCCACGGCGGCCGATGGCGCGGGCAGGGCAGGGGGAGCCAGT
TGCACGCTGTCGCGCTCGATCTTGGCCGTAGCTTGCTGGACCATCGAGCCGACGGACTGGAAGGT
TTCGCGGGGCGCACGCATGACGGTGCGGCTTGCGATGGTTTCGGCATCCTCGGCGGAAAACCCCG
CGTCGATCAGTTCTTGCCTGTATGCCTTCCGGTCAAACGTCCGATTCATTCACCCTCCTTGCGGG
ATTGCCCCGACTCACGCCGGGGCAATGTGCCCTTATTCCTGATTTGACCCGCCTGGTGCCTTGGT
GTCCAGATAATCCACCTTATCGGCAATGAAGTCGGTCCCGTAGACCGTCTGGCCGTCCTTCTCGT
ACTTGGTATTCCGAATCTTGCCCTGCACGAATACCAGCTCCGCGAAGTCGCTCTTCTTGATGGAG
CGCATGGGGACGTGCTTGGCAATCACGCGCACCCCCCGGCCGTTTTAGCGGCTAAAAAAGTCATG
GCTCTGCCCTCGGGCGGACCACGCCCATCATGACCTTGCCAAGCTCGTCCTGCTTCTCTTCGATC
TTCGCCAGCAGGGCGAGGATCGTGGCATCACCGAACCGCGCCGTGCGCGGGTCGTCGGTGAGCCA
GAGTTTCAGCAGGCCGCCCAGGCGGCCCAGGTCGCCATTGATGCGGGCCAGCTCGCGGACGTGCT
CATAGTCCACGACGCCCGTGATTTTGTAGCCCTGGCCGACGGCCAGCAGGTAGGCCTACAGGCTC
ATGCCGGCCGCCGCCGCCTTTTCCTCAATCGCTCTTCGTTCGTCTGGAAGGCAGTACACCTTGAT
AGGTGGGCTGCCCTTCCTGGTTGGCTTGGTTTCATCAGCCATCCGCTTGCCCTCATCTGTTACGC
CGGCGGTAGCCGGCCAGCCTCGCAGAGCAGGATTCCCGTTGAGCACCGCCAGGTGCGAATAAGGG
ACAGTGAAGAAGGAACACCCGCTCGCGGGTGGGCCTACTTCACCTATCCTGCCCGGCTGACGCCG
TTGGATACACCAAGGAAAGTCTACACGAACCCTTTGGCAAAATCCTGTATATCGTGCGAAAAAGG
ATGGATATACCGAAAAAATCGCTATAATGACCCCGAAGCAGGGTTATGCAGCGGAAAAGATCCGT
CGACCCTTTCCGACGCTCACCGGGCTGGTTGCCCTCGCCGCTGGGCTGGCGGCCGTCTATGGCCC
TGCAAACGCGCCAGAAACGCCGTCGAAGCCGTGTGCGAGACACCGCGGCCGCCGGCGTTGTGGAT
ACCTCGCGGAAAACTTGGCCCTCACTGACAGATGAGGGGCGGACGTTGACACTTGAGGGGCCGAC
TCACCCGGCGCGGCGTTGACAGATGAGGGGCAGGCTCGATTTCGGCCGGCGACGTGGAGCTGGCC
AGCCTCGCAAATCGGCGAAAACGCCTGATTTTACGCGAGTTTCCCACAGATGATGTGGACAAGCC
TGGGGATAAGTGCCCTGCGGTATTGACACTTGAGGGGCGCGACTACTGACAGATGAGGGGCGCGA
TCCTTGACACTTGAGGGGCAGAGTGCTGACAGATGAGGGGCGCACCTATTGACATTTGAGGGGCT
GTCCACAGGCAGAAAATCCAGCATTTGCAAGGGTTTCCGCCCGTTTTTCGGCCACCGCTAACCTG
TCTTTTAACCTGCTTTTAAACCAATATTTATAAACCTTGTTTTTAACCAGGGCTGCGCCCTGTGC
GCGTGACCGCGCACGCCGAAGGGGGGTGCCCCCCCTTCTCGAACCCTCCCGGCCCGCTAACGCGG
GCCTCCCATCCCCCCAGGGGCTGCGCCCCTCGGCCGCGAACGGCCTCACCCCAAAAATGGCAGCC
AAGCTGACCACTTCTGCGCTCGGCCCTTCCGGCTGGCTGGTTTATTGCTGATAAATCTGGAGCCG
GTGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCAAAGGATCTTC
TTGAGATCCTTTTTTTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGAACTCTGTAGC
ACCGCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACGGGG
GGTTCGTGCACACAGCCCAGCTTGGAGCGAACGACCTACACCGAACTGAGATACCTACAGCGTGA
GCTATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTC.
(pLO11-blhDxrCRT-GR plasmid sequence)

According to another aspect of the invention there is provided a bacterial cell comprising the nucleic acid of the invention.

The nucleic acid of the invention may be stably integrated on the chromosome, or may be extrachromosomal.

According to another aspect of the invention there is provided a method of modifying a bacterial cell to fix CO₂ and/or produce an organic product, the method comprising the step of transforming the bacterial cell with the nucleic acid according to the invention.

The bacterial cell may further be provided with an electron source, e.g. as described herein. The bacterial cell may be further transformed with nucleic acid, such as plamids, described herein. For example, for the expression of the MtrCAB and/or carbonic anhydrase. One or more, or all of the genes may be provided for transformation on the same plasmid.

According to another aspect of the invention there is provided an organic product produced from the electromicrobial system or method of the invention herein.

Where reference is made to a polypeptide or nucleotide sequence, such as a variant polypeptide or nucleotide sequence, the skilled person will understand that one or more amino acid residue or nucleotide substitutions, deletions or additions, may be tolerated, optionally two substitutions may be tolerated in the sequence, such that it maintains its function. The skilled person will appreciate that 1, 2, 3, 4, 5 or more amino acid residues or nucleotides may be substituted, added or removed without affecting function. References to sequence identity may be determined by BLAST sequence alignment (www.ncbi.nlm.nih.gov/BLAST/) using standard/default parameters. For example, the sequence may have 99% identity and still function according to the invention. In other embodiments, the sequence may have 98% identity and still function according to the invention. In another embodiment, the sequence may have 95% identity and still function according to the invention. In another embodiment, the sequence may have 90%, 85%, or 80% identity and still function according to the invention. In one embodiment, the variation and sequence identity may be according the full length sequence. In other embodiments, the variation may be limited to non-conserved sequences and/or sequences outside of active sites, such as binding domains. Therefore, an active site or binding site of a protein may be 100% identical, whereas the flanking sequences may comprise the stated variations in identity. Such variants may be termed “conserved active site variants”.

Amino acid substitutions may be conservative substitutions. For example, a modified residue may comprise substantially similar properties as the wild-type substituted residue. For example, a substituted residue may comprise substantially similar or equal charge or hydrophobicity as the wild-type substituted residue. For example, a substituted residue may comprise substantially similar molecular weight or steric bulk as the wild-type substituted residue. With reference to “variant” nucleic acid sequences, the skilled person will appreciate that 1, 2, 3, 4, 5 or more codons may be substituted, added or removed without affecting function. For example, conservative substitutions may be considered.

The skilled person will appreciate that reference to a bacterial cell may refer to one or more cells, such as a plurality of cells, or a culture of cells.

The skilled person will appreciate that preferred features of any one embodiment and/or aspect of the invention may be applied to all other embodiments and/or aspects of the invention.

Examples embodying an aspect of the invention will now be described with reference to the following figures:

FIG. 1. A rhodopsin-based photoautotrophic system is able to fix CO₂. Light can be used to activate rhodopsin and generate electricity. Light activated rhodopsin pumps protons, and when coupled with ATP synthase, generates ATP. An electrode, mediated by riboflavin, can serve as an electron donor to supply electrons. The closed redox loop drives CO₂ fixation.

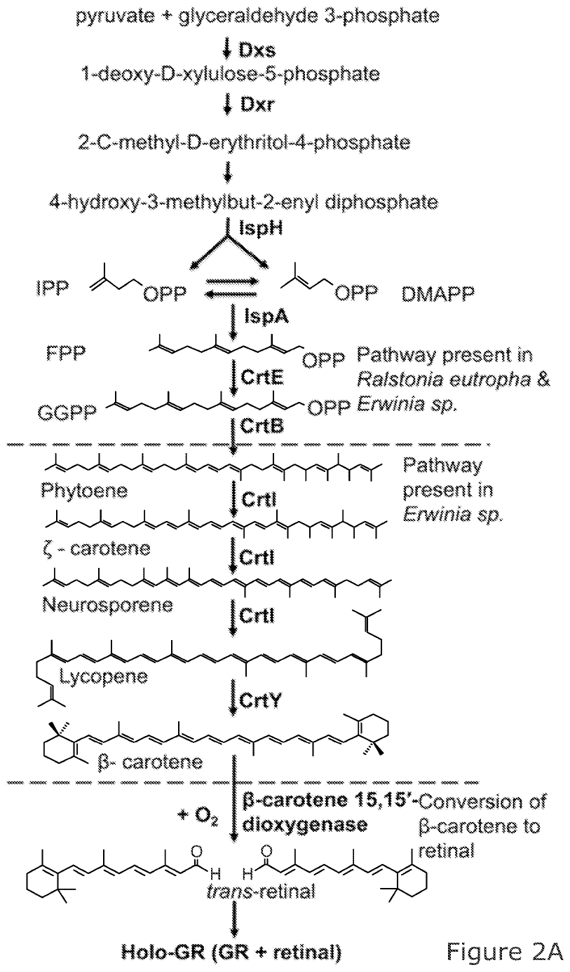

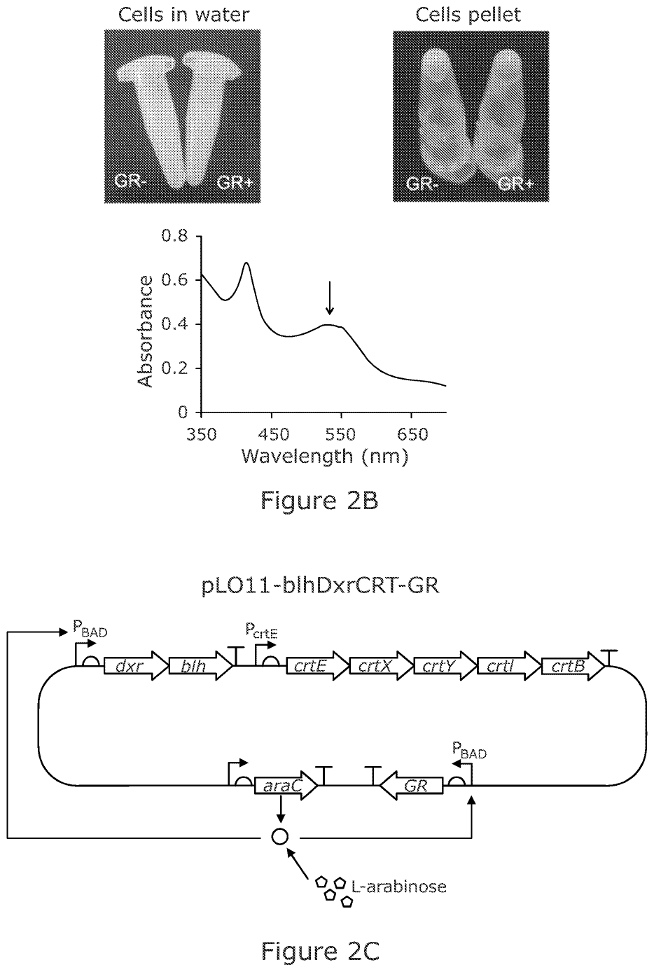

FIG. 2. (A) Pathway showing the synthesis of β-carotene from pyruvate and glyceraldehyde 3-phosphate and its conversion to retinal by β-carotene 15,15′-dioxygenase (product key: IPP, isopentenyl diphosphate; DMAPP, dimethylallyl pyrophosphate; FPP, farnesyl diphosphate; GGPP, geranylgeranyl diphosphate). The parts of the pathway common to both Ralstonia eutropha and Erwinia sp. and unique to Erwinia sp. are indicated. The enzyme key is as follows: Dxs, 1-deoxy-D-xylulose-5-phosphate synthase; Dxr, 1-deoxy-D-xylulose 5-phosphate reductoisomerase; IspH, 4-hydroxy-3-methylbut-2-enyl diphosphate reductase; crtE, geranylgeranyl diphosphate synthase; crtB, phytoene synthase; crtI, phytoene desaturase; crtY, lycopene cyclase. (B) GR expression in R. eutropha H16 causes a distinct pink colouration. Absorbance scan of isolated membranes from the induced H16 GR cells shows absorption at 540 nm (arrowed), a light wavelength that is not absorbed by chlorophyll. (C) The gene-circuit design shows the arrangement of the pLO11-blhDxrCRT-GR construct. This contains the crt operon (crtEXYIB) plus an 879 bp upstream region containing the crtE endogenous promoter (P_{crtE}) and the dxr, blh and GR genes (with upstream ribosome binding site shown as semi-circles) under the control of the arabinose inducible P_{BAD} promoter.

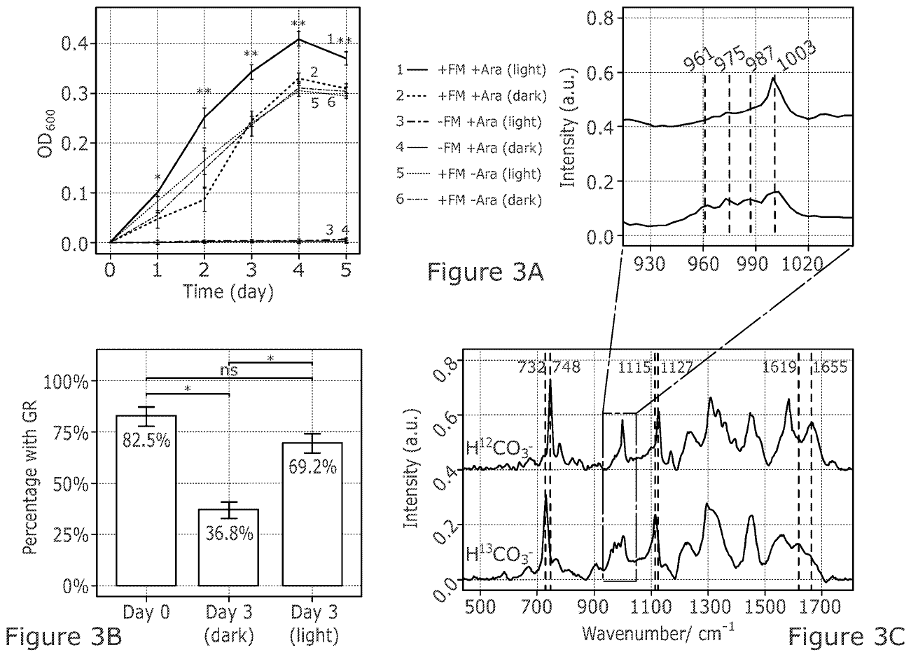

FIG. 3. (A) Growth curves, represented by OD600, of R. eutropha H16Δpha with pLO11-blhDxrCRT-GR (R. eutropha-GR) grown in conditions with and without formate, with and without light and with and without induction of GR expression. (B) Bar chart showing the percentage of the R. eutropha-GR cell population grown in the presence of formate expressing GR at day 0 and after 3 days in the light and dark. The significance *is <0.001. (C) SCRS of cells of R. eutropha-GR grown under light in either ¹²C-bicarbonate (blue) or ¹³C-bicarbonate (red). Cells grown in ¹³C-bicarbonate exhibited isotopic Raman shifts from 1655 to 1619 cm⁻¹ (Amide I of proteins), 1127 to 1115 cm⁻¹ (cytochrome c), 748 to 732 cm⁻¹ (cytochrome c) and 1002 to 987, 975 and 961 cm⁻¹ (phenylalanine).

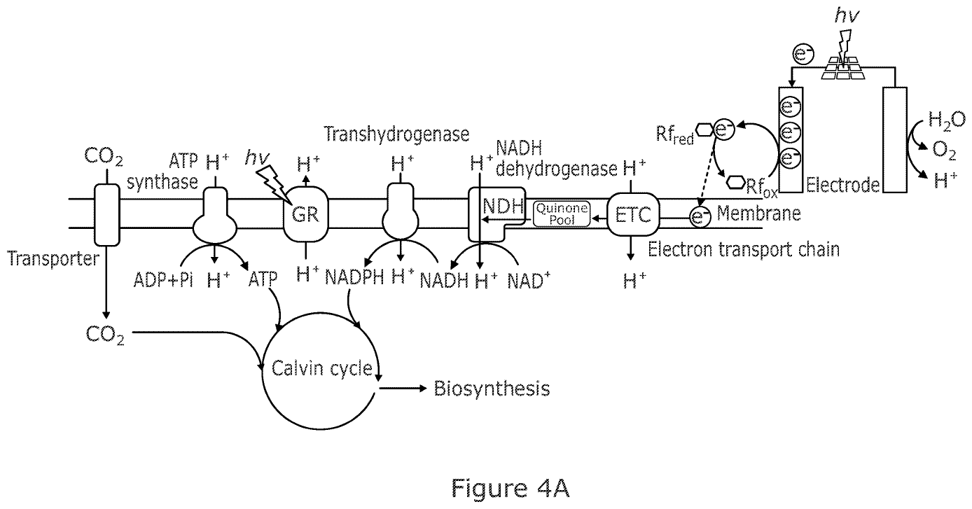

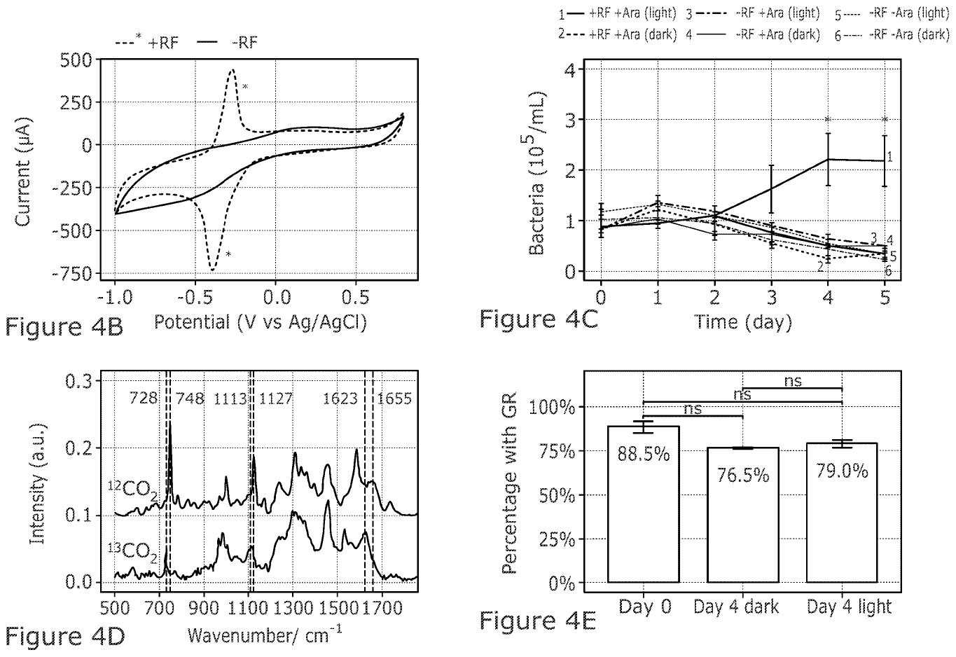

FIG. 4. (A) A photoelectrochemical CO₂ fixation bioreactor system. A light source to generate electricity. Electrons are transferred from electrode to cells mediated by riboflavin. The light activated GR system with a complete redox loop can drive autotrophic growth of bacteria via light-driven ATP synthesis. NADH is generated by an electron transfer from quinol pool to NAD⁺. Then NADH is converted into NADPH catalysed by membrane-bound transhydrogenase. (B) Cyclic voltammetry (CV) analysis of the abiotic electrochemical system with or without 50 μM riboflavin using carbon cloth as the working electrode. CVs were conducted with a scan rate of 0.05 V/s within a potential range between −1.0 V and 0.8 V versus Ag/AgCl electrode. (C) Growth of engineered R. eutropha-GR with CO₂ as the sole carbon source, driven by light activated GR and electricity (data shows means±SD, n=3). (D) SCRS of cells of photoelectroautrophic R. eutropha-GR grown on ¹²C—CO₂ (green) and ¹³C—CO₂ (red). Cells grown on ¹³C—CO₂ exhibited isotopic Raman shifts from 1655 to 1623 cm⁻¹ (Amide I of proteins), 1127 to 1113 cm⁻¹ (cytochrome c), and 748 to 728 cm⁻¹ (cytochrome c). (E) Bar chart showing the percentage of R. eutropha-GR cells that maintained high levels of GR after 4 days incubation in the light and dark, without antibiotic selection pressure.

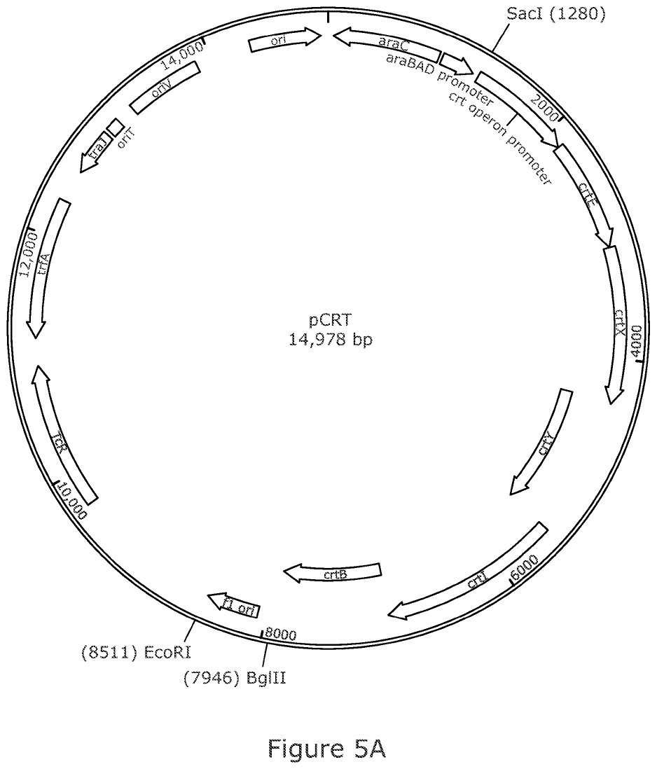

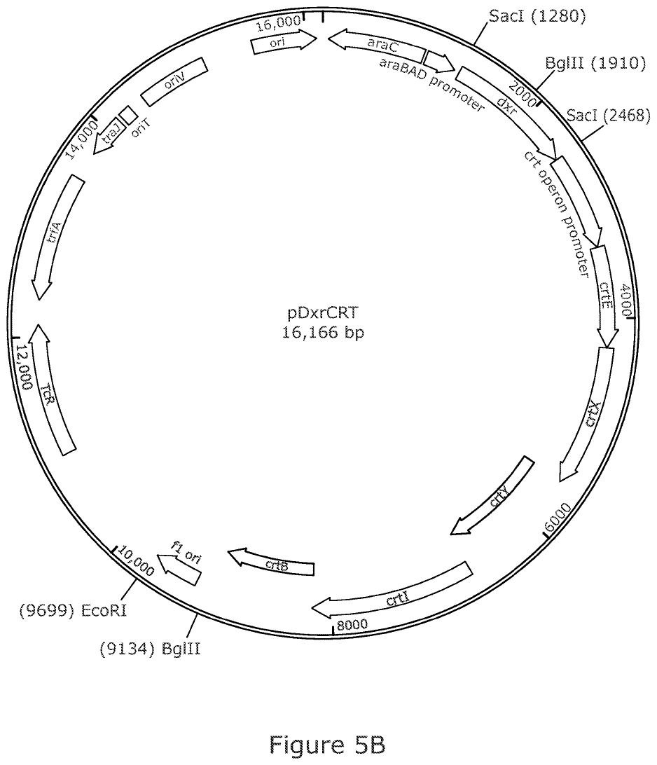

FIG. 5 Plasmid maps of (A) pCRT and (B) pDxrCRT. The genes with their associated proteins are as follows: dxr, 1-deoxy-D-xylulose 5-phosphate reductoisomerase; crtE, geranylgeranyl diphosphate synthase; crtX, zeaxanthin glucosyltransferase; crtB, phytoene synthase; crtI, phytoene desaturase; crtY, lycopene cyclase. The plasmid backbone in both cases is pLO11.

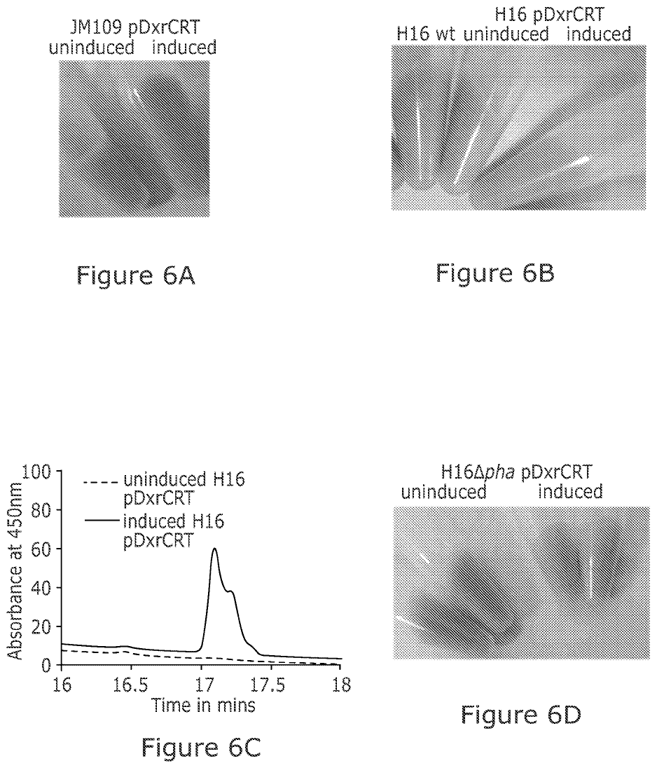

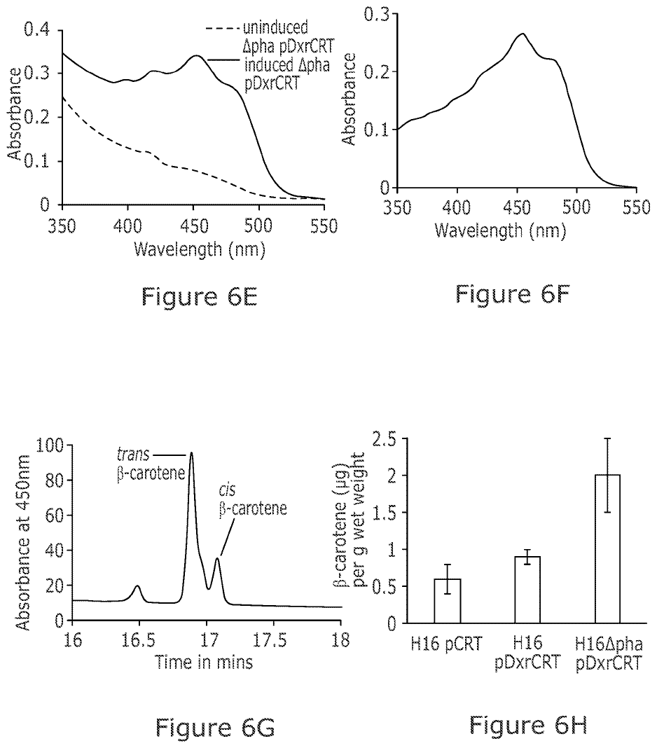

FIG. 6 (A) Cell pellets of 50 ml cultures of E. coli JM109 transformed with the pLO11-Dxr-crt (pDxrCRT) construct grown in the absence (uninduced) and presence (induced) of arabinose. (B) Cell pellets of 50 ml cultures of R. eutropha H16 wild-type cells (wt) and cells transformed with pDxrCRT grown in the presence and absence of arabinose. (C) HPLC elution profiles at 450 nm of solvent extracted uninduced (broken line) and induced (solid line) R. eutropha H16 pDxrCRT cells. (D) Cell pellets of 50 ml cultures of R. eutropha H16Δpha cells transformed with the pDxrCRT construct grown in the absence (uninduced) and presence (induced) of arabinose. (E) Absorbance scan of solvent extracted uninduced (broken line) and induced (solid line) H16Δpha pDxrCRT cells and the resulting difference absorbance scan (F). (G) HPLC elution profile at 450 nm of solvent extracted induced H16Δpha pDxrCRT cells with the trans- and cis-β-carotene peaks indicated. (H) Yield of β-carotene (expressed as μg per g of wet weight pellet) in induced R. eutropha H16 pCRT, H16 pDxrCRT and H16Δpha pDxrCRT.

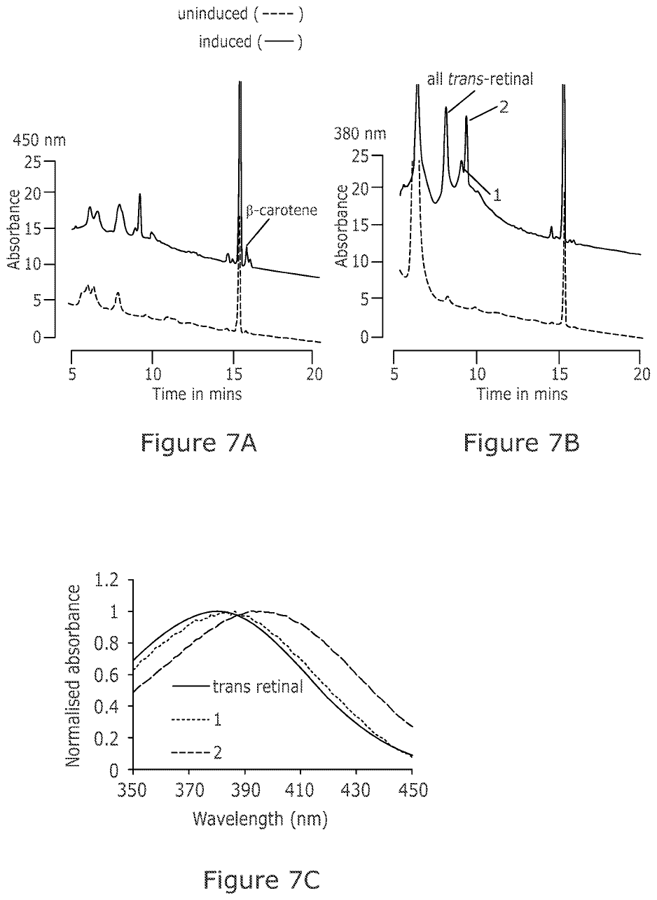

FIG. 7 HPLC elution profiles at 450 nm (A) and 380 nm (B) of solvent extracted H16Δpha blhDxrCRT-GR uninduced (solid line) and induced (broken line) cell pellets with the β-carotene and retinal peaks shown arrowed; two unknown peaks appearing after induction are labelled 1 and 2 with their normalised absorbance spectra shown compared to that of the trans-retinal peak (C).

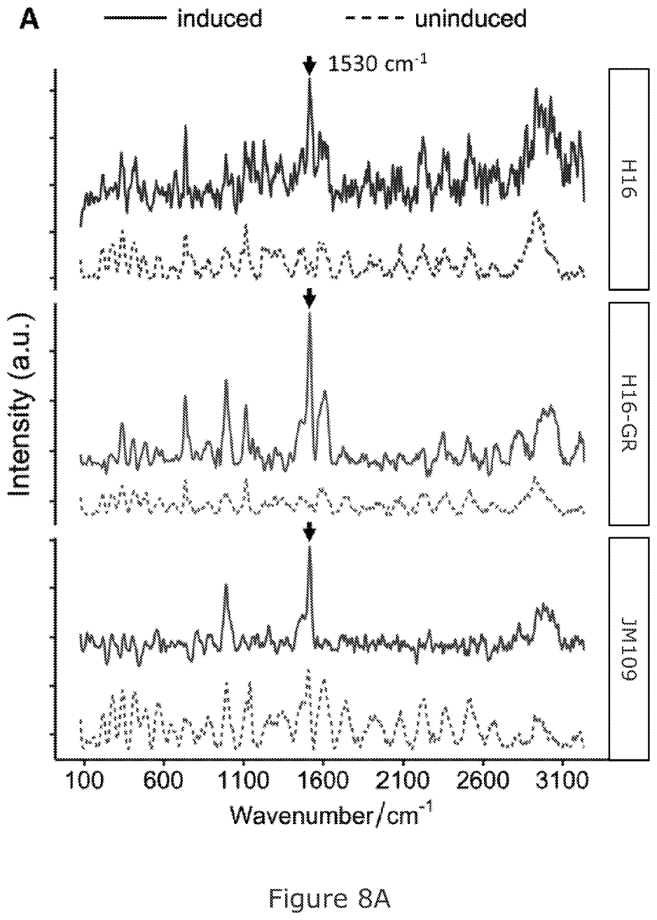

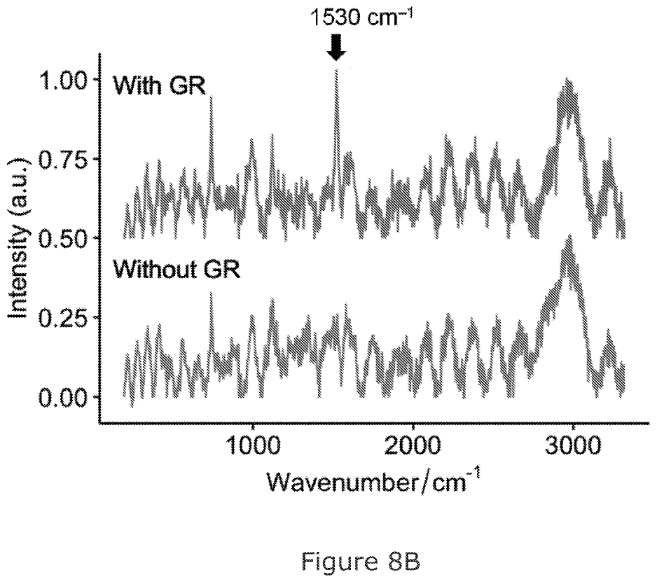

FIG. 8 (A) SCRS of GR in E. coli JM109 blhDxrCRT-GR (JM109), R. eutropha H16Δpha GR (H₁₆-GR) and R. eutropha H16Δpha blhDxrCRT-GR (H₁₆) without induction (broken line) and after induction with arabinose (solid line); strain H16Δpha GR also contained exogenous retinal. (B) SCRS of R. eutropha H16 blhDxrCRT-GR without induction (grey trace) and after induction with arabinose (green trace). The Raman signal at 1530 cm⁻¹ (arrowed) is indicative of retinal bound within GR.

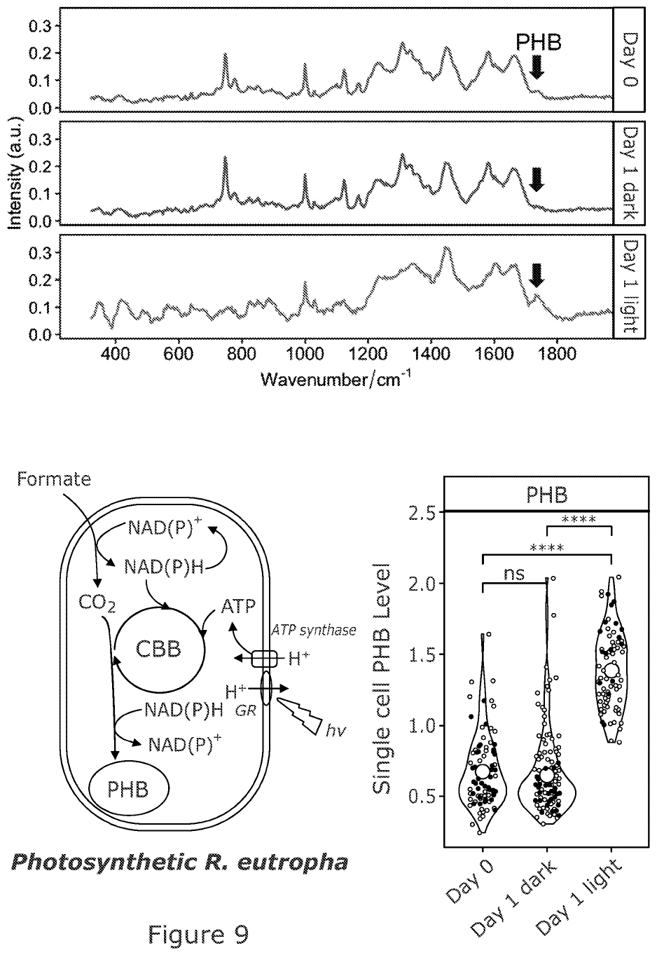

FIG. 9 (A) PHB synthesis from formate by GR-expressing R. eutropha H16 (B) Comparisons of Single-cell PHB content in the dark and light.

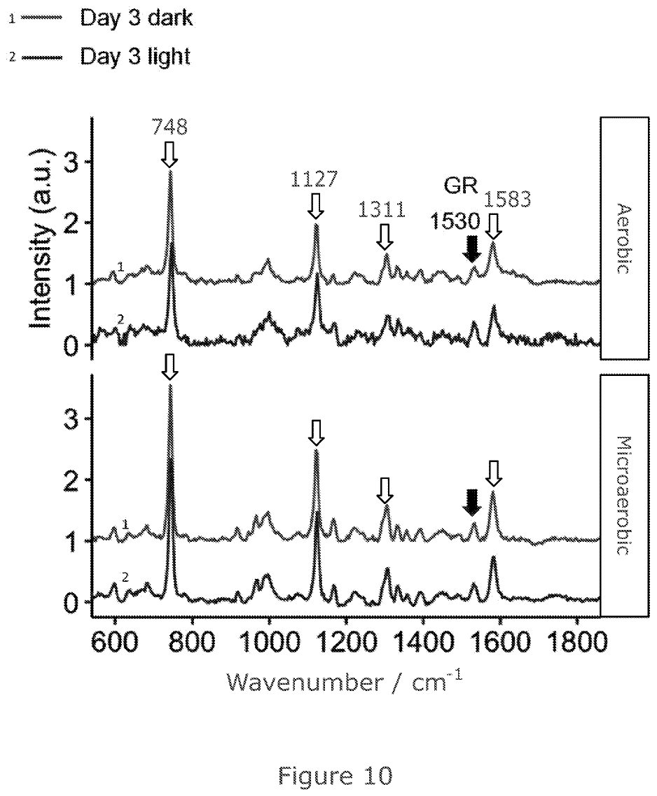

FIG. 10 SCRS of R. eutropha H16 Apha with pLO11-blhDxrCRT-GR after aerobic and microaerobic growth in MM media containing formate for 72 h under light (red line) or in the dark (blue line). Raman bands representative of the resonant cytochrome c molecules are labelled at 1583, 1311, 1123, and 750 cm⁻¹.

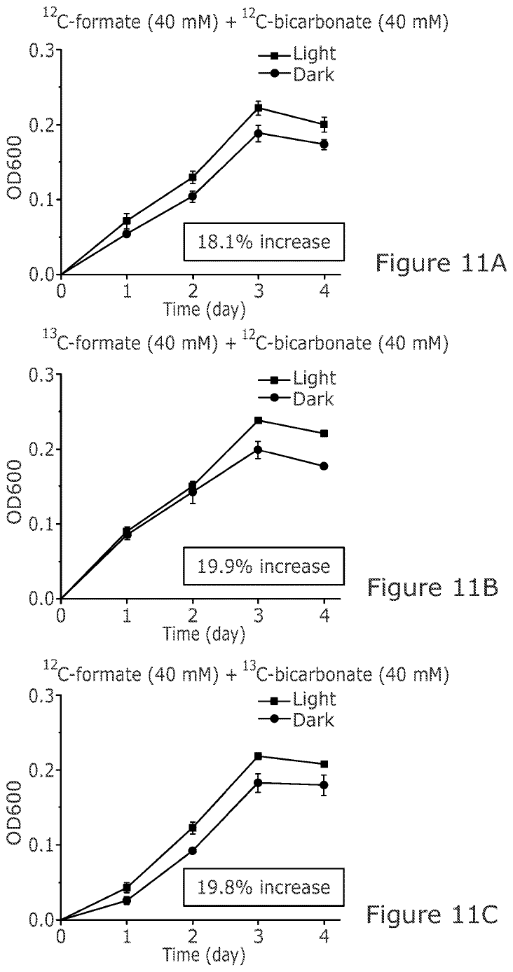

FIG. 11 Growth curves, represented by OD600, of R. eutropha H16Δpha with pLO11-blhDxrCRT-GR grown in (A) ¹²C-formate+¹²C-bicarbonate, (B) ¹³C-formate+¹²C-bicarbonate and (C) ¹²C-formate+¹³C-bicarbonate, under microaerobic conditions either in the light or in the dark. The percentage increases under light compared with the dark at the maximum growth point (day 3) are shown for each substrate condition.

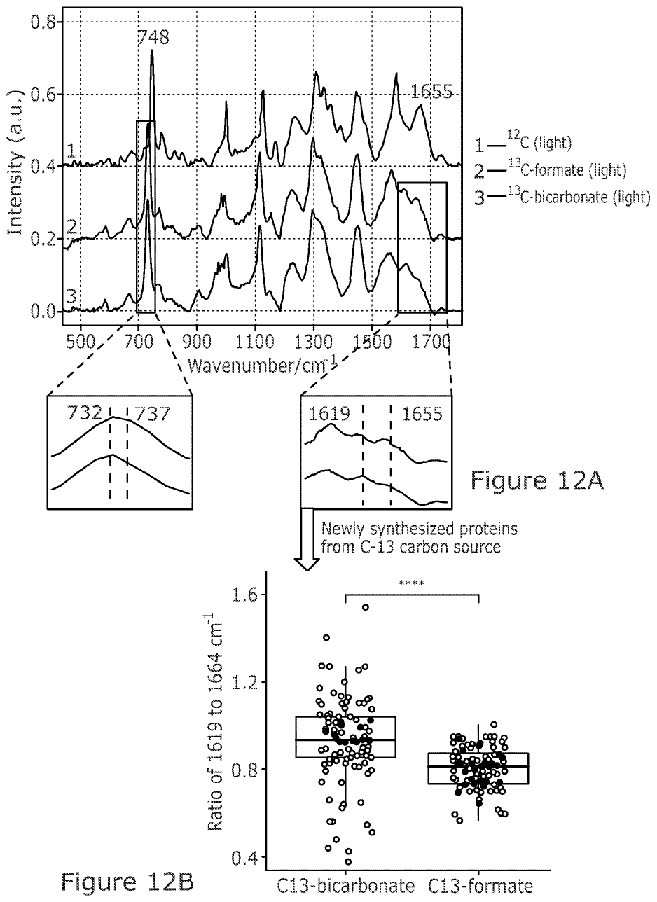

FIG. 12 (A) SCRS of R. eutropha H16Δpha with pLO11-blhDxrCRT-GR grown under light with ¹³C-formate+¹²C-bicarbonate (green) or ¹²C-formate+¹³C-bicarbonate (red). Raman shifts were observed at 737 (¹³C-formate) and 732 (¹³C-bicarbonate), and from 1664 to 1619 (both ¹³C-formate and ¹³C-bicarbonate). (B) Quantification of newly synthesised proteins at the single-cell level from ¹³C-formate (green) or ¹³C-bicarbonate (red) by calculating the ratio of the areas of the 1619-cm⁻¹ band to the 1664-cm⁻¹ band. Statistical significance was calculated based on Student's t test (p<0.0001).

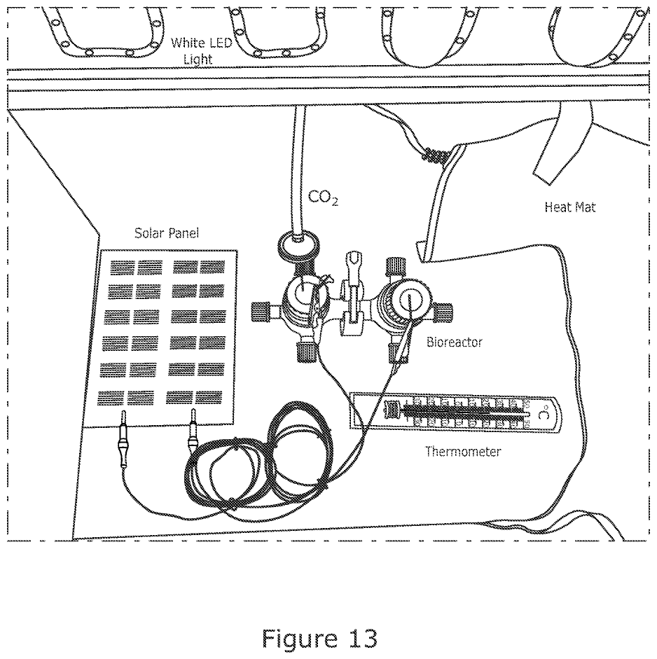

FIG. 13 The setup of photo-electrosynthetic system.

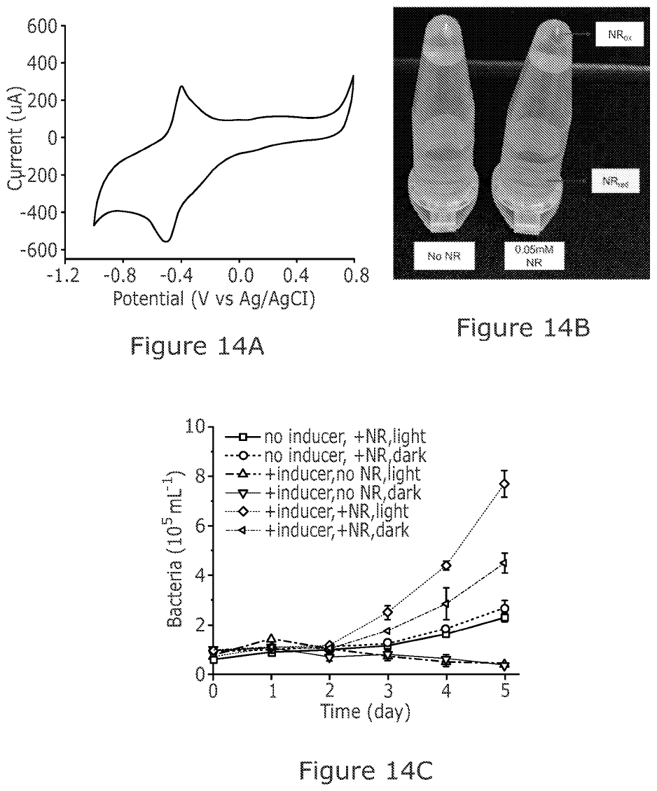

FIG. 14 (A) Cyclic voltammetry (CV) analysis of the abiotic electrochemical system with or without 50 M neutral red using carbon cloth as the working electrode. CVs were conducted with a scan rate of 0.05 V/s within a potential range between-1.0 V and 0.8 V versus Ag/AgCl electrode. (B) Color changes in the centrifuged samples. The light-yellow broth indicated the formation of reduced NR (NR_{red}) while the red biomass (at the bottom of centrifuge tubes) indicated that oxidized NR (NRₒₓ) formed inside R. eutropha. (C) Growth of engineered R. eutropha-GR with CO₂ as the sole carbon source, driven by light activated GR and electricity (data shows means±SD, n=3).

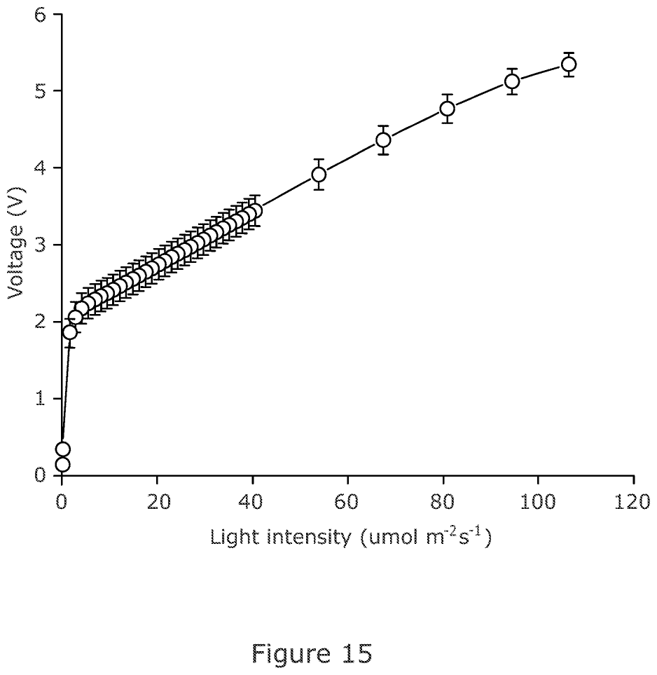

FIG. 15 The electricity production from solar panel.

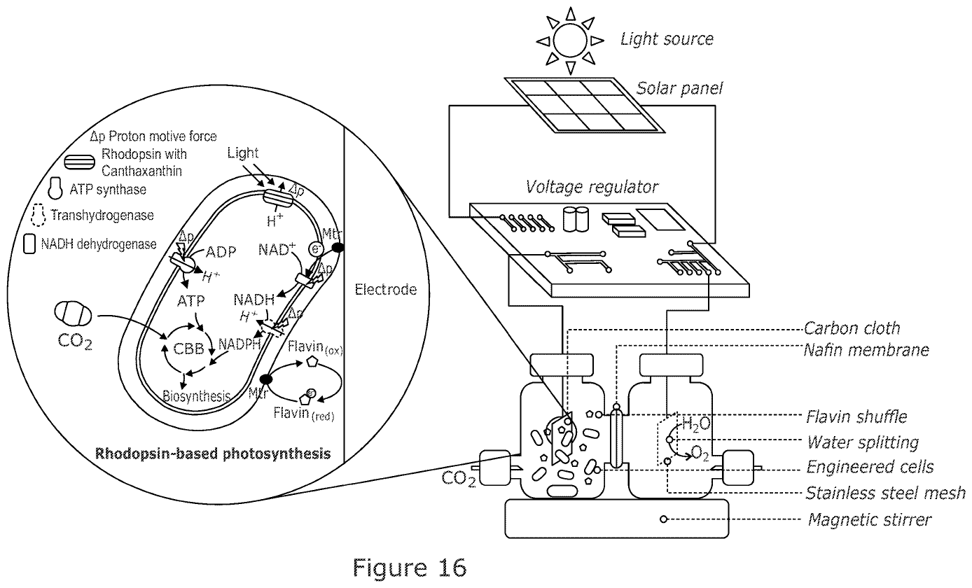

FIG. 16 Construction of hybrid photosynthesis by combining an electrochemical system with engineered cells expressing rhodopsin and an outer-membrane conduit Mtr.

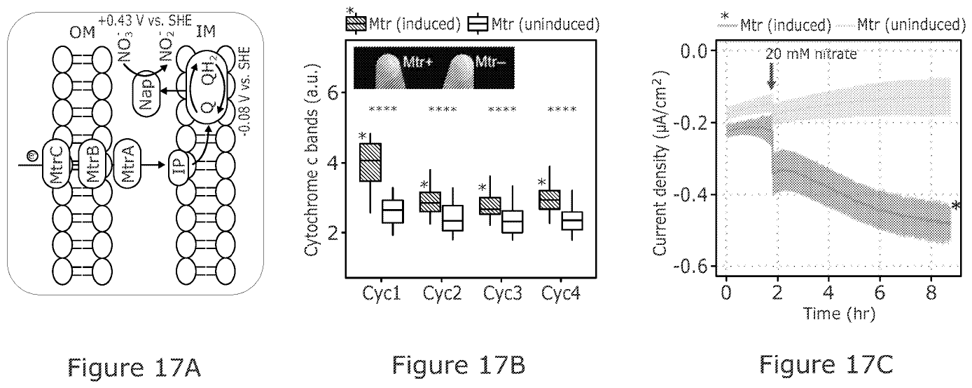

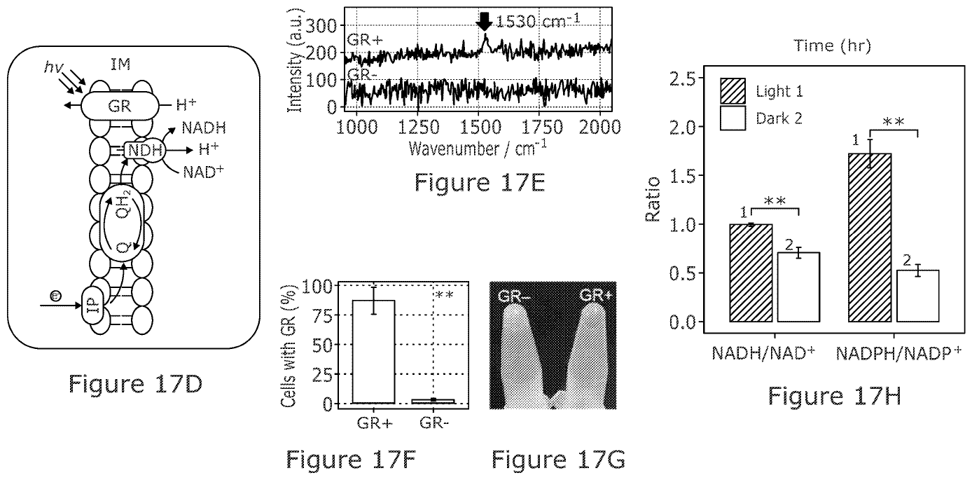

FIG. 17 Construction of a photosynthetic electron transport chain. (a) A MtrCAB complex mediates inward electron transfer to reduce nitrate. (b) Semi-quantification of c-type cytochrome levels in cells with or without Mtr complexes at the single-cell level by Raman analysis. Cy1, Cy2, Cy3 and Cy4 represent quantification of four bands at 748, 1128, 1312 and 1584 cm⁻¹. (c) Current consumption by induced and uninduced R. eutropha-Mtr with addition of 20-mM nitrate (d) Light-activated GR drives NADH dehydrogenase in reverse to synthesize NADH from quinone. (e) A typical Raman spectrum of a cell with GR complexes identified by a band at ˜1530 cm⁻¹ (red) and a typical Raman spectrum with the band intensity below background noise identified as a cell without GR (yellow). (f) The percentage of GR-expressing cells in induced and uninduced groups. (g) Uninduced and arabinose-induced cell pellets were yellow and pink, respectively. (h) NADH/NAD⁺ and NADPH/NADP⁺ ratios in the engineered cells under light and dark conditions. Statistics were performed with Student's t-test (**: p<0.01; ****: p<0.0001).

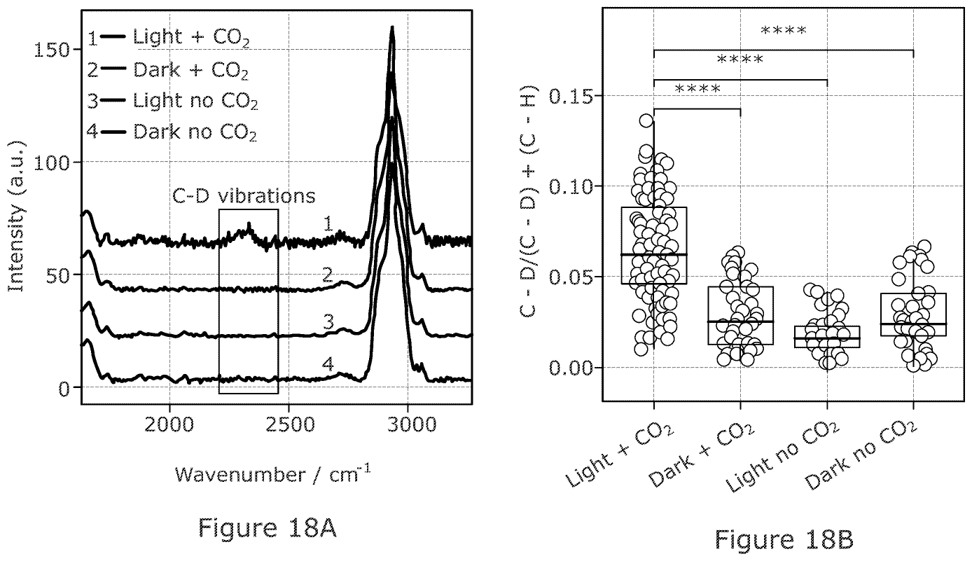

FIG. 18 Metabolic activities of R. eutropha-GR-Mtr probed by Raman C-D vibrations. (a) Averaged single-cell Raman spectra of R. eutropha-GR-Mtr cultured with 40% D20 under different conditions. The Raman band of C—H vibrations was shifted to a C-D band at 2070-2300 cm⁻¹, the extent of which represents metabolic activities of the cells. (b) Comparison of C-D/(C-D)+ (C—H) in the R. eutropha-GR-Mtr with and without light and CO₂ supply. Statistics were performed with Student's t-test (****: p<0.0001).

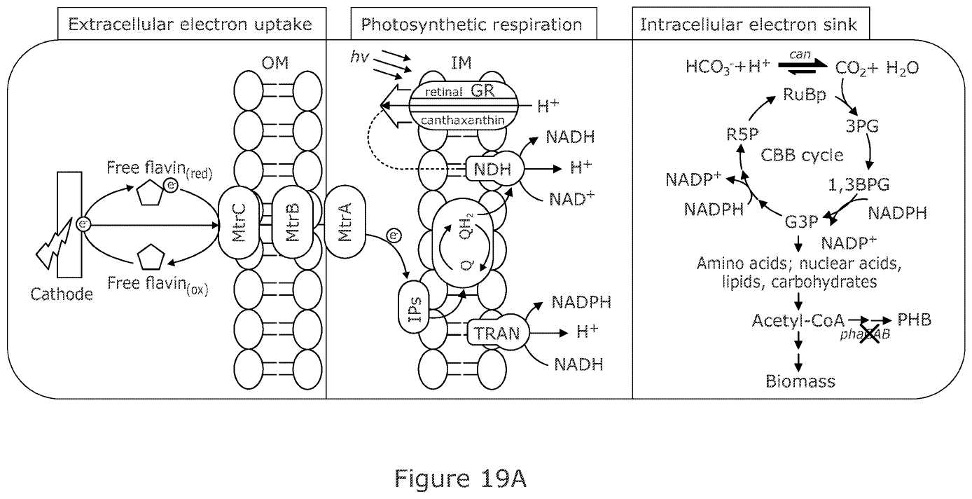

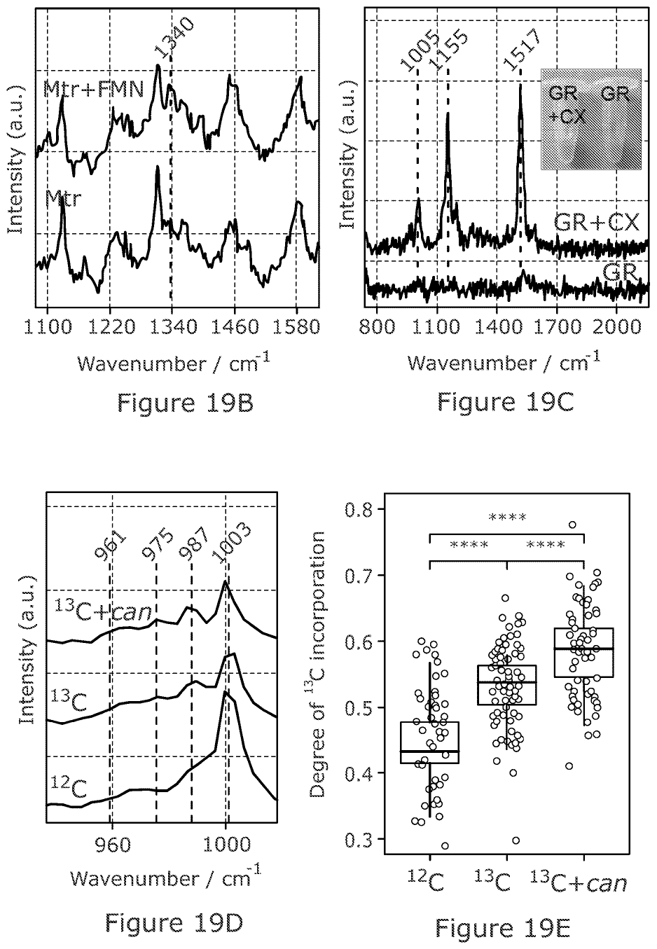

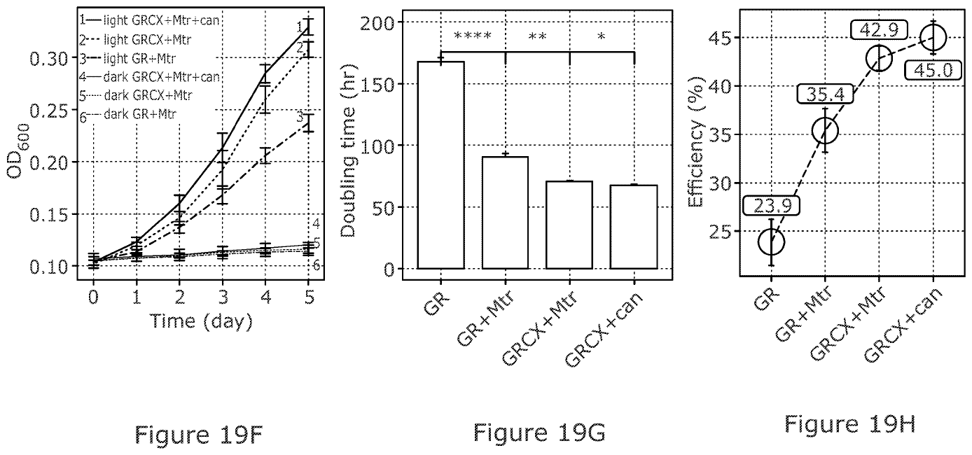

FIG. 19 Photoelectro-autotrophic growth of RMH5-GR-Mtr. (a) The photoelectrophic system can be divided into extracellular electron uptake, photosynthetic respiration and intracellular carbon sink. (b) A typical Raman spectrum of an RMH5-GR-Mtr cell with Mtr-bound FMN identified by a band at ˜1340 cm⁻¹. (c) A typical Raman spectrum of an RMH5-GR-Mtr cell with canthaxanthin (CX) identified by three bands at ˜1005, 1155 and 1517 cm⁻¹. (d) Cells grown in ¹³C-bicarbonate exhibited isotopic Raman shifts from 1003 cm⁻¹ to 987, 975, and 961 cm⁻¹ (e) Comparison of degree of ¹³C-bicarbonate incorporation of strains with and without can overexpression (f) Growth profiles of RMH5-GR-Mtr with and without canthaxanthin under light and dark conditions. (g) cell suspensions of induced RHM5-GR-Mtr with and without CX (f) Comparison of generation time of strains with GR, GR-Mtr, GRCX-Mtr-CX and GRCX-Mtr-can. (h) Comparison of faradaic efficiency of strains with GR, GR-Mtr, GRCX-Mtr-CX and GRCX-Mtr-can. Statistics were performed with Student's t-test (*: p<0.05; **: p<0.01; ***: p<0.001; ****: p<0.0001).

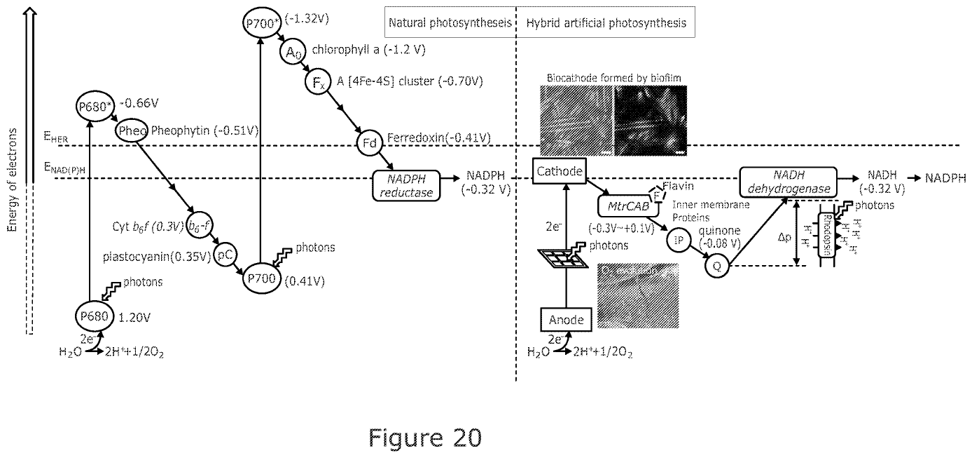

FIG. 20 Schematic illustration of the light reaction pathways of natural photosynthesis and designed artificial photosynthesis. Potentials for hydrogen evolution reaction (EHER) and NAD (P) H (ENAD (P) H) synthesis are highlighted by dashes lines. Part of redox enzymes are omitted. Oxygen evolution with O₂ bubbles was observed on the anode, and biofilms were observed on the cathode with brightfield microscopic image and FITC fluorescence image of biofilms attached to the cathode using SYTO™ 9 staining. The Scale bars represent 20 μm.

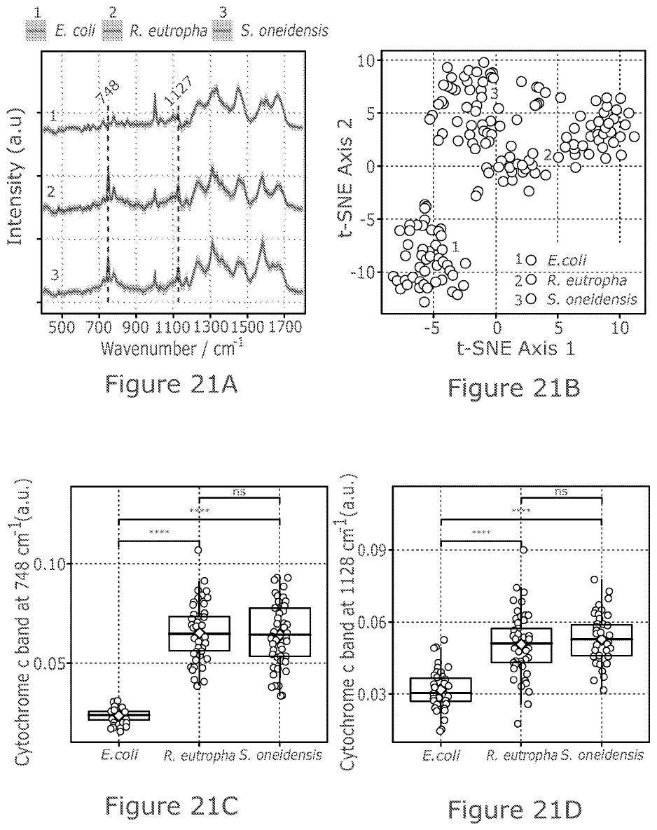

FIG. 21. Comparison of average Raman profiles of E. coli MG-1655, R. eutropha H16 and S. oneidensis MR-1. (a) Average single-cell Raman spectrum of E. coli MG-1655, R. eutropha H16 and S. oneidensis MR-1. (b) t-SNE visualization of sing-cell Raman spectrum showing clusters of different strains. (c-d) Semi-quantification of cytochromes levels by integrating Raman bands at (c) 748 and (d) 1128 cm⁻¹.

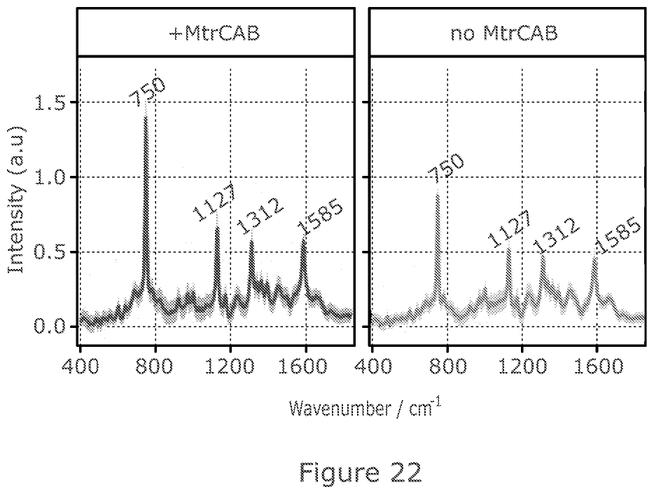

FIG. 22. Comparison of induced and uninduced R. eutropha-Mtr. Average single-cell Raman spectrum of induced and uninduced streains. Four Raman bands associated with cytochrome c are highlighted at 748, 1128, 1312, and 1585 cm⁻¹.

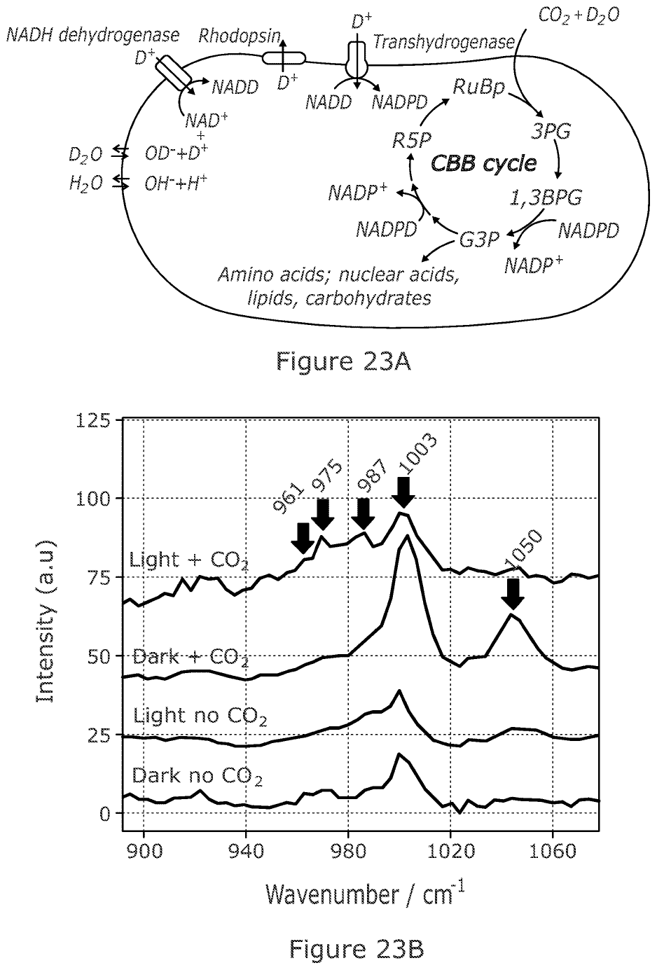

FIG. 23. (a) Schematic illustration of D20 involved in metabolisms based on CO₂ fixation. (b) Averaged single-cell Raman spectra of R. eutropha-Mtr-GR cultured with 40% D20 under different conditions. Raman shifts were observed from 1003 cm⁻¹ to 987, 975 and 961 cm⁻¹ in the cells incubated with illumination and CO₂. The Raman band associated with bicarbonate is highlighted at 1050 cm⁻¹.

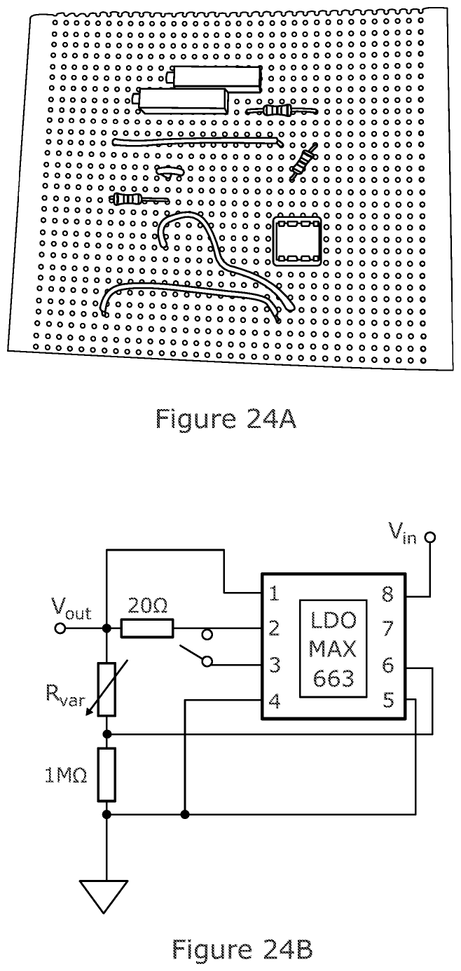

FIG. 24. Design of home-made voltage regulator. (a) A picture of the voltage regulator. (b) The circuit diagram of the voltage regulator.

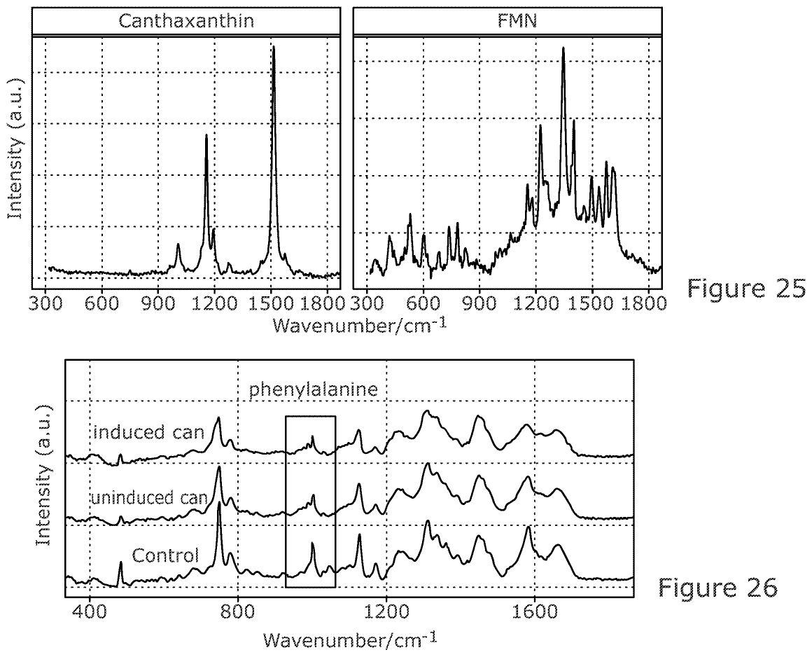

FIG. 25. Characterization of pure canthaxanthin and FMN by Raman analysis.

FIG. 26. Averaged single-cell Raman spectra of induced or uninduced RHM5-GR-can cultured with 20 mM ¹³C labbled bicarbonate, and phenylalanine is highlighted.

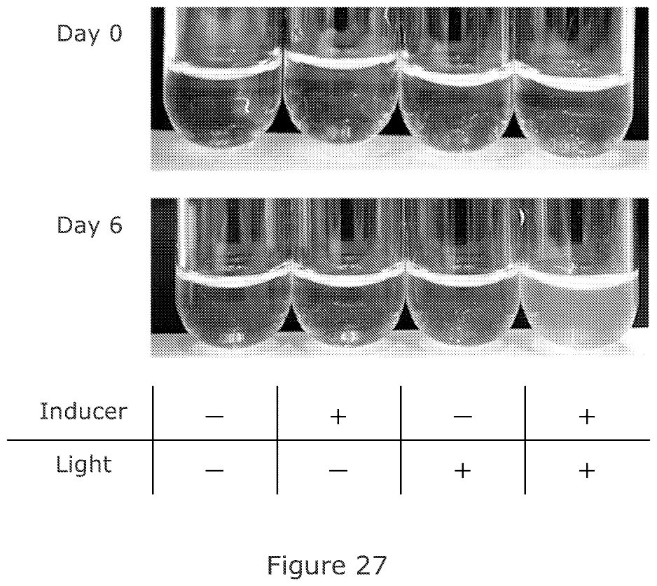

FIG. 27. Images of culture broth of the RHM5-Mtr-GR strain at day 0 and day 6 after with and without inducation under light and dark conditions.

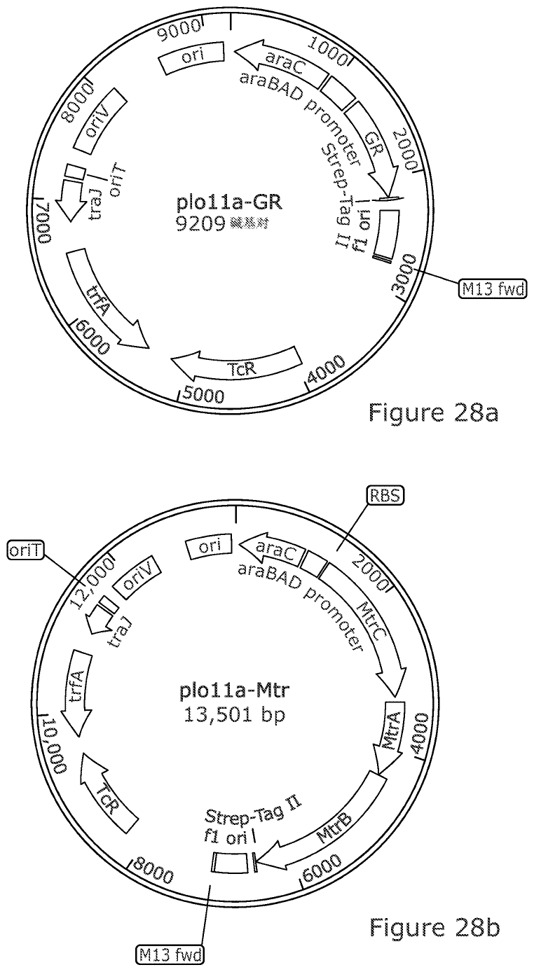

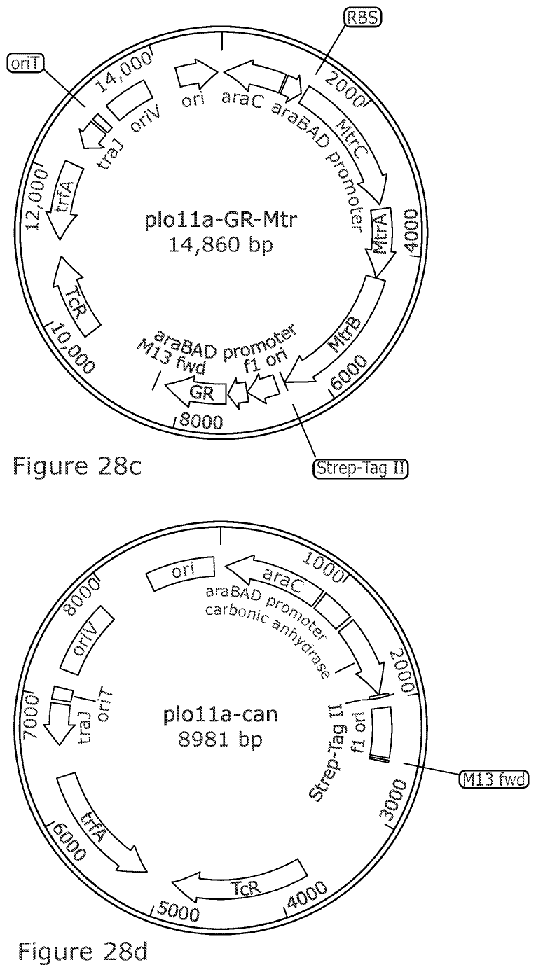

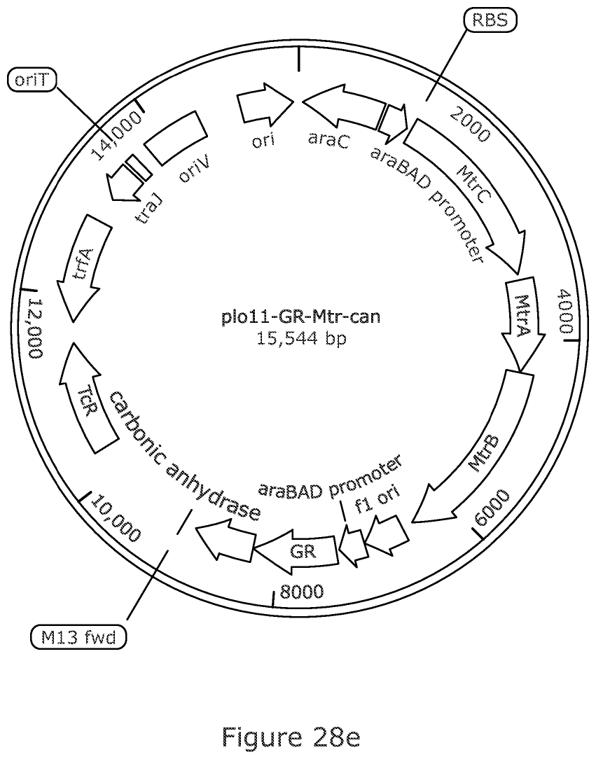

FIG. 28. Plasmid maps of (a) pLO11a-GR, (b) pLO11a-MtrCAB, (c) pLO11a-GRMtrCAB (d) pLO11a-can and (e) pLO11a-GR-Mtr-can.

### EXAMPLE 1—ENGINEERING A RHODOPSIN-BASED PHOTO-ELECTROSYNTHETIC SYSTEM IN BACTERIA FOR CO₂ FIXATION

### Summary

A key goal of synthetic biology is to engineer organisms that can use solar energy to convert CO₂ to biomass, chemicals and fuels. We engineered a light-dependent electron transfer chain by integrating rhodopsin and an electron donor to form a closed redox loop, which drives rhodopsin-dependent CO₂ fixation. A light-driven proton pump comprising Gloeobacter rhodopsin (GR) and its cofactor retinal have been assembled in Ralstonia eutropha (Cupriavidus necator) H₁₆. In the presence of light, this strain fixed inorganic carbon (or bicarbonate) leading to 20% growth enhancement, when formate was used as electron donor. We found that an electrode from a solar panel can replace organic compounds to serve as the electron donor, mediated by the electron shuttle molecule riboflavin. In this new autotrophic and photo-electrosynthetic system, GR is augmented by an external photocell for reductive CO₂ fixation. We demonstrated that this hybrid photo-electrosynthetic pathway can drive the engineered R. eutropha strain to grow using CO₂ as the sole carbon source. In this system, a bioreactor with only two inputs, light and CO₂, enables the R. eutropha strain to perform a rhodopsin dependent autotrophic growth. Light energy alone, supplied by a solar panel, can drive the conversion of CO₂ into biomass with a maximum energy efficiency of 4.1%, which is comparable with photosynthesis in plants.

### Significance

Rhodopsin and chlorophyll are two major light harvesting systems in nature, CO₂ fixation is mainly driven by chlorophyll-based photosynthesis. We demonstrate that a rhodopsin-based light-harvesting system can support CO₂ fixation in Ralstonia eutropha, which drives autotrophic growth using CO₂ as the sole carbon source, and with light as the only energy input. Such light energy alone, supplied by a solar panel, can drive the conversion of CO₂ into biomass with a maximum energy efficiency of 4.1%, comparable with chlorophyll-based photosynthesis. One implication of this work is that the elements of rhodopsin, recyclable electron mediator (e.g. riboflavin), light and electricity can be used to convert other bacteria, with native or engineered CO₂ fixation pathways, to photoautotrophic growth.

### INTRODUCTION

Chlorophyll-based photoautotrophic systems obtain electron donors from light-driven water splitting, and they also use light to generate a proton-motive force for ATP generation. Inspired by chlorophyll-based photoautotrophy (15), we hypothesised that a closed redox loop can be constructed by integrating rhodopsin with an electron donor. If the electron donor can be supplied by an electrode powered by solar panel, a rhodopsin-based photo-electrosynthetic system could drive autotrophic growth of bacteria using CO₂ as the sole carbon source, and with light as the only energy input.

To test this hypothesis, we engineered a light-powered electromicrobial system for CO₂ fixation (FIG. 1). The chosen bacterium, Ralstonia eutropha (Cupriavidus necator) H₁₆, lacks the capacity to use light as an energy source (16, 17), but its native CO₂ fixation pathway, is encoded on two operons of Calvin-Benson-Bassham (CBB) cycle, one on chromosome 2 and the other on its pHG1 megaplasmid (18). This bioenergetic system has only two inputs, light and CO₂. The bioenergy for R. eutropha H16 growth is supplied by installing Gloeobacter violaceus rhodopsin (GR), augmented by an external photocell that serves as the electron donor (FIG. 1). This engineered bacterium performs a hybrid form of photoautotrophy, which uses light to convert CO₂ into biomass.

### Results

Biosynthesis of β-Carotene in R. eutropha

Biosynthesis of carotenoids requires supply of the geranylgeranyl diphosphate (GGPP) precursor, followed by phytoene desaturation and subsequent modifications such as cyclisation or introduction of keto groups (19). The entire crtEXYIB operon for β-carotene synthesis, including the crtE promoter region (crt operon), from the pORANGE plasmid (20), was cloned into the pLO11 plasmid to make pCRT (Table 1 and FIG. 5A). The resulting production of β-carotene by R. eutropha H16 can be enhanced by overexpression of the dxr gene in pDxrCRT (Table 1 and FIG. 5B). An optimal pathway for-carotene synthesis in R. eutropha H16 is shown in FIG. 2A. Detailed information about the production of β-carotene in R. eutropha H16 (strain H16 pDxrCRT) is given in Supplementary Information and FIG. S2.

Biosynthesis of Gloeobacter Rhodopsins in R. eutropha

GR is a rhodopsin from the cyanobacterium Gloeobacter violaceus PCC7421 (Genbank: BAC88139) (21, 22). Cloning the plasmid pLO11-GR into R. eutropha H16 resulted in a distinct pink colour in the presence of the inducer arabinose and exogenous retinal (FIG. 2B). To examine the GR-retinal complex present in the intracellular membrane, a cell pellet from a 500 ml arabinose and retinal-induced culture of R. eutropha H16 containing pLO11-GR was disrupted using a French press and the membrane fraction purified on a sucrose density gradient. The fractionated cell extract from arabinose-induced cells generated a diffuse, highly coloured membrane band in the middle of the gradient. An absorbance spectrum (FIG. 2B) recorded on a sample harvested from the middle of this band revealed a peak at around 540 nm (arrowed) which is consistent with the published value for the GR-retinal complex (23).

Construction of a Gene Cluster for GR-Retinal Complex Biosynthesis in R. eutropha

Following the construction of two R. eutropha strains that separately produce β-carotene (H16 pDxrCRT) and GR (H16 GR), these new attributes were combined within a single strain to enable the assembly of a functional rhodopsin that confers the capacity for ATP synthesis in the light. The blh gene from the uncultured marine bacterium 66A03 encodes the β-carotene 15, 15′-dioxygenase enzyme (EC: 1.13.11.63), which cleaves one molecule of β-carotene into two molecules of retinal (FIG. 2A) in the presence of oxygen (24). This gene was codon optimised for R. eutropha and cloned into pDxrCRT to create pLO11-blhDxrCRT (Table 1), an expression vector for retinal production. The GR gene, under the control of a separate P_{BAD} promoter, was then inserted into this vector to create pLO11-blhDxrCRT-GR, for the production of GR holoprotein (FIG. 2C and Table 1). This construct was transformed into R. eutropha H16 to create R. eutropha H16 blhDxrCRT-GR (henceforth R. eutropha-GR), which acquired a pink colour in the presence of 0.1% arabinose inducer, indicating assembly of a GR-retinal holocomplex. Solvent extraction of the pellets followed by HPLC analysis showed that the uninduced sample contained no β-carotene (FIG. 7A, broken line) or retinal (FIG. 7B, broken line) but upon induction a small amount of β-carotene (FIG. 7A, solid line) and a larger amount of retinal, appearing as an elution peak at approximately 8 minutes under these running conditions, could be detected (FIG. 7B, solid line). This peak had an absorbance spectrum identical to that seen for all trans-retinal with an absorbance maximum at 380 nm (FIG. 7C, solid line). Following induction two further peaks appeared at 8.9 and 9.2 minutes (FIG. 7B labelled 1 and 2, respectively) with absorbance spectra (FIG. 7C, broken lines) similar to all trans-retinal but with shifted absorbance maxima of 386 nm (peak 1) and 395 nm (peak 2), likely to be isomers of trans-retinal. Raman micro-spectroscopy was used to examine GR-retinal complexes in R. eutropha H16 and E. coli JM109. Single-cell Raman spectra (SCRS) of cells synthesising GR display a unique band at 1530 cm⁻¹ (FIG. 8), which is a characteristic biomarker for the retinal-GR holoenzyme, similar to that previously reported for the retinal-PR holoenzyme (25).

GR Expression in R. eutropha H16 Significantly Enhances Cell Growth

Initially formate was used as the organic electron donor to investigate the light-dependent growth of the engineered R. eutropha-GR strain. FIG. 3A shows that when arabinose was added as the inducer under micro-aerobic conditions illumination by white LED light enhanced the biomass of the R. eutropha-GR strain by up to 24%, relative to either uninduced or dark-grown cells. The growth rate of R. eutropha-GR has increased 33% in the light, compared that in the dark. Light-enhanced growth was maintained over the five-day time-course of the experiment, indicating a significant light-dependent increase in biomass of the R. eutropha-GR strain. Control strains grown in the presence of arabinose alone (no formate) did not show any growth (FIG. 3A), ruling out the possibility that the inducer can be used as a carbon source. R. eutropha H16 with no GR expression (containing pLO11-blhDxrCRT-GR but no arabinose induction) showed no detectable growth difference between light and dark conditions (FIG. 3A). Thus, this is evidence that holo-GR produced in R. eutropha-GR can capture light energy to increase cell growth in the presence of formate.

The light energy harvested by GR significantly enhanced PHB accumulation in R. eutropha-GR when formate was used as the sole carbon source (FIG. 9). This also confirms that energy from light-driven proton pumping by GR stimulates biosynthesis Raman micro-spectroscopy was employed to examine cells containing GR. After growth of R. eutropha-GR in minimal medium (MM) with formate in the dark or light for 3 days, the position of the 1530 cm⁻¹ Raman band, characteristic for GR, remained the same (FIG. 10). We further measured SCRS of the bacterial population (200 or 400 single cells) and calculated the percentage of the sub-population expressing GR, i.e. with the 1530 cm⁻¹ GR band (Table 4 and FIG. 3B). At the outset 83% of the R. eutropha H16 population synthesised holo-GR (FIG. 3B); this proportion declined only to 69% by day 3 under micro-aerobic conditions in the light, in the absence of antibiotic selection pressure, whilst it dropped to 37% in the dark (FIG. 3B). This indicates that R. eutropha —GR in the light had a growth advantage over the cells without GR, leading to an enrichment of those cells with GR.

CO₂ Fixation by GR-Expressing R. eutropha H16

Stable isotope probing was used to examine CO₂ fixation by the R. eutropha-GR strain. Formate (40 mM) or bicarbonate (40 mM) were used under three differing conditions: (i) ¹²C-formate+¹²C-bicarbonate; (ii) ¹³C-formate+¹²C-bicarbonate; and (iii) ¹²C-formate+¹³C-bicarbonate. Under these three labelling conditions illumination for 3 days improved growth by 18% (FIG. 11A), 20% (FIG. 11B) and 20% (FIG. 11C), respectively. Incorporation of a ¹³C-substrate was detected via shifted Raman bands in SCRS (26), as shown in FIG. 3C for R. eutropha-GR grown in ¹³C-formate and ¹³C-bicarbonate, compared to 12C substrates. In particular, two Raman bands corresponding to cytochrome c (27) at 748 (pyrrole breathing mode) and 1127 cm⁻¹ (CN stretching vibrations), and a band arising from proteins at 1655 cm⁻¹ (Amide I), are shifted to 732, 1115, and 1619 cm⁻¹, respectively (FIG. 3C). The single band at 1003 cm⁻¹ is characteristic of the phenyl ring of ¹²C-phenylalanine becomes three bands at 987, 975 and 961 cm⁻¹ in the ¹³C amino acid (FIG. 3C), in good agreement with the ¹³C-Raman shifts observed in previous work (28). Isotope labelling with ¹³C-bicarbonate (FIG. 3C) confirms CO₂ fixation by R. eutropha-GR, and subsequent incorporation into biomass.

We further sought to compare the extent of ¹³C incorporation from ¹³C-bicarbonate and ¹³C-formate in the light (FIG. 12). The Raman band for pyrrole ring breathing in cytochrome c, originally at 748 cm⁻¹ (FIG. 12), exhibited a larger downshift to 732 cm⁻¹ in cells grown with ¹³C-bicarbonate, relative to the 737 cm⁻¹ observed with ¹³C-formate (FIG. 12). We also quantified the newly synthesised proteins in single cells by calculating the area ratio of the 1655 cm⁻¹ to 1619 cm⁻¹ bands and found higher amounts of these proteins with ¹³C-bicarbonate relative to ¹³C-formate (p<0.0001) (FIG. 12). These results suggest that R. eutropha-GR could also fix CO₂ with formate acting as an organic electron donor. When formate acts as the carbon source it is converted into CO₂ and NADH by R. eutropha H16 (4), with the CO₂ being fixed via the Calvin-Benson-Bassham (CBB) cycle and the NADH transformed into NADPH, via a proton-translocating transhydrogenase, to support cell growth (18, 29). R. eutropha H16 may use the CO₂ from both degraded formate and the medium without differentiation. When extra energy is available via the light harvesting GR, R. eutropha-GR fixed more CO₂ from the medium.

Sources of Reductant for Light-Driven Autotrophic Growth of R. eutropha

A hybrid form of photoautotrophic system was established to grow R. eutropha-GR, (FIG. 13) using CO₂ and light as inputs, and biomass generation as the output. The light source serves two purposes: driving the solar panel to produce electricity, and activating intracellular GR in the cathode chamber to generate ATP (FIG. 13).

Rather than using formate, a solid-phase electrode can also be directly used as the electron donor for CO₂ fixation which requires an electron shuttle to transfer electrons from the cathode to the cells. We used neutral red- and riboflavin-mediated electron transfer from an electrode in a bioreactor with CO₂ as the sole source of carbon. Neutral red (NR) has been used to accept electrons from the cathode and deliver them into microorganisms to increase the level of intracellular reducing equivalent (e.g., NADH), because it has a low standard reduction potential (−525 mV vs Ag/AgCl) similar to that of NADH/NAD⁺ (−520 mV vs Ag/AgCl) (30). The generated NADH can be converted to NADPH by transhydrogenase for carbon fixation. A significant increase in cell number was observed in the presence of light and NR, indicating the potential of the electron shuttle-mediated system in carbon fixation (FIG. 14). However, NR is not recyclable as it enters cells for redox reactions and remains inside bacterial cells after transferring electrons (FIG. 14B). Hence, NR is not a reusable and sustainable electron shuttling molecule, unfavourable for the long-term operation of the system.

Riboflavin (−400 mV vs Ag/AgCl) can also serve as an electron shuttling molecule. It interacts with outer membrane-bound cytochromes and can transfer electrons from bacteria to an electrode (31), but the reverse process is thermodynamically unfavourable (30). Interestingly we found that riboflavin is able to transfer electrons from the cathode to bacterial cells when a light-driven proton pump (such as the GR) is present to overcome the energy hurdle. FIG. 4A illustrates the electron transport chain for the reductive fixation of CO₂, which operates by reversing electron flow from the electron transfer chain to the quinol pool and onto NADH dehydrogenase. In this electron transport chain, a proton-translocating transhydrogenase is required, which couples the reduction of NADP⁺by NADH to the inward translocation of protons across the cytoplasmic membrane (29, 32). Thus, the transmembrane proton gradient created by the light-driven turnovers of GR enables electron shuttling via riboflavin, leading to NADPH formation and ATP production by the ATP synthase (FIG. 4A).

The reversible redox reactions of riboflavin were used to shuttle electrons from the electrode to the cell in the bioreactor because R. eutropha is non-exoelectrogenic. We used cyclic voltammetry (CV) to verify these redox reactions at the electrode, which was made from carbon cloth. The cyclic voltammogram in FIG. 4B shows typical oxidative and reductive peaks, indicating the suitability of the electrode material, and yielding a midpoint potential for riboflavin of −0.39 V versus a Ag/AgCl standard electrode (FIG. 4B). It also shows that when carbon cloth is used as the cathode electrode without riboflavin could not transfer electrons. A colour change in the medium indicated that some of the riboflavin was oxidised, consistent with electron transfer between riboflavin and R. eutropha H16 (FIG. 4B).

We used the bioreactor to demonstrate that R. eutropha-GR could perform photoautotrophic growth in the presence of the −0.6 V cathode, driven by light-activated GR and in the absence of formate (FIG. 4C). Several negative controls show there was no detectable growth in the dark, even though the electrode potential, riboflavin and GR were available, nor in uninduced cells lacking GR but with riboflavin and the electrode potential (FIG. 4C), nor in the absence of riboflavin (FIG. 4C).

Autotrophic growth of R. eutropha-GR in the bioreactor was assessed using cell counts (FIG. 4C) and verified by isotope labelling with ¹³CO₂ as the sole carbon source for 4 days (FIG. 4D). SCRS was used to analyse 200 single cells of R. eutropha-GR, which revealed multiple Raman shifts arising from integration of ¹³CO₂ into cellular biomass. Raman bands corresponding to cytochrome c at 748 (pyrrole breathing mode) and 1127 cm⁻¹ (CN stretching vibrations), and a band arising from proteins at 1655 cm⁻¹ (Amide I), shifted to 728, 1113, and 1623 cm⁻¹, respectively (FIG. 4D). SCRS was also used to quantify the number of GR-containing bacterial cells from the bioreactor (FIG. 8), which showed no significant change after 4 days (FIG. 4E), and reflecting a likely selective advantage conferred by GR, despite the absence of antibiotics.

The overall voltage to drive photo-electrosynthetic growth of R. eutropha-GR in the bioreactor was 1.6-1.8 V. The corresponding light intensity on the solar panel was only 2 μmol m⁻² s⁻² (FIG. 15), suggesting the bioreactor could operate in weak daylight. During the exponential growth phase (day 3-4 in FIG. 4C), the maximal energy conversion efficiency from electricity to biomass was 20%, and the efficiency from solar panel to biomass was 4.1%. This overall efficiency is comparable to the number for plants (33).

Collectively, the results suggest that R. eutropha-GR can utilise light as an energy source to drive the CO₂ fixation pathway for cell growth, in effect converting R. eutropha from chemolithoautotrophy to a new growth mode that is a hybrid form of photoautotrophy that could be termed photoelectroautrophy.

### DISCUSSION

The results of this study confirm that R. eutropha-GR can be engineered to grow autotrophically, using light to supply energy for GR to pumping protons and for riboflavin to mediate electron transfers (FIG. 4C). R. eutropha has been engineered to perform a type of hybrid photosynthesis, in which GR acts as a light-driven proton pump. The resulting proton motive force drives the transhydrogenase that forms NADPH, used for reductive fixation of CO₂, as well as driving ATP synthase to produce ATP. Electrons required for the reductive assimilation of CO₂ using the native Calvin-Benson-Bassham (CBB) cycle can be provided by either an electrode (mediated by riboflavin) or an organic compound such as formate (FIG. 1). Accordingly, biomass production in the light can be enhanced by 20% when formate is used as the electron donor (FIG. 3A), but biomass can also be produced in a bioreactor with a CO₂ carbon supply and a light source as the only inputs (FIG. 4C). The biomass of R. eutropha H16 is a good source of single cell protein for animal feed due to its high protein content, and it has been awarded a qualified presumption of safety (QPS) in EU (16, 17).

The conversion of R. eutropha H16 from chemolithoautotrophy to a hybrid form of photoautotrophy has required the introduction of β-carotene biosynthesis, via the insertion of a four gene biosynthetic pathway and overexpression of the dxr gene, and its subsequent conversion into retinal by adding a gene encoding β-carotene 15 15′-dioxygenase. The introduction of the gene encoding the GR creates a photosynthetic system built from a single rhodopsin protein, compared to one that relies on a series of chlorophyll protein complexes. As GR has a pKa=˜4.8 (34), compared to PR which has a pKa=˜7.5 (35), GR is functional at a lower pH, a situation often found in R. eutropha H16 growing using formate or CO₂ as the sole carbon source. It has been reported that GR expression in E. coli has improved phototrophic metabolism (36) and GR has a higher molecular proton pumping rate than PR (37). GR is originally from thylakoid-less Gloeobacter violaceus PCC7421 (38), its high efficiency of proton pumping and rapid photocycle is probably a compensation to the shortage of energy generation from chlorophyll-based photosynthesis (22).

It has been shown that Ralsonia eutropha H16 was able to produce alcohols from electrochemically generated formate (4). The challenge of this approach is to overcome the toxic metals from dissolved electrodes during the electrochemical process. Rhodopseudomonas palustris can perform] photoautotrophic CO₂ fixation by extracellular electron uptake (EEU) from solid-phase conductive substances (39, 40). This photoautotrophic activity in Rhodopseudomonas palustris is chlorophyll-based (41).

In this study, we demonstrated that we can design light-dependent electron transfer chain to drive photo-electrosynthesis using rhodopsin-based system. Such system only requires a rhodopsin and an electron mediator such as riboflavin. Microbial rhodopsin is a simple light-driven proton pump found broadly distributed in nature (8, 9), and it can also be easily engineered into different hosts of bacteria (8, 12, 13). Furthermore, GR has been shown to combine with other retinal analogues to absorb near-infrared light (850-950 nm) (42), which can significantly extend the light harvesting spectrum and maximise energy harvesting per surface area (43). The recyclable electron mediator-riboflavin can be readily synthesised by bacteria (44) or manually added into the reactor. Hence, the application of such a rhodopsin-based light harvesting system would enable the conversion of various naturally heterotrophic bacteria into photoautotrophs that are able to use light for CO₂ fixation. In the natural oxygenic photosynthesis, a chlorophyll-based photosystem splits water and provides electrons to the redox reaction. The hybrid photoelectroautrophic system shown here mimics photosynthesis using GR to generate a proton gradient and electricity as the electron donor. The result is that a new mode of photosynthetic growth has been engineered, enabling R. eutropha H16 to use solar energy to convert CO₂ into biomass.

### Materials and Methods

### Bacterial Strains and Culture Conditions

E. coli strains were grown in LB broth at 37° C. under aeration by shaking at 200 rpm. R. eutropha strains were grown in LB broth at 30° C. under aeration by shaking at 150 rpm. If required, antibiotics (Sigma-Aldrich) were added as follows: 10 μg ml⁻¹ gentamicin, 10 μg ml⁻¹ tetracycline, 400 μg ml⁻¹ kanamycin and 500 μg ml⁻¹ ampicillin for R. eutropha; 12.5 μg ml⁻¹ tetracycline and 50 μg ml⁻¹ kanamycin for E. coli. Induction of strains transformed with the pLO11 expression vector containing the arabinose-inducible P_{BAD} promoter was carried out by growth to log phase and the addition of 0.1% (w/v) L-arabinose (Sigma-Aldrich) and overnight growth at 30° C. for R. eutropha and 0.2% (w/v) L-arabinose and overnight growth at 37° C. for E. coli. Where required, induction of proteorhodopsin (PR) expression was accompanied by the addition of exogenous trans-retinal (Sigma-Aldrich) to a final concentration of 5 μg ml⁻¹.

All constructs were assembled and expressed in a commercially obtained, chemically competent E. coli JM109 strain (Promega, UK). Two gentamicin-resistant R. eutropha strains, namely H16 (ATCC 17699) and its derivative strain RHM5 (gift from Min-Kyu Oh, Korea University, South Korea; hereafter H16Δpha) in which the phaCAB operon encoding the genes required for the conversion of acetyl-CoA to polyhydroxybutyrate (PHB) have been deleted (45), were used for expression of constructs. Recombinant plasmids were transformed into the E. coli S17-1 strain (46), made chemically competent using standard techniques (47), for transfer into R. eutropha by conjugative plasmid transfer using biparental mating.

### Plasmid Construction

Common cloning procedures were performed according to standard protocols (47). Polymerase chain reaction (PCR) was carried out using Q5 DNA polymerase (NEB, UK) and synthesised primers (Sigma-Aldrich) according to the manufacturer's instructions, and all PCR products were checked by DNA sequencing (Eurofins, Germany). A list of the plasmids and primers used in this study are shown in Table 1 and Supplementary Table 2 respectively and details of plasmid construction are given in Supporting Information.

### Single-Cell Raman Spectra (SCRS) Measurements and Analysis

Bacterial cells were washed three times with distilled water to remove traces of culture medium prior to measurements. Samples were diluted until individual bacterial cells could be observed under a 100×/0.75 microscope and a 1.5 μl suspension was dropped onto an aluminium-coated slide and air dried. SCRS were obtained using a 532-nm neodymium-yttrium aluminium garnet laser with a 300 grooves mm⁻¹ diffraction grating (LabRAM HR Evolution, HORIBA, UK) and were acquired in the range of 100-3200 cm⁻¹. The laser power was set at ˜30 mW which was attenuated by neutral density (ND) filters before focusing onto the samples. For the measurements of the Gloeobacter violaceus PCC7421 rhodopsin (GR) complexes, 1% power filter and 1-second acquisition time were used; the low power and short acquisition time were used to prevent photobleaching of the chromophores. Each condition was measured with two biological replicates; each replicate was measured with more than 150 and 100 single cells in induced and uninduced samples, respectively, or 200 and 400 single cells before and after growth, respectively, under aerobic and microaerobic conditions. Cells with GR complexes were identified by a band at ˜1530 cm⁻¹. As in the measurements for determining utilisation of formate, bicarbonate and carbon dioxide, 25% power filter and 3 to 5-second acquisition time were used to acquire spectra with high signal-to-noise ratios. Each condition was measured with two to three biological replicates, each with more than 30 to 50 single cells acquired. Spectra were recorded with LABSPEC 6 software (HORIBA, UK). All raw spectra were pre-processed by cosmic ray correction, polyline baseline fitting and subtraction and vector normalisation of the entire spectral region. Quantification of biomolecules was done by integrating the area of the corresponding Raman bands. All analysis and plotting were done under a R 4.0.0 environment.

Growth of R. eutropha Strains Under Light and Dark

Growth characterization experiments of R. eutropha with pLO11-blhDxrCRT-GR were conducted under light and dark conditions, with and without induction, respectively. Before the experiments, R. eutropha strains harbouring the plasmid were pre-cultivated overnight in tryptic soy broth (TSB) medium (with 10 μg/ml of tetracycline, 5 μg/ml of trans-retinal and 0.2% (w/v) L-arabinose) at 30° C. under aeration by shaking at 150 rpm. After TSB preculture, cells were harvested by centrifugation at 3000 g for 5 min. The supernatant was discarded, and cells were washed three times with a minimal medium (MM: 6.74 g/L Na₂HPO4·7H₂O, 1.5 g/L KH₂PO₄, 1.0 g/L (NH₄)₂SO₄, 1 mg/L CaSO₄: 2H₂O, 80 mg/L MgSO₄·7H₂O, 0.56 mg/L NiSO₄·7H₂O, 0.4 mg/L ferric citrate, 200 mg/L NaHCO₃, and pH 7.0). Cells were grown under eight different conditions: MM with and without 80 mM formate in normal aerobic and micro-aerobic environments; MM containing 0.2% (w/v) L-arabinose as the inducer, with and without formate in micro-aerobic environment which were created in 15 mL tubes filled with 12 mL medium. Neither antibiotics nor exogenous trans-retinal was added in any of these conditions. Each growth condition was illuminated with a white LED light (˜50 μmol/s/m²) and dark wrapped in foil. In total, 6 growth conditions were analysed with each condition having three replicates. Cells were initially resuspended in the MM to a final optical density (OD) of ˜0.01 and grown at 30° C. with shaking at 150 rpm.

Bicarbonate Utilisation in R. eutropha Growth

R. eutropha with pLO11-blhDxrCRT-GR was grown in MM containing 0.2% (w/v) L-arabinose micro-aerobically, using 40-mM formate and 40-mM bicarbonate as carbon sources. In order to investigate inorganic carbon fixation and the impact of bicarbonate utilisation on biomass synthesis, ¹³C isotope-labelled formate and bicarbonate were used for growth experiments under the following isotopic conditions: (i) ¹²C-formate+¹²C-bicarbonate; (ii) ¹³C-formate+¹²C-bicarbonate; and (iii) ¹²C-formate+¹³C-bicarbonate. Each experiment was carried out under light or dark conditions, and no antibiotics were added. Single cell Raman spectroscopic measurements were carried out and SCRS were used for the isotopic analysis.

### Microbial Photoelectrochemical System Apparatus

A dual-chamber bioreactor (70 mL for each chamber) separated by a Nafion membrane (only allowing proton transfer) was used as a bio-photoelectrochemical system for microbial growth experiments. For the anode chamber, carbon cloth (H23, 95 g m⁻²; 2.5× 4.0 cm²; Quintech, Gloucestershire, UK) with a platinum catalyst (1 mg cm 2, PtC 60%; FuelCellStore) was used as the counter electrode. For the cathode chamber, the working electrode was made of 3.0×3.0 cm² carbon cloth and a Ag/AgCl reference electrode (3M KCl, RE-5B, BASi, USA) was installed for measuring the potentials. R. eutropha with pLO11-blhDxrCRT-GR was precultured in TSB and pretreated as above with the minimal medium to give an initial OD₆₀₀ of 0.01 before being injected into the cathode chamber. An electron shuttle (50 μM riboflavin or 50 μM neutral red) was then added to the chamber as the electron mediator. A polycrystalline solar panel (1.5 W, 140 mm×180 mm, RS, UK) was used to generate electricity powered by light. Photovoltaic characterisation of the solar panel is shown in FIG. 15. The potential of the working electrode was controlled by a designed potentiostat. In the experiments, the potential was kept at −0.6 V [versus Ag/AgCl]. The cathode chamber was bubbled with CO₂ at a flow rate of 20 mL/min, illuminated with white LED light (˜50 μmol/s/m²), operated at 30° C., and agitated at 150 rpm. Pictures of microbial photoelectrochemical system apparatus can be found in FIG. 14.

The electricity transfer efficiency of the biomass formation could be calculated by the equation

$$η = \frac{n z F}{\int i dt}$$

Where n is the amount of biomass (using classic formula C5H7NO2) product (mol), z is the number of transferred electrons (z=4 for CO₂ conversion to biomass), F is the Faraday constant (96,485 C/mol), i is the current (A), and t is the time(s). In the solar panel system, the average electricity current is ˜11.2 μA under stable operation.

### REFERENCES

1. J. M. Clomburg, A. M. Crumbley, R. Gonzalez, Industrial biomanufacturing: The future of chemical production. Science 355, eaag0804 (2017).
2. P. Durre, B. J. Eikmanns, C1-carbon sources for chemical and fuel production by microbial gas fermentation. Curr Opin Biotech 35, 63-72 (2015).
3. S. Gleizer et al., Conversion of Escherichia coli to Generate All Biomass Carbon from CO₂. Cell 179, 1255-1263 (2019).
4 H. Li et al., Integrated Electromicrobial Conversion of CO₂ to Higher Alcohols. Science 335, 1596-1596 (2012).
5. J. Panich, B. Fong, S. W. Singer, Metabolic Engineering of Cupriavidus necator H16 for Sustainable Biofuels from CO₂. Trends in Biotechnology 39, 412-424 (2021).
6. C. Liu, B. C. Colon, M. Ziesack, P. A. Silver, D. G. Nocera, Water splitting-biosynthetic system with CO₂ reduction efficiencies exceeding photosynthesis. Science 352, 1210-1213 (2016).
7. D. A. Bryant, N. U. Frigaard, Prokaryotic photosynthesis and phototrophy illuminated. Trends Microbiol 14, 488-496 (2006).
8. O. M. Finkel, O. Beja, S. Belkin, Global abundance of microbial rhodopsins. Isme J 7, 448-451 (2013).
9. L. Gomez-Consarnau et al., Microbial rhodopsins are major contributors to the solar energy captured in the sea. Sci Adv 5, aaw8855 (2019).
10. D. L. Kirchman, T. E. Hanson, Bioenergetics of photoheterotrophic bacteria in 10. the oceans. Env Microbiol Rep 5, 188-199 (2013).
11. E. F. DeLong, O. Beja, The Light-Driven Proton Pump Proteorhodopsin Enhances Bacterial Survival during Tough Times. Plos Biol 8, e1000359 (2010).
12. O. Beja et al., Bacterial rhodopsin: Evidence for a new type of phototrophy in the sea. Science 289, 1902-1906 (2000).
13. O. Beja, E. N. Spudich, J. L. Spudich, M. Leclerc, E. F. DeLong, Proteorhodopsin phototrophy in the ocean. Nature 411, 786-789 (2001).
14. G. P. Hu et al., Light-driven CO2 sequestration in Escherichia coli to achieve theoretical yield of chemicals. Nature Catalysis 4, 395-406 (2021).
15. D. R. Ort et al., Redesigning photosynthesis to sustainably meet global food and bioenergy demand. Proceedings of the National Academy of Sciences of the United States of America 112, 8529-8536 (2015).
16. J. Sillman et al., Bacterial protein for food and feed generated via renewable energy and direct air capture of CO₂: Can it reduce land and water use? Global Food Security-Agriculture Policy Economics and Environment 22, 25-32 (2019).
17. K. Koutsoumanis et al., Update of the list of QPS-recommended biological agents intentionally added to food or feed as notified to EFSA 11: suitability of taxonomic units notified to EFSA until September 2019. Efsa Journal 18 (2020).
18. A. Pohlmann et al., Genome sequence of the bioplastic-producing “Knallgas” bacterium Ralstonia eutropha H16. Nature Biotechnology 24, 1257-1262 (2006).
19. A. R. Moise, S. Al-Babili, E. T. Wurtzel, Mechanistic Aspects of Carotenoid Biosynthesis. Chemical reviews. 114, 164-193 (2014).
20. S. Y. Kim, S. A. Waschuk, L. S. Brown, K. H. Jung, Screening and characterization of proteorhodopsin color-tuning mutations in Escherichia coli with endogenous retinal synthesis. Biochim Biophys Acta 1777, 504-513 (2008).
21. Y. Nakamura et al., Complete genome structure of Gloeobacter violaceus PCC 7421, a cyanobacterium that lacks thylakoids. DNA Res 10, 137-145 (2003).
22. A. R. Choi, L. C. Shi, L. S. Brown, K. H. Jung, Cyanobacterial Light-Driven Proton Pump, Gloeobacter Rhodopsin: Complementarity between Rhodopsin-Based Energy Production and Photosynthesis. Plos One 9 (2014).
23. M. K. M. Engqvist et al., Directed Evolution of Gloeobacter violaceus Rhodopsin Spectral Properties. J Mol Biol 427, 205-220 (2015).
24. Y. S. Kim, C. S. Park, D. K. Oh, Retinal production from beta-carotene by beta-carotene 15,15′-dioxygenase from an unculturable marine bacterium. Biotechnol Lett 32, 957-961 (2010).
25. Y. Z. Song et al., Proteorhodopsin Overproduction Enhances the Long-Term Viability of Escherichia coli. Applied and Environmental Microbiology 86, e02087-02019 (2020).
26. Y. Wang, W. E. Huang, L. Cui, M. Wagner, Single cell stable isotope probing in microbiology using Raman microspectroscopy. Curr Opin Biotech 41, 34-42 (2016).
27. J. B. Xu, I. Webb, P. Poole, W. E. Huang, Label-Free Discrimination of Rhizobial Bacteroids and Mutants by Single-Cell Raman Microspectroscopy. Analytical Chemistry 89, 6336-6340 (2017).
28. J. B. Xu et al., Raman Deuterium Isotope Probing Reveals Microbial Metabolism at the Single-Cell Level. Analytical Chemistry 89, 13305-13312 (2017).
29. M. Raberg et al., Proteomic and Transcriptomic Elucidation of the Mutant Ralstonia eutropha G (+)₁ with Regard to Glucose Utilization. Applied and Environmental Microbiology 77, 2058-2070 (2011).
30. X. Chen, Y. Cao, F. Li, Y. Tian, H. Song, Enzyme-Assisted Microbial Electrosynthesis of Poly(3-hydroxybutyrate) via CO₂Bioreduction by EngineeredRalstonia eutropha. ACS Catalysis 8, 4429-4437 (2018).
31. C. K. Ng et al., Genetic engineering biofilms in situ using ultrasound-mediated DNA delivery. Microbial Biotechnology 14, 1580-1593 (2021).
32. J. B. Jackson, A review of the binding-change mechanism for proton-translocating transhydrogenase. Biochimica et Biophysica Acta (BBA)—Bioenergetics 1817, 1839-1846 (2012).
33. X.-G. Zhu, S. P. Long, D. R. Ort, What is the maximum efficiency with which photosynthesis can convert solar energy into biomass? Curr Opin Biotech 19, 153-159 (2008).
34. T. Tsukamoto et al., Salt bridge in the conserved His-Asp cluster in Gloeobacter rhodopsin contributes to trimer formation. Febs Letters 587, 322-327 (2013).
35. S. Hirschi, D. Kalbermatter, Z. Ucurum, T. Lemmin, D. Fotiadis, Cryo-EM structure and dynamics of the green-light absorbing proteorhodopsin. Nature Communications 12 (2021).
36. H. A. Kim et al., An evolutionary optimization of a rhodopsin-based phototrophic metabolism in Escherichia coli. Microbial Cell Factories 16 (2017).
37. Q. Chen, J. Arents, S. Ganapathy, W. J. de Grip, K. J. Hellingwerf, Functional Expression of Gloeobacter Rhodopsin in Synechocystis sp PCC6803. Photochemistry and Photobiology 93, 772-781 (2017).
38. J. Mares et al., The Primitive Thylakoid-Less Cyanobacterium Gloeobacter Is a Common Rock-Dwelling Organism. Plos One 8 (2013).
39. M. S. Guzman et al., Phototrophic extracellular electron uptake is linked to carbon dioxide fixation in the bacterium Rhodopseudomonas palustris. Nature Communications 10 (2019).
40. X. Liu et al., Syntrophic interspecies electron transfer drives carbon fixation and growth by Rhodopseudomonas palustris under dark, anoxic conditions. Sci Adv 7 (2021).
41. F. W. Larimer et al., Complete genome sequence of the metabolically versatile photosynthetic bacterium Rhodopseudomonas palustris. Nature Biotechnology 22, 55-61 (2004).
42. S. Ganapathy et al., Retinal-Based Proton Pumping in the Near Infrared. Journal of the American Chemical Society 139, 2338-2344 (2017).
43. R. E. Blankenship et al., Comparing Photosynthetic and Photovoltaic Efficiencies and Recognizing the Potential for Improvement. Science 332, 805-809 (2011).
44. Y. Yang et al., Enhancing Bidirectional Electron Transfer of Shewanella oneidensis by a Synthetic Flavin Pathway. Acs Synthetic Biology 4, 815-823 (2015).
45. H. M. Lee, B. Y. Jeon, M. K. Oh, Microbial production of ethanol from acetate by engineered Ralstonia eutropha. Biotechnol Bioproc E 21, 402-407 (2016).
46. R. Simon, U. Priefer, A. Puhler, A Broad Host Range Mobilization System for Invivo Genetic-Engineering-Transposon Mutagenesis in Gram-Negative Bacteria. Bio-Technol 1, 784-791 (1983).
47. S. Green MR, J, Molecular Cloning: A Laboratory Manual, 4th edition (Cold Spring Harbor Laboratory Press, Cold Spring Harbor, NY, 2012).

### Tables:

TABLE 1
Strains and plasmids used in this study

| | Comments |
|---|---|
| Strains | |
| E. coli JM109 | Commercially obtained (Promega, UK) |
| Ralstonia eutropha H16 | Wild-type strain |
| (ATCC 17699) | Wild-type strain with phaCAB operon encoding genes |
| Ralstonia eutropha H16 | required for conversion of acetyl-CoA to |
| Δpha (RHM5) | polyhydroxybutyrate (PHB) deleted (gift from Min-Kyu |
| | Oh, Korea University, S. Korea). |
| Plasmids | |
| pLO11 (Tc^{r}, RK2 ori, Mob⁺) | Expression vector for use in R. eutropha with P_{BAD} |
| | promoter and downstream cloning sites (gift from Oliver |
| | Lenz, Technische Universität Berlin, Germany). |
| pLO11-CrtYI | pLO11 containing crtY and crtI from Erwinia herbicola |
| pLO11-CRT | pLO11 containing constitutive crtEXYIB operon from |
| | Erwinia uredovora |
| pLO11-Dxr | pLO11 containing dxr from R. eutropha |
| pLO11-DxrCRT | pLO11 containing dxr from R. eutropha downstream of |
| | P_{BAD} promoter and upstream of crtEXYIB operon from |
| | E. uredovora |
| pLO11-GR | pLO11 containing the gene for GR rhodopsin from |
| | Gloeobacter violaceus PCC7421 |
| pLO11-blh | pLO11 containing blh from the uncultured marine |
| | bacterium 66A03 (GenBank: DQ065755.1) codon |
| | optimised for R. eutropha |
| pLO11-Dxr-blh | pLO11 containing dxr and blh genes separated by a |
| | ribosome binding site |
| pLO11-blhDxrCRT | pLO11 containing dxr and blh genes separated by a |
| | ribosome binding site downstream of P_{BAD} promoter and |
| | upstream of crtEXYIB operon from E. uredovora |
| pLO11-blhDxrCRT-GR | pLO11-blhDxrCRT containing the GR gene downstream |
| | with its own P_{BAD} promoter |

### Supporting Information

### Materials and Methods

### Plasmid Construction

The detailed information about primers used in this study is shown in Supplementary Table 2.

The pLO11 (Tc^{r}, RK2 ori, Mob⁺) vector (1) containing the arabinose inducible P_{BAD} promoter specifically designed for expression in R. eutropha, was used for the expression of heterologous genes (gift from Oliver Lenz, Technische Universität Berlin, Germany).

The crtY and crtI genes were isolated by PCR as a single fragment from the pRER1B plasmid that contains the crtY and crtI genes from Erwinia herbicola (Pantoea agglomerans) (2) using the primers CrtYI_F and CrtYI_R and cloned into the NcoI-HindIII sites of pLO11 to create pLO11-CrtYI.

The Erwinia uredovora (Pantoea ananatis) crtEXYIB operon responsible for conversion of GGPP to β-carotene in E. coli, including the 879 bp upstream region required for constitutive β-carotene expression, was isolated by PCR as a single fragment from the pORANGE (3) plasmid (gift from Kwang-Hwan Jung, Sogang University, S.Korea) using the primers CRT_F and CRT_R and cloned into the SacI-Bg/II sites of pLO11 to create pLO11-CRT.

The dxr gene from R. eutropha (GenBank: CP039287.1) was synthesised (Integrated DNA Technologies, USA) and isolated by PCR using the primers SacDxr_F and SacDxr_R and cloned into the SacI site of the pLO11-CRT plasmid to create plasmid pLO11-DxrCRT. It was also isolated by PCR using the primers NcoDxr_F and HindDxr R and cloned into the Ncol-HindIII sites of pLO11 to create pLO11-Dxr.

The gene encoding the Gloeobacter violaceus PCC7421 rhodopsin, GR, (gift from Kwang Hwan-Lung, Sogang University, S.Korea; Genbank: BA000045) was isolated by PCR using the primer pair GR_F and GR_R and cloned into the Ncol and Bg/II sites of pLO11.

The β-carotene 15, 15′-dioxygenase gene (blh) from the uncultured marine bacterium 66A03 (GenBank: DQ065755.1) previously shown to produce retinal in E. coli (4) was codon optimised for R. eutropha and synthesised (Integrated DNA Technologies, USA) flanked by NcoI-HindIII sites and cloned into pLO11 to create pLO11-blh. The blh gene with an upstream ribosome binding site was created by PCR from pLO11-blh using primers Rbsblh_F and LOterm_R and inserted into the HindIII site of pLO11-Dxr to create pLO11-Dxr-blh. The dxr-blh operon was created by PCR from pLO11-Dxr-blh using primers Dxr_F and Blh_R and inserted into the HindIII site of pLO11-CRT to create pLO11-blhDxrCRT. The GR gene with its own P_{BAD} promoter was created by PCR from pLO11-GR using primers pBAD_F and LOterm_R and inserted into the EcoRI site of pLO11-blhDxrCRT to create pLO11-blhDxrCRT-GR.

### Carotenoid Extraction and Analysis

Carotenoids were solvent extracted from 50 ml cell culture pellets with 1 ml of 7:2 acetone: methanol (v/v), clarified by centrifugation and an absorption spectrum taken using an Agilent Cary 60 UV-Vis spectrophotometer. For more detailed analysis the solvent extract was dried down under nitrogen, re-dissolved in methanol and carotenoids separated by reversed-phase HPLC on an Agilent 1100 HPLC system using a Supelco Discovery HS C18 column as previously described (5). Elution of β-carotene and retinal were monitored at 450 nm and 380 nm respectively. An all trans-β-carotene standard (Sigma-Aldrich) of known concentration was used for the quantification of integrated peak areas. A pure all trans-retinal standard (Sigma-Aldrich) was used to assign the retinal peak.

### Membrane Purification

A 20 ml LB overnight culture with appropriate antibiotic selection of R. eutropha was used to inoculate 500 ml LB with selection in a 2.5 L conical flask. This was grown at 150 rpm and 30° C. to OD₆₀₀=0.5-07, induced with 0.1% (w/v) L-arabinose and 5 μg ml⁻¹ all trans-retinal and grown overnight at 30° C. The culture was pelleted and resuspended in approximately 10 ml of buffer A (25 ml K₂HPO₄/KH₂PO₄ pH 7.4) and lysed by two cycles of French pressing at a pressure of 18 000 psi. Membrane fractions were isolated on a 10-50% continuous sucrose gradient (the sucrose was made up in buffer A and 1.5 ml broken cells loaded per gradient) spun at 30,000 rpm for 2 hours at 4° C. A 1 ml membrane fraction was removed and an absorption spectrum taken using an Agilent Cary 60 UV-Vis spectrophotometer.

PHB Accumulation from Formate in the Light and Dark

The engineered R. eutropha precultured in TSB with 100 μg/mL kanamycin was centrifuged and washed three times with a nitrogen-limited minimal medium (3.57 g/L Na₂HPO₄, 1.5 g/L KH₂PO₄, 0.1 g/L (NH₄)₂SO₄, 0.08 g/L MgSO₄·7H₂O, 0.2 g/L NaHCO₃, 1 mg/L CaSO₄·2H₂O, 0.56 mg/L NiSO₄·7H₂O, 0.4 mg/L ferric citrate, and pH 7.0) to remove TSB medium. Subsequently, the cell pellet was resuspended with the nitrogen-limited minimal medium, and OD was adjusted to 1 before transferred into flasks with a total volume of 250 mL. Formate (80 mM) was added into the baffled flasks with a working volume of 200 mL for micro-aerobic PHB synthesis. The flasks were illuminated with a white LED light (˜50 μmol/s/m²) and dark wrapped in foil. Unless otherwise stated, all cultures were grown at 30° C. and 150 rpm.

### Results

Identification of Genes for Beta-Carotene Synthesis in R. eutropha H16

Analysis of the KEGG pathway for R. eutropha H16 (Cupriavidus nectar H16; https://www.genome.jp/kegg-bin/show pathway?reh00906) shows that it contains homologues of crtE and crtB (Genbank accession numbers CAJ92601.1 and CAJ93812.1 respectively) but not crtI and crtY. To see whether these homologous crtE and crtB genes were functional, and their products could be combined with exogenous CrtY and CrtI enzymes to make-carotene, the crtY and crtI genes were isolated as one fragment by PCR from the crt operon of Erwinia herbicola, where they are situated adjacent to each other in the crt operon, and inserted into the pLO11 (Tc^{r}, RK2 ori, Mob⁺) vector containing the arabinose inducible P_{BAD} promoter specifically designed for expression in R. eutropha (1). This plasmid pLO11-CrtYI (Table 1) was transformed into wild-type R. eutropha H16 by conjugation, the subsequent strain grown in the presence and absence of arabinose, the cell pellets subjected to solvent extraction and then analysed by HPLC. No β-carotene could be detected by HPLC in either the negative control consisting of R. eutropha H16 transformed with the empty pLO11 vector or the strain containing pLO11-CrtYI in either the absence or presence of arabinose (results not shown). This suggests that the native crtE and crtB genes in R. eutropha H16 are not functional in the β-carotene biosynthetic pathway.

The pORANGE plasmid, which gives constitutive β-carotene expression in E. coli JM109 (3), was used in attempts to synthesise this carotenoid in R. eutropha H16. DNA sequencing revealed that this plasmid encodes the carotenoid biosynthesis gene cluster (crtE, crtX, crtY, crtI, crtB; the crtX gene encodes the zeaxanthin glucosyltransferase enzyme which is not involved in β-carotene synthesis) from Pantoea ananatis (Erwinia uredovora) and an 879 bp region upstream of crtE that contains the putative 240 bp promoter region (Genbank: D90087) required for constitutive expression of this operon in E. coli JM109. The entire crtEXYIB operon, including the crtE promoter region (crt operon), was isolated by PCR from pORANGE and cloned into the pLO11 expression plasmid to create pLO11-CRT (designated pCRT; plasmid map shown in FIG. 5A).

Overexpression of the Dxr Gene in R. eutropha Enhanced β-Carotene Production

Isopentenyl diphosphate (IPP) is the common, five-carbon building block in the biosynthesis of all carotenoids with the second reaction in its synthesis being the reduction of 1-deoxy-D-xylulose 5-phosphate (DXP) to 2-C-methyl-D-erythritol-4-phosphate, catalysed by DXP reductoisomerase and encoded by dxr (FIG. 2A). Overexpression of the Synechocystis dxr gene in tobacco and the maize dxr gene in Zea mays has been shown to increase carotenoid content in tobacco and maize plants (6, 7). In order to try and increase the levels of β-carotene in R. eutropha H16 an attempt was made to overexpress the native dxr gene that encodes DXP reductoisomerase (EC: 1.1.1.267), the homologue of E. coli dxr, in conjunction with the crt operon. The dxr gene was synthesised and cloned into the pLO11-CRT plasmid to create plasmid pLO11-Dxr-CRT (designated pDxrCRT; plasmid map shown in FIG. 5B) in which expression was induced by arabinose. When this construct was transformed into E. coli JM109 the cells turned yellow without arabinose induction (FIG. 6A), whilst transformed R. eutropha H16 cells only appeared slightly yellow in the presence of arabinose (FIG. 6B). Subsequent solvent extraction of R. eutropha H16 pDxrCRT cells and HPLC analysis showed that β-carotene was produced after induction (FIG. 6C, solid line), whilst no β-carotene was detectable in the absence of arabinose (FIG. 6C, broken line). A negative control strain of R. eutropha H16, containing only the dxr gene cloned by PCR into the pLO11 expression plasmid produced no β-carotene in the absence or presence of arabinose (results not shown).

Redirection of Carbon Flux Promoted β-Carotene Production in R. eutropha

The pDxrCRT construct was transformed into the RHM5 AphaCAB strain (henceforth referred to as H16Δpha) as it has been shown that deleting the phaCAB operon encoding the three metabolic pathway genes from acetyl-CoA to polyhydroxybutyrate (PHB) increases production in R. eutropha of ethanol from acetate (8). The resulting strain looked demonstrably more yellow after induction with arabinose (FIG. 6D) and β-carotene could easily be extracted into solvent and detected using an absorbance scan (FIG. 6E) where the effect of induction on β-carotene production was clearly observable; subtraction of the uninduced scan from the induced gave a difference spectrum identical to that of β-carotene (FIG. 8F); HPLC analysis confirmed the presence in the solvent extract of trans- and cis-β-carotene observed as peaks eluting at about 16.9 minutes and 17.1 minutes respectively (FIG. 6G).

Three weighed cell pellets from 50-ml cultures of R. eutropha H16 pCRT and H16 pDxrCRT were each solvent extracted, the extract analysed by HPLC to determine the mean integrated β-carotene peak areas (as determined by Agilent ChemStation HPLC software) and compared against the peak areas obtained from several dilutions of a 1 mg/ml β-carotene standard dissolved in methanol. Yield values for R. eutropha H16 pCRT and pDxrCRT of approximately 0.6=0.2 ug and 0.9+0.1 μg β-carotene per g wet weight of pellet respectively were obtained showing that more β-carotene was produced in the presence of dxr. A further improvement in yield was obtained with R. eutropha H16Δpha pDxrCRT where a value of approximately 2+0.5 μg β-carotene per g wet weight of pellet was obtained (FIG. 6H).

Validation of GR Biosynthesis in E. coli and R. eutropha H16 Using Single Cell Raman Micro-Spectroscopy

In order to determine the presence in vivo of the GR-retinal complex in cells expressing the GR gene, samples of uninduced and arabinose induced H16Δpha GR, JM109 blhDxrCRT-GR and H16Δpha blhDxrCRT-GR strains were examined at a single-cell level by Raman spectroscopy. Induction of H16Δpha GR included the addition of exogenous retinal whereas induction of JM109 blhDxrCRT-GR, H16Δpha blhDxrCRT-GR and H16 blhDxrCRT-GR did not as these strains make their own endogenous retinal. Single-cell Raman spectra (SCRS) were obtained (n>150 in each induced sample and n>100 in each uninduced sample) and the averaged spectra are shown in FIG. 8A. A prominent band at 1530 cm⁻¹ was present in all induced samples of H16Δpha GR (labelled H16-GR), JM109 blhDxrCRT-GR (labelled JM109) and H16Δpha blhDxrCRT-GR (labelled H16); this band was also present in the H16 blhDxrCRT-GR strain (where the pha operon has not been deleted), appearing upon arabinose induction (FIG. 8B). This characteristic Raman band was attributed to ethylenic stretching (vc=c) vibrations in retinal-protein complexes (9), which depending on the carbon chain length and structure, can shift between 1505-1530 cm⁻¹ (10). To date, Raman spectroscopy has only been carried out on purified GR protein either as crystals (11) or reconstituted into liposomes (12) where the ethylenic stretching (vc=c) band was typically seen between 1524-1535 cm⁻¹ depending on the pH conditions.

The percentage of cells expressing GR-retinal in a population was calculated by counting the number of SCRS that contained the 1530 cm⁻¹ GR band (Table 3). The induced JM109 blhDxrCRT-GR sample had 41% of its population present with GR-retinal whilst 9% of the uninduced population also had GR-retinal present which is presumably a reflection of some ‘leakiness’ in the P_{BAD} promoter. Induced samples of H16Δpha blhDxrCRT-GR and H16Δpha GR showed 8% and 89% respectively of the populations expressing GR-retinal; no Raman band at 1530 cm⁻¹ was observed in the uninduced samples. The percentage of cells expressing GR-retinal (Table 3) was highest in the H16Δpha GR strain, to which exogenous retinal had been added, and is presumably a reflection of the reduced amount of endogenous retinal produced in E. coli JM109 and R. eutropha H16Δpha by the pLO11 blhDxrCRT-GR construct, although the higher percentage of cells in the former indicates better expression of the construct in E. coli. As the amount of endogenous retinal is the limiting factor in the production of GR-retinal there is certainly more scope for improving its production, for example, through codon optimisation of all the genes, the use of different promoters with differing induction conditions and molecular evolution/screening techniques.

### Neutral Red-Mediated CO₂ Reduction.

Neutral red (NR) is a common electron shuttling molecule. Cyclic voltammetry (CV) verified reversible redox reactions of NR at the carbon cloth electrode. The cyclic voltammogram shows typical oxidative and reductive peaks, indicating that carbon cloth is a suitable material in terms of NR-mediated electron transfer. As the midpoint potential was around −0.52 V versus Ag/AgCl electrode, for subsequent growth experiments, a potential of −0.6 V versus Ag/AgCl electrode was applied at the working electrode (cathode) to reduce the mediator (NR). Moreover, we also confirmed the electron transfer from NR to R.eutropha. As shown in FIG. 8, in the presence of cells, neutral red was obviously oxidized with the supernatant colour changing from pink to yellow. After centrifugation, the cell pellet was visibly pink compared to the control without neutral red, which indicated that NR entered cells and was oxidized within the R. eutropha cells. Therefore, we confirmed the circuit from electrode to cells. In absence of NR, there was no cell growth. In comparison, obvious increases in cells were observed in the presence of NR. Notably, the cells with GR expression showed a significant growth in the light compared to other groups, similar to growth experiments using formate.

### SUPPORTING INFORMATION REFERENCES

1. A. Schwarze, M. J. Kopczak, M. Rogner, O. Lenz, Requirements for construction of a functional hybrid complex of photosystem I and [NiFe]-hydrogenase. Appl Environ Microbiol 76, 2641-2651 (2010).
2. C. N. Hunter et al., Introduction of New Carotenoids into the Bacterial Photosynthetic Apparatus by Combining the Carotenoid Biosynthetic Pathways of Erwinia-Herbicola and Rhodobacter-Sphaeroides. J Bacteriol 176, 3692-3697 (1994).
3. S. Y. Kim, S. A. Waschuk, L. S. Brown, K. H. Jung, Screening and characterization of proteorhodopsin color-tuning mutations in Escherichia coli with endogenous retinal synthesis. Biochim Biophys Acta 1777, 504-513 (2008).
4. Y. S. Kim, C. S. Park, D. K. Oh, Retinal production from beta-carotene by beta-carotene 15,15′-dioxygenase from an unculturable marine bacterium. Biotechnol Lett 32, 957-961 (2010).
5. L. A. Malone et al., Cryo-EM structure of the spinach cytochrome b (6) f complex at 3.6 angstrom resolution. Nature 575, 535-+ (2019).
6. T. Hasunuma et al., Overexpression of 1-Deoxy-D-xylulose-5-phosphate reductoisomerase gene in chloroplast contributes to increment of isoprenoid production. J Biosci Bioeng 105, 518-526 (2008).
7. J. Hans, B. Hause, D. Strack, M. H. Walter, Cloning, characterization, and immunolocalization of a mycorrhiza-inducible 1-deoxy-d-xylulose 5-phosphate reductoisomerase in arbuscule-containing cells of maize. Plant Physiol 134, 614-624 (2004).
8. Lee, H.M., Jeon, B. Y. & Oh, M.K. Microbial production of ethanol from acetate by engineered Ralstonia eutropha. Biotechnol Bioproc E 21, 402-407 (2016).
9. Y. Z. Song et al., Proteorhodopsin Overproduction Enhances the Long-Term Viability of Escherichia coli. Appl Environ Microb 86, (2020).
10. J. Jehlicka et al., Potential and limits of Raman spectroscopy for carotenoid detection in microorganisms: implications for astrobiology. Philos TR Soc A 372, (2014).
11. T. Morizumi et al., X-ray Crystallographic Structure and Oligomerization of Gloeobacter Rhodopsin. Sci Rep-Uk 9, (2019).
12. M. R. M. Miranda et al., The Photocycle and Proton Translocation Pathway in a Cyanobacterial Ion-Pumping Rhodopsin. Biophys J 96, 1471-1481 (2009).

All references discussed herein are incorporated by reference.

SUPPLEMENTARY TABLE 2
primers used in this study

| Primer name | Sequence |
|---|---|
| CrtYI_F (SEQ ID NO: 19) | GATCCATGGGAAGGGATCTGATTTTAGTCGGC |
| CrtYI_R (SEQ ID NO: 20) | GATAAGCTTGCTCATTGCAGATCCTCAATCA |
| CRT_F (SEQ ID NO: 21) | GCTCTAGAGCTCGATCTCAAACAGGATTGGGC |
| CRT_R (SEQ ID NO: 22) | GCTCTAGATCTGACGCTCCGGGAAAGAC |
| SacDxr_F (SEQ ID NO: 23) | GCGGAGCTCATGCATCGCATTACCATCCTG |
| SacDxr_R (SEQ ID NO: 24) | GCGGAGCTCTCAGCGCTTTGCAGCC |
| NcoDxr_F (SEQ ID NO: 25) | GCACCATGGGCCATCGCATTACCATCCTGGG |
| HindDxr_R (SEQ ID NO: 26) | GCAAAGCTTCAGCGCTTTGCAGCC |
| pBAD_F (SEQ ID NO: 27) | GCAGATCTGAATTCAAGAAACCAATTGTCCA |
| | TATTGCATC |
| LOterm_R (SEQ ID NO: 28) | GCTGAATTCAGATCTGTGACGCAGTAGCGGT |
| | AAACG |
| GR_F (SEQ ID NO: 29) | CATGCCATGGGTTTGATGACCGTATTTTCTTC |
| GR_R (SEQ ID NO: 30) | GCAGATCTGCGGCCGCTAGGAGATAAGACTGC |
| | CTCCCG |
| Rbsblh_F (SEQ ID NO: 31) | GCGAAGCTTAAGGAGGAGACCCCATGG |
| Dxr_F (SEQ ID NO: 32) | GCGGAGCTCATGGGCCATCGCATTACCATC |
| Blh_R (SEQ ID NO: 33) | GCGGAGCTCTTATTAATGGTGATGGTGATGA |
| | TGGC |

TABLE 3
The percentage of cells with GR in the induced or uninduced populations.

| Cells with GR | | | | | | |
|---|---|---|---|---|---|---|
| (1520 cm⁻¹ | JM109 blhDxrCRT-GR | | H16Δpha blhDxrCRT-GR | | H16Δpha -GR | |
| band) | induced | uninduced | induced | uninduced | induced | uninduced |
| Percentage | 41% | 9% | 8% | 0% | 89% | 0% |
| (GR cells/ | (62/153) | (9/102) | (13/155) | (0/103) | (136/153) | (0/103) |
| total cells) | | | | | | |

TABLE 4
The percentage of cells with GR in Ralstonia eutropha
H16 blhDxrCRT-GR under microaerobic or aerobic conditions
before and after growth for 72 h without antibiotics.

| | Total cells | | |
|---|---|---|---|
| Conditions | measured | Cells with GR | Percentage |
| 0 h | 200 | 165 | 82.5% |
| Microaerobic 72 h light | 400 | 277 | 69.2% |
| Microaerobic 72 h dark | 400 | 148 | 36.8% |
| Aerobic 72 h light | 400 | 86 | 26.2% |
| Aerobic 72 h dark | 400 | 143 | 35.8% |

### Example 2—Engineering Artificial Photosynthesis in Bacteria for CO₂ Fixation with Gloeobacter Rhodopsin (GR) and an Additional Outer-Membrane Conduit Mtr

### SUMMARY

Here we constructed a rhodopsin-based photosynthetic electron transport chain in a chemoautotrophic bacterium Ralstonia eutropha H16 to achieve photoelectro-autotrophic growth. As designed, Gloeobacter rhodopsin (GR) and an outer-membrane conduit Mtr were heterogeneously expressed, which were integrated into R. eutropha's native electron transport chain (ETC). In a hybrid photoelectrochemical system, water was broken to release electrons from an electrode to the engineered cells via the Mtr pathway coupled with flavin. Then the light-activated GR with canthaxanthin as an antenna drove the ETC in reverse to generate reductants, by which carbonic anhydrase-overexpressing cells fixed CO₂ into biomass with a faradic efficiency of ˜45%. This system represents previously unidentified phototrophic metabolism based on Mtr-rhodopsin-ETC and may fundamentally change the current perception of rhodopsin-based photosynthesis.

### INTRODUCTION

We previously engineered the autotrophic bacterium R. eutropha H16 with a Gloeobacter rhodopsin (GR) and created a redox loop by integrating it with an extracellular electrode. The electrode-supplied electrons can be transferred into the R. eutropha for driving CO₂ fixation, mediated by an electron-shuttling molecule flavin and powered by rhodopsin. This system can effectively incorporate CO₂ but low electron transfer rate and efficiency could be improved. We hypothesised that establishing an efficient electron transfer interface on the cell membrane is a key approach to improving the electron movement from the electrode to the cells. Natural electroactive bacteria have an outer membrane-bound Mtr pathway allowing bidirectional electron transfer between extracellular substrates and the quinone pool. Inspired by the Mtr-mediated inward electron transfer, we artificially combined extracellular electron uptake with intracellular reverse electron transfer to create a synthetic photosynthetic electron transport chain for powering efficient CO₂ fixation.

In this study, R. eutropha was engineered to heterogeneously express the MtrCAB complex and GR protein to construct an electrochemically driven photosynthetic electron transport chain, which is integrated with a solar-driven electrochemical system to achieve artificial photosynthesis (FIG. 16). Similar to the natural photosynthesis, water is broken into oxygen, hydrogen ions and electrons in the anode. Unlike the common electrosynthesis, the electrons would be transferred from a cathode to cells mediated by outer membrane-bound Mtr that is connected to the inner membrane-bound electron transport chain, rather than via synthesis of intermediates such as H₂ and formate. Then the light-powered rhodopsin drives the electron transfer in reverse to regenerate NADH and NADPH for CO₂ fixation. The biomass of R. eutropha was chosen as the end product because it produces high levels of protein, as well as other important nutrients such as essential amino acids and vitamins, making it an attractive alternative to traditional animal feed sources such as soybeans or corn¹⁶. To enhance the efficiency of the system, flavin and canthaxanthin were introduced to combine with Mtr and GR, respectively. Additionally, a carbonic anhydrase (can) was overexpressed to improve the system which was reported important in concentrating CO₂¹⁷ and favourable to CO₂ fixation¹⁸. Overall, the photo-electrosynthesis established in this study could suggest a potential strategy for conversion of CO₂ to valuable chemicals, and the designed phototrophic metabolisms could help improve the understanding of underlying metabolic processes in nature.

### Results

### Engineering a Synthetic Mtr-Mediated Electron Transport Chain

A plasmid containing MtrCAB (pLO11a-MtrCAB) was created under the control of a P_{BAD} promoter. MtrCAB is a multi-heme protein complex linking the intracellular electron transport chain with extracellular substrates (FIG. 17a). Cytochrome c maturation is required for heme insertion¹⁹. Unlike E. coli which cannot express cytochrome c aerobically¹⁹, single-cell Raman analysis shows that R. eutropha cells can synthesize cytochromes under aerobic conditions, the same as that of electroactive S. oneidensis MR-1 cells (FIG. 21). Therefore, plasmid pLO11a-MtrCAB was transferred into R. eutropha to create R. eutropha-Mtr. After induction with arabinose, the cell pellet of induced R. eutropha-Mtr showed a red colour (inlets in FIG. 17b), compared with that of uninduced cells. The red colour was attributed to the presence of Mtr complexes on the cell membrane which is consistent with the results reported in E. coli¹⁹. Single-cell Raman analysis shows that Raman spectra of cells expressing Mtr display a significant increase in bands associated with the cytochromes (FIG. 17b and Supplementary FIG. 17b); conversely, a previous study reported that S. oneidensis MR-1's cytochromes levels decreased due to the deletion of Mtr genes²⁰. MtrC and MtrA are c-type cytochromes; thus, the elevated cytochromes suggest the synthesis of Mtr in R. eutropha-Mtr. Electrochemical tests were performed to further confirm the functions of Mtr in R. eutropha. Since R. eutropha has a well-known nitrate-reducing metabolism²¹, we hypothesised that in the presence of nitrate as an electron acceptor, the Mtr pathway in R. eutropha-Mtr could obtain cathodic electrons for denitrification via native nitrate reductases (FIG. 17a). The precultured R. eutropha-Mtr with induction and the uninduced strain were incubated in potentiostatic-controlled bioreactors under cathodic conditions (−500 mV_{Ag/AgCl}). After a period of acclimation, 20 mM nitrate was added to the cathodic chamber. We observed that the addition of nitrate accelerated R. eutropha-Mtr consuming current and a significant current drop was observed over ˜6 h, while little current change was found in the control group with the Mtr-uninduced strain (FIG. 17c). These observations indicate that electron flux from a cathode to nitrate reductases is dependent on the Mtr complex. The nitrate respiration pathway is part of the electron transport chain; thus, the expression of Mtr creates an electrical connection between cellular energy metabolism and the electrode. Notably, in the model electroactive bacterium S. oneidensis MR-1, MtrCAB is coupled with an inner-membrane protein CymA to involve electron transfer, but recent studies showed that CymA is not necessary for inward electron transport²². Several other inner-membrane enzymes such as NapC can function as CymA^{19,23}. Our results suggest that in R. eutropha-Mtr system, MtrCAB is sufficient to deliver electrons from a cathode to the nitrate-reducing pathway via its native inner-membrane proteins.

Combination of the Electron Transport Chain with a Rhodopsin-Based Photosystem

Reducing power such as NADH and NADPH is the key driving force to power CO₂ fixation. Although the Mtr-mediated electron transfer system can support electron flow inwardly, there is an energy hurdle over intracellular electron transfer. We expect that electrons flow from a cathode to Mtr (−300 mV_{SHE} to +100 mV_{SHE})²⁴, then to the quinones pool (−80 mV_{SHE} for menaquinone) mediated by inner-membrane proteins and onto NADH dehydrogenase for the generation of NADH (−320 mV_{SHE}). However, the electron transfer from quinol to NAD⁺ is thermodynamically unfavourable. Previous studies have proved that introducing a rhodopsin-based proton pump can drive the process by providing extra energy in the form of proton motive force^{9,10}. Therefore, in this study, we combined the Mtr pathway with a gene encoding Gloeobacter rhodopsin (GR) to form a phototrophic extracellular electron uptake pathway (FIG. 17d). The GR-based photosystem can harvest light energy around 530 nm8 and act as a proton pump. Previous in-vitro studies have demonstrated that holo-GR was able to generate a proton motive force under light and it had a two-fold faster turnover rate than other proteorhodopsins (PRs)²⁵. In this study, a plasmid containing MtrCAB and GR (pLO11a-MtrCAB-GR) was transferred to R. eutropha to create R. eutropha-GR-Mtr. Due to the expression of GR, the cell pellet of arabinose-induced cells showed a pink colour, compared with that of uninduced bacteria (FIG. 17g). The pink colour was attributed to the presence of GR-retinal complexes on the cell membrane. Single-cell Raman analysis shows that Raman spectra of cells expressing GR display a characteristic rhodopsin band at 1530 cm⁻¹ (FIG. 17e), consistent with the previous report. The GR complexes were detected in 87% of the cells in the induced (number of measured cells n=270) and none in the uninduced population (number of measured cells n=306) (FIG. 17f). After establishing the GR-based photosystem with the Mtr pathway, the R. eutropha-GR-Mtr strain was used to investigate the possibility that the extracellular electrons could drive the reducing power generation in R. eutropha under light, by incubating the strain in cathodic conditions with an electrode as the electron source. After 2-day incubation, reducing power was accumulated in light due to the energy generated by GR. The NADH and NADPH levels were significantly enhanced in the light compared to that in the dark (FIG. 17h). Interestingly, in the light, the NADPH/NADP⁺ ratio was higher than NADH/NAD⁺ (FIG. 17h), indicating that the strain could be more inclined to uptake extracellular electrons for driving NADPH-dependent reactions such as CO₂ fixation.

Light-Driven Metabolism with CO₂ as the Sole Carbon Source

It is reasonable to assume that the reducing power NADPH would energize the CO₂ fixation pathway of R. eutropha (i.e., Calvin cycle). Biosynthesis requires a catalyst, energy and building blocks (such as CO₂ and H₂O). In this study, R. eutropha-GR-Mtr was used as a catalyst to investigate its capability of utilising light energy to fix CO₂. Heavy water D₂O was used as a tracer to probe cellular metabolisms under different conditions. We hypothesised that the presence of both CO₂ and energy can drive H₂O (and D₂O) involved in the metabolisms including the generation of NAD (P) H and the synthesis of 3-phosphoglycerate (3PG), then the metabolites would be labelled with deuterium (FIG. 23a).

The precultured R. eutropha-GR-Mtr was inoculated into cathode chambers which are operated under four conditions: dark and no CO₂, dark and supplying CO₂, light and no CO₂, and light and supplying CO₂. After 2-day incubation, obvious C-D vibrations were only observed in the group with light and CO₂ in both averaged Raman spectra (FIG. 18a) and single-cell analysis (FIG. 18b; number of measured cells≈400), suggesting that the above-mentioned NADPH can be used to drive the CO₂ fixation pathway. In particular, the single band at 1003 cm⁻¹ is characteristic of the phenyl ring of phenylalanine (an aromatic amino acid), which becomes three bands at 987, 975, and 961 cm⁻¹ due to the utilisation of D₂O (FIG. 23b). These isotopic shifts are in good agreement with the phenylalanine deuteration reported in previous work²⁶. In addition, the band at 1050 cm⁻¹ which could be associated with bicarbonate was found in cells supplied with CO₂ under darkness. A continuous supply of CO₂ can acidify the medium and cells whilst lacking sufficient reduction energy to consume CO₂ resulting in the accumulation of bicarbonate intracellularly. In summary, these observations confirmed that R. eutropha-GR-Mtr has been converted to a photoelectro-autotrophic bacterium.

### Hybrid Photosynthesis for Biomass Growth on CO₂

To achieve photosynthesis with light energy as the sole energy source, we employed a solar panel to drive the electrochemical platform. Instead of using a potentiostat to control the cathode potential, we designed a voltage regulator to adjust the applied potential generated by the solar cells (FIG. 24 a and b). The voltage regulator can also stabilise the cathode potential to avoid cathode potential fluctuations that could harm cells.

R. eutropha's biomass was chosen as the end product of photosynthesis, as it was qualified by European Union to be used as a safe source of single-cell protein for animal feed¹⁶. phaCAB operon was knocked out in R. eutropha to create R. eutropha A Apha (RHM5) mutant for maximising carbon flux towards biomass (FIG. 19a). Flavin mononucleotide (FMN) was added as an electron mediator to enhance the electron transfer rate which can react with MtrC²⁷. Single-cell Raman analysis showed a typical band of FMN at ˜1340 cm⁻¹ in cells expressing Mtr (FIG. 19b and FIG. 25), indicating that exogenous FMN could bind to MtrC as the cofactor involved in electron transport. The RHM5-GR-Mtr strains were inoculated in bioelectrochemical reactors with an applied voltage of 1.8 V under light or dark over 5 days, whilst CO₂ was continuously pumped in as the sole carbon source. The results showed that cells only grew on CO₂ in the presence of light (FIG. 26). The OD₆₀₀ of the induced RHM5-GR-Mtr increased to 0.237 over 5-day illumination (FIG. 19f). The control group did not show significant biomass production, ruling out the possibility of H₂-mediated electron transfer to support growth. These results suggest that we engineered R. eutropha capable of growing on CO₂ powered by the photosynthetic electron transport chain in light. Our previous work added exogenous riboflavin as an electron shuttle to mediate electron transfer between GR-expressing R. eutropha and the electrode. In this study, we compared the effects of MtrCAB on cell doubling time and system faradic efficiency. In the presence of the Mtr pathway, cells showed almost halved doubling time, and the faradic efficiency increased to 35.4% from 23.9% compared to the non-Mtr flavin-mediated system (FIGS. 19g and h). These results suggest that the expression of the outer membrane-bound MtrCAB establishes a specific electron transfer path to connect extracellular electrons with central carbon metabolism. In comparison, the flavin-mediated system seems unspecific and less efficient without the involvement of the Mtr complex.

To further increase the efficiency of the photosystem, canthaxanthin was added as an antenna of GR to improve the capture of light energy (FIG. 19a). The GR-canthaxanthin complex was reported to possess a 5-fold proton pumping capacity compared to sole GR²⁸. Single-cell Raman analysis showed typical bands of canthaxanthin at 1005, 1155 and 1517 cm⁻¹ in cells with the GR-retinal complex (FIG. 19c and FIG. 25b). The addition of canthaxanthin changed the colour of the cell suspension of GR-expressing cells from pink to red (inlet in FIG. 19c), which is consistent with the previous study²⁸. In the presence of canthaxanthin, the doubling time of the photo-electrotrophic growth reduced to 70.1 h (FIG. 19g) and the efficiency increased to 42.9% (FIG. 19h), suggesting that GR with canthaxanthin can effectively enhance the photosynthetic electron transfer.

In addition to energy generation module, the CO₂ fixation module was also improved by overexpressing a native carbonic anhydrase can which preferably catalyses the conversion of bicarbonate to CO₂²⁹. Due to a part of CO₂ combined with water to form bicarbonate and then transported inwards cells, abundant can is expected to increase the CO₂ utilisation rate. When ¹²C-formate and ¹³C-bicarbonate were used as the substrates to culture the can-expressing RHM5, single-cell Raman analysis showed isotopic shifts of the phenylalanine band at 1003 cm⁻¹ to 987, 975, and 961 cm⁻¹, indicating an incorporation of bicarbonate into biomass (FIG. 19d and FIG. 27). We further calculated the degree of the ¹³C incorporation and found a higher ¹³C content in can-expressing RHM5 compared to controls (FIG. 19e). In the photoelectrochemical system, the overexpression of can further reduced the generation time to a minimum of 67.3 h (FIG. 19f) which is nearly comparable to that of a native anoxygenic phototrophic bacterium Rhodopseudomonas palustris under electrochemical conditions (40 h˜50 h) 30. The maximum faradic efficiency of the engineered strains with additional canthaxanthin can reach 45.0% (FIG. 19h), indicating that the modular engineering can effectively enhance the hybrid photosynthesis.

### DISCUSSION

This is the first study constructing a hybrid photosynthetic electron transport chain in R. eutropha to convert a chemoautotroph to a photoautotroph. To mimic natural chlorophyll-based photosynthesis, we introduced a solar-driven electrochemical system to function as photosystem II for water splitting and installed rhodopsin as photosystem I providing extra energy to direct electron transfer (FIG. 20). The outer membrane of the cells is like a boundary separating in vitro water splitting from in vivo NADPH regeneration and CO₂ fixation. Unlike common H₂-mediated artificial photosynthesis³¹, we engineered a transmembrane conduit Mtr complex as an interface integrating the intracellular electron transport chain with an extracellular cathode. In theory, extracellular electron transfer could achieve a comparable efficiency to H₂-mediated system³². To ensure Mtr-mediated mechanism is the major process leading to electron transfer, we applied a relatively low potential at 1.8 V to avoid hydrogen evolution (FIG. 20). 1.8 V has proved hard to support sufficient cell growth in other electrochemical systems under normal conditions^{31,33}. The form of photosynthetic electron transport chain designed in this study is fundamentally similar to natural photosynthesis in which electrons are transferred via a series of redox enzymes accompanied by proton movement, without the synthesis of new intermediates such as H₂³¹ and formate³⁴ (Table 6). Such Yin (electrons) and Yang (protons) interactions drive the photochemical reactions of photosynthesis.

The artificial photosynthesis described here is designable and optimisable compared to natural photosynthesis which has been evolving for billions of years to be structurally complex. In this study, the addition of flavin and canthaxanthin enhanced the electron transfer rate and the proton pumping rate, respectively. Materials engineering like electrode modification can also provide effective approaches to increasing energy efficiency³⁵. It is even possible to achieve maximum utilisation of light energy by extending rhodopsin's light absorption which has already proven feasible for the GR photosystem₃₆. Such modification is incredibly challenging for chlorophyll-based photosystems.

### Materials and Methods

### Bacterial Strains, Culture Conditions and Plasmid Construction.

All bacterial strains, plasmids, and primers used in this study are shown in Table 5 and FIG. 28. E. coli strains were grown in Lysogeny Broth (LB) at 37° C. under aeration by shaking at 200 rpm. R. eutropha and S. oneidensis were grown in LB broth at 30° C. under aeration by shaking at 150 rpm. If required, antibiotics (Sigma-Aldrich) were added as follows: 10 μg ml⁻¹ gentamicin and 10 μg ml⁻¹ tetracycline for R. eutropha; 12.5 μg ml⁻¹ tetracycline for E. coli. Before preculture of bio-electrochemical experiments, R. eutropha strains were grown in TSB (Tryptic Soy Broth) and minimal medium with fructose as the carbon source. The minimal medium was prepared as and filter sterilized, composed of 6.74 g/L Na₂HPO4·7H₂O, 1.5 g/L KH₂PO₄, 1.0 g/L (NH₄)₂SO₄, 1 mg/L CaSO₄·2H₂O, 80 mg/L MgSO₄·7H₂O, 0.56 mg/L NiSO₄·7H₂O, 0.4 mg/L ferric citrate, 200 mg/L NaHCO₃, 1 mL/L concentrated metals solution (1.5 g/l FeCl₂·4H₂O, 0.19 g/l CoCl₂·6H₂O, 0.1 g/l MnCl₂·4H₂O, 0.07 g/l ZnCl₂, 0.062 g/l H₃BO₃, 0.036 g/l Na₂MoO₄·2H₂O, 0.025 g/l Na₂WO₄·2H₂O and 0.017 g/l CuCl₂·2H₂O), 10 ml/L concentrated vitamin solution (2 mg/L D-biotin, 2 mg/L folic acid, 10 mg/L pyridoxine HCl, 5 mg/L thiamine HCl, 5 mg/L nicotinic acid, 5 mg/mL D-pantothenic acid, hexacalcium salt, 0.1 mg/L cobalamin, 5 mg/L p-aminobenzoic acid, 5 mg/L α-lipoic acid and 5 mg/L FMN) and 10 mL/L concentrated amino acids solution (2 g/L L-glutamic acid, 2 g/L L-arginine, and 2 g/L D, L-serine), and pH was adjusted to 7. For anabolic test of the overexpression carbonic anhydrase in R. eutropha, the R. eutropha mutant with pLO11-can were pre-grown overnight in the minimal medium with and without 0.2% (w/v) arabinose, respectively, using 20 mM fructose as carbon sources. Then the precultured strains were washed three times with the minimal medium and inoculated in the minimal medium with 20 mM formate and 10 mM ¹³C-bicarbonate (initial OD=0.5). After 24-hours culture, cells were collected for single-cell Raman analysis.

The plasmid pLO11a-Mtr containing the MtrCAB biosynthesis gene cluster was introduced to synthesize the MtrCAB complex in R. eutropha H16-Mtr. The plasmid pLOlla-GR containing the Gloeobacter rhodopsin gene (GR) was introduced to make GR in R. eutropha H16-GR10. pLO11a-GR-Mtr containing GR and MtrCAB was introduced to R. eutropha Δpha to make RHM5-GR-Mtr. pLO11a-GR-Mtr-can containing GR, MtrCAB, and can was introduced to make R. eutropha Δpha to make RHM5-GR-Mtr-can. Induction of strains transformed with the pLOlla expression vector containing the arabinose-inducible P_{BAD} promoter was carried out by growth to log phase and the addition of 0.2% (w/v) L-arabinose (Sigma-Aldrich) and overnight growth at 30° C. for R. eutropha. Induction of GR expression was accompanied by the addition of exogenous trans-retinal (Sigma-Aldrich) to a final concentration of 5 μg ml⁻¹. Exogenous trans-canthaxanthin (Sigma-Aldrich) was added as an antenna for GR with a final concentration of 5 μg ml⁻¹. Flavin mononucleotide (FMN) was also added to be anchored on the Mtr with a concentration of 10 μmol/L.

Cloning procedures were performed according to standard protocols. Polymerase chain reaction (PCR) was carried out using Q5 DNA polymerase (NEB, UK) and synthesised primers (Sigma-Aldrich) according to the manufacturer's instructions. The construction of plasmid was performed in E. coli DH5α using HiFi assembly (NEB, USA). After extraction and purification, the plasmid was transferred into R. eutropha strains by conjugation.

### Single-Cell Raman Spectra (SCRS) Measurements and Analysis

Single bacterial cells, their metabolic profiles and pure standard chemicals were characterized by Raman microspectroscopy. Prior to the measurements, bacterial cells were washed three times with distilled water to remove traces of culture medium and extracellular metabolites. The intactness of the cells was observed under a microscope after washing. Cells were diluted to a degree that individual bacteria could be observed with a 1.5-μl suspension dropped onto an aluminium-coated slide and air dried. Raman spectroscopic acquisition was performed using a LabRAM HR Evolution confocal Raman microscope using a 100×/0.75 air objective (HORIBA, UK). Single-cell Raman spectra (SCRS) were obtained using a 532-nm neodymium-yttrium aluminium garnet laser with a 300 grooves mm⁻¹ diffraction grating and were acquired in the range of 100-3200 cm⁻¹. The laser power was set at ˜80 mW which was attenuated by neutral density (ND) filters before focusing onto the samples. Spectra were recorded with LABSPEC 6 software (HORIBA, UK). All raw spectra were pre-processed by cosmic ray correction, polyline baseline fitting and subtraction and vector normalization of the entire spectral region. Linear discriminant analysis (LDA) was used for dimension reduction of SCRS to aid visualization at the single-cell level. All analysis and plotting were done under an R 4.0.0 environment.

### Analysis and Quantification of Intracellular Biomolecules

For characterizing cells under induced or uninduced conditions with arabinose, triplicates were performed in each condition and Raman spectra were acquired using a 25% power filter and 3 to 5-second acquisition time with high signal-to-noise ratios. Raman bands at 748 (pyrrole breathing mode), 1128 (v(CN) stretching vibrations), 1312 (δ (CH) deformations) and 1584 cm⁻¹ (v (CC) skeletal stretches) were attributed to cytochromes. Quantification of cytochrome c was done by integrating band areas at these four band positions. Intracellular phenylalanine was identified and quantified by the phenylalanine band centered at 1003 cm⁻¹. Raman spectra of pure riboflavin 5′-monophosphate sodium salt hydrate (Sigma-Aldrich, UK) in powder as well as in water solution was measured as a standard to identify a band at 1340 cm⁻¹ as an indicator for quantification of flavin, which is assigned to the middle ring vibration of the tricyclic isoalloxazine structure of the flavin. Gloeobacter rhodopsin (GR) complexes were characterized by Raman microscopy using a 1% power filter and 1-second acquisition time. The low power and short acquisition time were used to prevent photobleaching of the GR chromophores. SCRS of strains with the pLO11a expression vector, either with or without the addition of L-arabinose were measured in triplicates. GR complexes were identified by a band at ˜1530 cm⁻¹ above the background noise in the SCRS. Quantification of biomolecules was done by integrating the area of the corresponding Raman bands.

The degree of ¹³C incorporation into biomass was determined by calculating the isotopic shifts of phenylalanine from 1003 cm⁻¹ to 987, 975, and 961 cm⁻¹, as in previous research²⁶. Depending on different ¹³C substitutions on the phenylalanine ring, a total of four possible Raman positions of the ¹³C/¹²C mixture existed for possible isotopomers. Due to the symmetric structure, substitutions at only three carbon sites on the phenyl ring can affect the vibrational modes hence the wavenumbers: the bands at 1003 cm⁻¹ correspond to structures where all three carbon are 12C; the bands at 987 cm⁻¹ occur when any of the three carbon sites are substituted with 13C; the 975-cm⁻¹ bands occur when any two of the sites are 13C; and the bands at 961 cm⁻¹ only appear when all three carbon sites are ¹³C and hence occur only with high assimilation from ¹³C-bicarbonate into biomass. The degree of ¹³C incorporation was calculated by the ratio of total ¹³C to total ¹³C and 12C as follows, in which A represents the area under the curve centered at a defined wavenumber:

$$Total {}^{13} C = \frac{{A}_{961 c {m}^{- 1}} + \frac{2}{3} {A}_{975 c {m}^{- 1}} + \frac{1}{3} {A}_{987 c {m}^{- 1}}}{{A}_{975 c {m}^{- 1}} + {A}_{987 c {m}^{- 1}} + {A}_{1003 c {m}^{- 1}}}$$

### Determination of NADH and NADPH Levels

For measurements of intracellular reductant levels including NADH and NADPH, cells were harvested from the working chamber and pre-treated according to the manufacturer's instructions. NADH/NAD⁺ and NADPH/NADP⁺ ratios were determined using a NAD/NADH Assay Kit (Abcam, USA) and an NADP/NADPH Assay kit (Sigma-Aldrich, UK) and were measured using a colourimetric assay with a microplate reader (BioTek Corporation, UK) detecting the wavelength at 450 nm.

### Fluorescence Microscopy to Visualize Biofilm on the Electrode

The Raman microscope chassis was modified with an epifluorescence system in consultation with the manufacturer (HORIBA, UK). An LED lamp and a FITC filter block were used to visualize the fluorescence of the engineered cells stained by SYTO™ 9 (Thermo Fisher Scientific, UK) with a 20×/0.4. The final images were processed with background denoising using ImageJ.

### Microbial Photoelectrochemical System

A dual-chamber bioreactor (70 mL of total volume r) separated by a Nafion membrane (only allowing proton transfer) was used as a bio-photoelectrochemical system. The microbial photoelectrochemical experiments were performed in a three-electrode configuration on a multichannel potentiostat (PalmSens, Netherlands). For the anode chamber, a stainless-steel mesh was used as the counter electrode. For the cathode chamber, the working electrode was made of 2.5×4.0 cm² carbon cloth and an Ag/AgCl reference electrode (3M KCl, RE-5B, BASi, USA) was installed for measuring the potentials. The anodic electrolyte was made of 50 mM KH₂PO₄ and 50 K₂HPO₄ mM, and the cathodic electrolyte was the same as the above-mentioned minimal medium. The working chamber was continuously agitated by a magnetic stirrer at 30° C. For the photoelectrochemical experiments, 5 m of white LED light strips were set adjacent to the working chamber and illumination intensity was monitored by a photometer.

### Electrochemically Driven Nitrate Reduction

To test electron transfer from the electrode to cells, arabinose-induced and uninduced R. eutropha-Mtr were inoculated to the cathode of the photoelectrochemical system. R. eutropha-Mtr strains were first activated 24 h in TSB in the presence of 10 μg ml⁻¹ tetracycline. Then 200 μL of bacterial cultures were inoculated into 10 mL of the fresh minimal medium on 10 mM fructose and 60 mM formate with 10 μg ml⁻¹ tetracycline. For the Mtr induction group, 0.2% (w/v) L-arabinose was added to induce the gene expression. After overnight preculture in a shaker with 150 rpm at 30° C., the cells were harvested by centrifugation at 3000 g for 3 min and washed three times with the minimal medium to remove organics. The cell pellets were resuspended in the minimal medium and the OD₆₀₀ was adjusted to ˜0.5 before transfer to the working chamber which was continuously bubbled with N₂ gas to maintain an anaerobic environment. As advised by a previous study²², the cells were subjected to anodic conditions at +200 mV_{Ag/AgC1} for two days to exhaust any potential internal electron storage, such as PHB, and increase cell adhesion to the electrode. After a period of assimilation, the electrode potential was then switched to the cathodic condition at −500 m V_{Ag/AgCl}. 20 mM of nitrate was added to monitor the current change by a potentiostat (PalmSens, Netherlands).

### Electrochemically Driven Photosynthetic Electron Transport Chain for Reductant Generation

R. eutropha-GR-Mtr was used to test whether the electrode-supplied electrons can be used to regenerate reducing power NADH and NADPH. The uninduced and induced strains were precultured as above and inoculated into the microbial photoelectrochemical system with an initial OD of ˜0.5. During the experiments, the working chambers were poised at −500 mV_{Ag/AgC1} and bubbled with N². The reactors were illuminated with a white LED light (˜150 μmol/s/m²) for the light tests or covered with aluminium foil as darkness control. After 2 days of R. eutropha-GR-Mtr incubation in the light and dark, cells were harvested from the working chamber for the measurement of NADH and NADPH.

### The Use of Heavy Water (D20) to Probe Phototrophic Metabolisms

The R. eutropha-GR-Mtr was precultured as described above. The 40% D₂O (v/v) minimal medium was prepared with 99% D20 (v/v) (Sigma, UK) to resuspend the cells pellet. The initial OD was adjusted to ˜0.2 before being inoculated into the working chamber which was sparged with N₂ to remove oxygen and then changed to CO₂. After 2 days of incubation, single-cell Raman analysis was used to detect the C-D vibration in the cells.

A Hybrid System for Light-Driven CO₂ Fixation into Biomass.

For light-driven experiments, a two-electrode configuration was performed with the carbon cloth as the cathode and platinum as the anode. A commercial polycrystalline solar cell (1.5 W, 140 mm×180 mm, RS, UK) was combined with a homemade voltage regulator to power the water splitting to generate electrons. To maximize the carbon conversion to biomass, the RHM5 strain, an R. eutropha mutant without PHB biosynthetic pathways was used as a biocatalyst. The precultured RHM5-GR-Mtr strains were treated as above before being inoculated into the cathode chamber. Then the cells were pre-grown in the cathode chamber under formatotrophic conditions with 60 mM formate to form a biofilm on the electrode. After 2 days of incubation when the biocathode was established, the spent medium was replaced with fresh medium, and the biomass (OD₆₀₀) of the planktonic cells was adjusted to ˜0.1. The growth experiments were conducted by continuously sparging with an 80:20 mixture of N₂: CO₂ gas under light and dark conditions, with and without induction of GR-Mtr or GR-Mtr-can, respectively. FMN was introduced as an electron mediator to boost the electron transfer. 12.5 μmol/L of flavin was added at an interval of 24 h to reduce their light destruction.

### Calculations

The electron transfer efficiency (n) of the biomass formation could be calculated by the equation,

$$η = \frac{n z F}{Charge passed ( C )}$$

Where n is the amount of biomass (using the formula CH_{1.77}O_{0.49}N_{0.24}³¹ and 1 OD₆₀₀ corresponding to 0.448 g/L dry biomass) product (mol), z is the number of transferred electrons (z=4 for CO₂ conversion to biomass), F is the Faraday constant (96,485 C/mol).

The doubling time (g) of the strains could be calculated by the equation,

$$g = \frac{ln 2}{ln ( O {D}_{d a y 5} / O {D}_{d a y 1} ) / 4}$$

Where OD_{day5} and OD_{day1} represent the OD values of the planktonic cells on Day 5 and Day 1, respectively.

### REFERENCE

1 Kolber, Z. S. et al. Contribution of Aerobic Photoheterotrophic Bacteria to the Carbon Cycle in the Ocean. Science 292, 2492-2495 (2001). https://doi.org:10.1126/science.1059707
2 Worden, A. Z. et al. Rethinking the marine carbon cycle: Factoring in the multifarious lifestyles of microbes. Science 347, 1257594 (2015). https://doi.org:doi:10.1126/science.1257594
3 Field, C. B., Behrenfeld, M. J., Randerson, J. T. & Falkowski, P. Primary Production of the Biosphere: Integrating Terrestrial and Oceanic Components. Science 281, 237-240 (1998). https://doi.org:doi:10.1126/science.281.5374.237
4 Gómez-Consarnau, L. et al. Microbial rhodopsins are major contributors to the 4 solar energy captured in the sea. Science Advances 5, eaaw8855 (2019). https://doi.org:doi:10.1126/sciadv.aaw8855
5 Béjà, O. et al. Bacterial Rhodopsin: Evidence for a New Type of Phototrophy in the Sea. Science 289, 1902-1906 (2000). https://doi.org:doi:10.1126/science.289.5486.1902
6 Palovaara, J. et al. Stimulation of growth by proteorhodopsin phototrophy involves regulation of central metabolic pathways in marine planktonic bacteria. Proceedings of the National Academy of Sciences 111, E3650-E3658 (2014). https://doi.org:10.1073/pnas.1402617111
7 Jing, X. et al. Revealing CO₂-Fixing SAR11 Bacteria in the Ocean by Raman-Based Single-Cell Metabolic Profiling and Genomics. BioDesign Research 2022 (2022).https://doi.org:doi:10.34133/2022/9782712
8 Tu, W. & Huang, W. E. Rhodopsin driven microbial CO₂ fixation using synthetic biology design. Environmental Microbiology 25, 126-130 (2023). https://doi.org:https://doi.org/10.1111/1462-2920.16243
9 Tefft, N. M. & TerAvest, M. A. Reversing an Extracellular Electron Transfer Pathway for Electrode-Driven Acetoin Reduction. ACS Synthetic Biology 8, 1590-1600 (2019). https://doi.org:10.1021/acssynbio.8b00498
10 Davison, P. A. et al. Engineering a Rhodopsin-Based Photo-Electrosynthetic System in Bacteria for CO₂ Fixation. ACS Synthetic Biology 11, 3805-3816 (2022). https://doi.org:10.1021/acssynbio.2c00397
11 Walter, J. M., Greenfield, D., Bustamante, C. & Liphardt, J. Light-powering Escherichia coli with proteorhodopsin. Proceedings of the National Academy of Sciences 104, 2408-2412 (2007). https://doi.org:doi:10.1073/pnas.0611035104
12 Sano, M. et al. Conversion of Mevalonate to Isoprenol Using Light Energy in Escherichia coli without Consuming Sugars for ATP Supply. ACS Synthetic Biology 11, 3966-3972 (2022). https://doi.org:10.1021/acssynbio.2c00313
13 Toya, Y. et al. Optogenetic reprogramming of carbon metabolism using light-powering microbial proton pump systems. Metabolic Engineering 72, 227-236 (2022). https://doi.org:https://doi.org/10.1016/j.ymben.2022.03.012
14 Hu, G. et al. Light-driven CO2 sequestration in Escherichia coli to achieve theoretical yield of chemicals. Nature Catalysis 4, 395-406 (2021). https://doi.org:10.1038/s41929-021-00606-0
15 Rowe, A. R. et al. Tracking Electron Uptake from a Cathode into Shewanella Cells: Implications for Energy Acquisition from Solid-Substrate Electron Donors. mBio 9, e02203-02217 (2018). https://doi.org:doi:10.1128/mBio.02203-17
16 Sillman, J. et al. Bacterial protein for food and feed generated via renewable energy and direct air capture of CO₂: Can it reduce land and water use? Global Food Security 22, 25-32 (2019). https://doi.org:https://doi.org/10.1016/j.ofs.2019.09.007
17 Lu, J., Brigham, C. J., Li, S. & Sinskey, A. J. in Biotechnology for Biofuel Production and Optimization (eds Carrie A. Eckert & Cong T. Trinh) 325-351 (Elsevier, 2016).
18 Thorbecke, R. et al. The gene dosage effect of carbonic anhydrase on the biosynthesis of poly(3-hydroxybutyrate) under autotrophic and mixotrophic culture conditions. Polymer Journal 53, 209-213 (2021). https://doi.org:10.1038/s41428-020-00409-3
19 Jensen, H. M. et al. Engineering of a synthetic electron conduit in living cells. Proceedings of the National Academy of Sciences 107, 19213-19218 (2010). https://doi.org:doi:10.1073/pnas 1009645107
20 Ng, C. K. et al. Elevated intracellular cyclic-di-GMP level in Shewanella oneidensis increases expression of c-type cytochromes. Microbial Biotechnology 13, 1904-1916 (2020). https://doi.org:https://doi.org/10.1111/1751-7915.13636
21 Cramm, R. Genomic view of energy metabolism in Ralstonia eutropha H16. J Mol Microbiol Biotechnol 16, 38-52 (2009). https://doi.org:10.1159/000142893
22 Baruch, M., Tejedor-Sanz, S., Su, L. & Ajo-Franklin, C. M. Electronic control of redox reactions inside Escherichia coli using a genetic module. PLOS ONE 16, e0258380 (2021). https://doi.org:10.1371/journal pone 0258380
23 Feng, J. et al. Direct electron uptake from a cathode using the inward Mtr pathway in Escherichia coli. Bioelectrochemistry 134, 107498 (2020). https://doi.org:https://doi.org/10.1016/j.bioelechem 2020.107498
24 Ross, D. E., Flynn, J. M., Baron, D. B., Gralnick, J. A. & Bond, D. R. Towards Electrosynthesis in Shewanella: Energetics of Reversing the Mtr Pathway for Reductive Metabolism. PLOS ONE 6, e16649 (2011). https://doi.org:10.1371/journal.pone.0016649
25 Chen, Q., Arents, J., Ganapathy, S., de Grip, W. J. & Hellingwerf, K. J. Functional Expression of Gloeobacter Rhodopsin in Synechocystis sp. PCC6803. Photochemistry and Photobiology 93, 772-781 (2017). https://doi.org:https://doi.org/10.1111/php. 12745
26 Xu, J. et al. Raman Deuterium Isotope Probing Reveals Microbial Metabolism at the Single-Cell Level. Analytical Chemistry 89, 13305-13312 (2017). https://doi.org:10.1021/acs analchem 7b03461
27 Okamoto, A. et al. Cell-secreted Flavins Bound to Membrane Cytochromes Dictate Electron Transfer Reactions to Surfaces with Diverse Charge and pH. Scientific Reports 4, 5628 (2014). https://doi.org:10.1038/srep05628
28 Chuon, K. et al. The role of carotenoids in proton-pumping rhodopsin as a primitive solar energy conversion system. Journal of Photochemistry and Photobiology B: Biology 221, 112241 (2021). https://doi.org:https://doi.org/10.1016/j.jphotobiol.2021.112241
29 Gai, C. S., Lu, J., Brigham, C. J., Bernardi, A. C. & Sinskey, A. J. Insights into bacterial CO₂ metabolism revealed by the characterization of four carbonic anhydrases in Ralstonia eutropha H16. AMB Express 4, 2 (2014). https://doi.org:10.1186/2191-0855-4-2
30 Guzman, M. S. et al. Phototrophic extracellular electron uptake is linked to carbon dioxide fixation in the bacterium Rhodopseudomonas palustris. Nature Communications 10, 1355 (2019). https://doi.org:10.1038/s41467-019-09377-6
31 Liu, C., Colón, B. C., Ziesack, M., Silver, P. A. & Nocera, D. G. Water splitting-biosynthetic system with CO₂ reduction efficiencies exceeding photosynthesis. Science 352, 1210-1213 (2016). https://doi.org:doi:10.1126/science.aaf5039
32 Rowe, A. R. et al. Identification of a pathway for electron uptake in Shewanella oneidensis. Communications Biology 4, 957 (2021). https://doi.org:10.1038/s42003-021-02454-x
33 Torella, J. P. et al. Efficient solar-to-fuels production from a hybrid microbial& #x2013;water-splitting catalyst system. Proceedings of the National Academy of Sciences 112, 2337-2342 (2015). https://doi.org:doi:10.1073/pnas 1424872112
34 Li, H. et al. Integrated electromicrobial conversion of CO₂ to higher alcohols. Science 335, 1596 (2012). https://doi.org:10.1126/science. 1217643
35 Catania, C., Karbelkar, A. A. & Furst, A. L. Engineering the interface between electroactive bacteria and electrodes. Joule 5, 743-747 (2021). https://doi.org:https://doi.org/10.1016/j.joule.2021.02.001
36 Engqvist, M. K. M. et al. Directed Evolution of Gloeobacter violaceus Rhodopsin Spectral Properties. Journal of Molecular Biology 427, 205-220 (2015). https://doi.org:https://doi.org/10.1016/i.imb.2014.06.015
37 Hassanzadeh, B. et al. Microbial rhodopsins are increasingly favoured over chlorophyll in High Nutrient Low Chlorophyll waters. Environmental Microbiology Reports 13, 401-406 (2021). https://doi.org:https://doi.org/10.1111/1758-2229.12948
38 Finkel, O. M., Béjà, O. & Belkin, S. Global abundance of microbial rhodopsins. The ISME Journal 7, 448-451 (2013). https://doi.org:10.1038/ismej.2012.112
39 Pushkarev, A. et al. A distinct abundant group of microbial rhodopsins discovered using functional metagenomics. Nature 558, 595-599 (2018). https://doi.org:10.1038/s41586-018-0225-9
40 Chazan, A. et al. Phototrophy by antenna-containing rhodopsin pumps in aquatic environments. Nature 615, 535-540 (2023). https://doi.org:10.1038/s41586-023-05774-6
41 Baker, I. R., Conley, B. E., Gralnick, J. A. & Girguis, P. R. Evidence for Horizontal and Vertical Transmission of Mtr-Mediated Extracellular Electron Transfer among the <i>Bacteria</i>. mBio 13, e02904-02921 (2022). https://doi.org:doi:10.1128/mbio.02904-21
42 Liu, X. et al. Syntrophic interspecies electron transfer drives carbon fixation and growth by <i>Rhodopseudomonas palustris</i>under dark, anoxic conditions. Science Advances 7, eabh1852 (2021). https://doi.org:doi:10.1126/sciadv.abh1852
43 Kato, S. Microbial extracellular electron transfer and its relevance to iron corrosion. Microbial Biotechnology 9, 141-148 (2016). https://doi.org:https://doi.org/10.1111/1751-7915.12340

### Supplementary Information for Example 2

TABLE 5
Strains and plasmids used in this study

| | Comments |
|---|---|
| Strains | |
| E. coli DH5α | Commercially obtained (NEW England, UK) |
| E. coli S17 | Commercially obtained (NEW England, UK) |
| E. coli MG1655 | Commercially obtained (NEW England, UK) |
| Shewanella oneidensis MR- | Wild-type strain |
| 1 | |
| Ralstonia eutropha H16 | Wild-type strain |
| (ATCC 17699) | |
| Ralstonia eutropha H16 | Wild-type strain with phaCAB operon encoding genes |
| Δpha (RHM5) | required for conversion of acetyl-CoA to |
| | polyhydroxybutyrate (PHB) deleted (gift from Min- |
| | Kyu Oh, Korea University, S. Korea). |
| Plasmids | |
| pLO11a (Tc^{r}, RK2 ori, | Expression vector for use in R. eutropha with P_{BAD} |
| Mob⁺) | promoter and downstream cloning sites (gift from |
| | Oliver Lenz, Technische Universität Berlin, Germany). |
| pLO11a-GR | pLO11a containing the gene for GR rhodopsin from |
| | Gloeobacter violaceus PCC7421 |
| pLO11a-Mtr | pLO11a containing MtrCAB gene cluster from |
| | Shewanella oneidensis MR-1 |
| pLO11a-GR-Mtr | pLO11a containing GR gene rhodopsin from |
| | Gloeobacter violaceus PCC7421 and MtrCAB gene |
| | cluster from Shewanella oneidensis MR-1 |
| pLO11a-can | pLO11a containing can gene from Ralstonia eutropha |
| pLO11a-GR-Mtr-can | pLO11a containing GR gene rhodopsin from |
| | Gloeobacter violaceus PCC7421, MtrCAB gene cluster |
| | from Shewanella oneidensis MR-1 and can gene from |
| | Ralstonia eutropha |

TABLE 6
Comparison to other microbial electrochemical systems for
conversion of CO₂ fixation into R. eutropha biomass.

| | Electron | Applied | | | |
|---|---|---|---|---|---|
| Strains | uptake | potential | | η_{elec-to-biomass} | Reference |
| R. eutropha H16 | H₂ | 2.7 | V | 13% | (1) |
| R. eutropha H16 | H₂ | 2.0 | V | 54% | (2) |
| R. eutropha H16 | H₂ | ~5 | V | 4.8% | (3) |
| R. eutropha H16 | Formate | ~4 | V | 1.8% | (4) |
| R. eutropha H16 | / | 1.8 | V | No growth | (1, 2) |
| R. eutropha H16 | Flavin | 1.8 | V | 20% | (5) |
| with GR | | | | | |
| R. eutropha H16 | Extracelluar | 1.8 | V | 45% | This study |
| Δpha (RHM5) with | electron | | | | |
| GRCX | transfer | | | | |
| | and flavin | | | | |

(1) Torella J P, Gagliardi C J, Chen J S, et al. Efficient solar-to-fuels production from a hybrid microbial-water-splitting catalyst system. Proceedings of the National Academy of Sciences, 2015, 112(8): 2337-2342.
(2) Liu C, Colón B C, Ziesack M, et al. Water splitting-biosynthetic system with CO2 reduction efficiencies exceeding photosynthesis. Science, 2016, 352(6290): 1210-1213.
(3) Schlegel H G, Lafferty R. Growth of ‘Knallgas’ bacteria (Hydrogenomonas) using direct electrolysis of the culture medium. Nature, 1965, 205(4968): 308-309.
(4) Li H, Opgenorth P H, Wernick D G, et al. Integrated electromicrobial conversion of CO2 to higher alcohols. Science, 2012, 335(6076): 1596-1596.
(5) Davison P A, Tu W, Xu J, et al. Engineering a rhodopsin-based photo-electrosynthetic system in bacteria for CO2 fixation. ACS Synthetic Biology, 2022, 11(11): 3805-3816.

1. An electromicrobial system for photoautotrophic CO₂ fixation, comprising:
a recombinantly-engineered bacterial cell configured for photoautotrophic CO₂ fixation, wherein the recombinantly-engineered bacterial cell comprises a membrane-bound rhodopsin molecule and a CO₂ fixation pathway; and
an electron source.

2. The electromicrobial system according to claim 1, wherein the recombinantly-engineered bacterial cell comprises a recombinantly-engineered R. eutropha cell.

3. The electromicrobial system according to claim 1, wherein the membrane-bound rhodopsin molecule comprises a proteorhodopsin (PR) or a Gloeobacter rhodopsin (GR).

4. (canceled)

5. The electromicrobial system according to claim 1, wherein the recombinantly-engineered bacterial cell is configured for retinal biosynthesis.

6. The electromicrobial system according to claim 1, wherein the recombinantly-engineered bacterial cell comprises retinal.

7. The electromicrobial system according to claim 5, wherein:
the recombinantly-engineered bacterial cell is configured for β-carotene biosynthesis; and
the recombinantly-engineered bacterial cell expresses β-carotene 15, 15′-dioxygenase.

8. The electromicrobial system according to claim 1, wherein the recombinantly-engineered bacterial cell comprises dxs, dxr, ispH, ispA, crtE, crtB, crtI, crtY, and/or blh.

9. The electromicrobial system according to claim 1, wherein:
the recombinantly-engineered bacterial cell expresses 1-deoxy-D-xylulose-5-phosphate synthase, 1-deoxy-D-xylulose 5-phosphate reductoisomerase, 4-hydroxy-3-methylbut-2-enyl diphosphate reductase, farnesyl diphosphate synthase, geranylgeranyl diphosphate synthase, phytoene synthase, phytoene desaturase, lycopene cyclase, and/or β-carotene 15, 15′-dioxygenase;
the recombinantly-engineered bacterial cell expresses 1-deoxy-D-xylulose-5-phosphate synthase, 1-deoxy-D-xylulose 5-phosphate reductoisomerase, 4-hydroxy-3-methylbut-2-enyl diphosphate reductase, farnesyl diphosphate synthase, geranylgeranyl diphosphate synthase, phytoene synthase, phytoene desaturase, and/or lycopene cyclase, wherein the recombinantly-engineered bacterial cell further expresses β-carotene 15, 15′-dioxygenase;
the recombinantly-engineered bacterial cell expresses 1-deoxy-D-xylulose-5-phosphate synthase, 1-deoxy-D-xylulose 5-phosphate reductoisomerase, 4-hydroxy-3-methylbut-2-enyl diphosphate reductase, farnesyl diphosphate synthase, geranylgeranyl diphosphate synthase, phytoene synthase, phytoene desaturase, lycopene cyclase, and/or β-carotene 15, 15′-dioxygenase;
the recombinantly-engineered bacterial cell comprises: i) crtI, crtY, crtE and crtB; and ii) a gene encoding-carotene 15, 15′-dioxygenase; or
the recombinantly-engineered bacterial cell: i) is a R. eutropha cell; ii) comprises crtI and crtY; and iii) comprises functional, endogenous crtE and crtB genes.

10. The electromicrobial system according to claim 1, wherein the recombinantly-engineered bacterial cell comprises an Erwinia uredovora (Pantoea ananatis) crtEXYIB operon.

11. The electromicrobial system according to claim 1, wherein:
the recombinantly-engineered bacterial cell is configured for expression of β-carotene 15, 15′-dioxygenase;
the recombinantly-engineered bacterial cell is further configured to overexpress an endogenous dxr gene; and/or
the recombinantly-engineered bacterial cell comprises a recombinant dxr gene.

12. The electromicrobial system according to claim 1, wherein the electron source comprises an electrode linked to a source of electricity and/or an electron donor molecule.

13. (canceled)

14. The electromicrobial system according to claim 1, wherein an electron is mediated via an electron mediator.

15. The electromicrobial system according to claim 1, wherein the electron source comprises an electrode disposed in a dual chamber electrode system.

16. The electromicrobial system according to claim 1, further comprising riboflavin.

17. The electromicrobial system according to claim 1, wherein the recombinantly-engineered bacterial cell is configured for the production of an organic product.

18. A bacterial cell configured for photoautotrophic CO₂ fixation, wherein the bacterial cell comprises a rhodopsin and the components for a CO₂ fixation pathway.

19. The bacterial cell according to claim 18, wherein the bacterial cell comprises an R. eutropha cell.

20. The bacterial cell according to claim 18, wherein the rhodopsin comprises a proteorhodopsin (PR) or a Gloeobacter rhodopsin (GR).

21. (canceled)

22. The bacterial cell according to claim 18, wherein the bacterial cell is configured for retinal biosynthesis.

23. (canceled)

24. A method of CO₂ fixation and/or biosynthesizing an organic product, the method comprising:
providing the electromicrobial system according to claim 1; and
culturing the recombinantly-engineered bacterial cell in the presence of light and CO₂.

25-29. (canceled)
